(19) United States
(12) Patent Application Publication
(10) Pub. No.: US 2017/0320942 A1
(43) Pub. Date: Nov. 9, 2017

(54) BISPECIFIC MOLECULE BINDING TLR9 AND CD32 AND COMPRISING A T CELL EPITOPE FOR TREATMENT OF ALLERGIES

(71) Applicant: S-TARGET THERAPEUTICS GMBH, Vienna (AT)

(72) Inventors: Geert Mudde, Breitenfurt bei Wien (AT); Gottfried Himmler, Groß-Enzersdorf (AT)

(21) Appl. No.: 15/607,661

(22) Filed: May 29, 2017

Related U.S. Application Data
(63) Continuation-in-part of application No. 15/248,258, filed on Aug. 26, 2016.
Application No. 15/248,258 is a continuation of application No. 12/281,504, filed on Sep. 3, 2008, now abandoned.

(30) Foreign Application Priority Data
Mar. 3, 2006 (EP) 06110672.0

(51) Int. Cl. C07K 16/28; C07K 14/435; A61K 39/00

(52) U.S. Cl. CPC C07K 16/283; C07K 16/2896; C07K 14/43531; C07K 2319/00; C07K 2317/66; C07K 2317/53; A61K 39/00; C07K 2317/34; C07K 2317/31; A61K 2039/57; C07K 2319/40; C07K 2319/74; C07K 2317/526

(57) ABSTRACT

A molecule or molecule complex capable of binding to TLR9 and to CD32 comprising at least one epitope of at least one antigen, and its use a medicament for the treatment of allergies.

### CROSS-REFERENCE TO RELATED APPLICATIONS

This application claims the benefit of priority under 35 U.S.C. §119 from European Patent Application No. 06110672.0, filed Mar. 3, 2006, and claims the benefit of priority under 35 U.S.C. §120 from the following U.S. patent applications: U.S. patent application Ser. No. 15/248,258, filed Aug. 26, 2016; U.S. patent application Ser. No. 13/791,824, filed Mar. 8, 2013; and U.S. patent application Ser. No. 12/281,504, filed September 3, which is the U.S. national stage of International Patent Application No. PCT/EP2007/001722, filed Feb. 28, 2007. The contents of the foregoing patent applications are hereby incorporated by reference in their entirety.

### SEQUENCE LISTING

The entire content of a Sequence Listing titled “Sequence_Listing.txt,” created on May 18, 2017 and having a size of 68 kilobytes, which has been submitted in electronic form in connection with the present application, is hereby incorporated by reference herein in its entirety.

### FIELD OF THE INVENTION

The present invention relates to molecules with binding specificity to both, Toll-like Receptor 9 (TLR9) and CD32 containing one or more T cell antigen epitopes. The invention further relates to the production of these molecules and their use for the preparation of medicaments for the treatment of allergies.

### BACKGROUND

Allergy is considered to be a hypersensitive reaction to proteins in the environment (air/water/food). Allergens are antigens to which atopic patients respond with IgE antibody responses subsequently leading to allergic reactions. Antigens in the complexes or fusion proteins can be environmental allergens (e.g. house dust mite, birch pollen, grass pollen, cat antigens, cockroach antigens), or food allergens (e.g. cow milk, peanut, shrimp, soya), or a combination of both. IgE molecules are important because of their role in effector cell (mast cell, basophiles and eosinophils) activation. More recently, it has been accepted that IgE also plays an important role in the induction phase of allergic diseases, by up-regulating the antigen capture potential of B cells and dendritic cells (DC), both through low affinity (CD23) and high affinity receptors (FcεRI) [1]. The negative functions of IgE antibodies can be counteracted by allergen specific IgG antibodies. e.g. because they direct the immune response away from B cells to monocytes and DC [2]. In addition, they compete with IgE molecules for allergen binding sites. Allergies therefore can be treated, cured and prevented by the induction of allergen specific IgG molecules.

IgG molecules have a serum half-life of approximately 3 weeks as compared to roughly 3 days for IgE molecules. IgE molecules are induced by the interaction between (naive) B cells and Th2 cells which provide the IL-4 and IL-13 together with CD40L expression necessary to induce a class switch to IgE in memory B cells and plasma cells [3]. In contrast, TM cells, which produce IFN-γ and IL-2, induce a class switch to IgG. Therefore, induction of Th1, rather than Th2 helper T cell responses against allergens, is beneficial for the prevention, treatment and cure of allergic diseases.

To date several forms of active vaccination using allergens are used. The most common is the so called “Immunotherapy”, which depends on frequent immunizations with relatively high concentrations of allergens. This technique is only moderately effective in a minority of allergic diseases such as Bee venom allergy and in some cases of Rhinitis and Conjunctivitis, and recently some reports have shown effectiveness in asthma and atopic dermatitis. More recently rush immunotherapy, where increasing amounts of allergen are injected in a rather short time frame, has been proposed with slightly better results [4; 5]. Usually the subcutaneous route is used for administration of the allergens, but recently this route has been compared to oral application or even local application, the results are generally positive but not always consistent. A different technique for immunotherapy is the one described by Saint-Remy (EP 0 178 085 and 0 287 361), which makes use of autologous IgG antibodies which are in vitro complexed to the relevant allergens. This technique allows far smaller amounts of allergen to be applied with fewer side effects.

The mechanism behind these therapies is unclear. In the classical therapy there seems to be a beneficial effect if the therapy induces an increase in specific IgG antibodies, although not every significant increase of specific IgG is correlated with successful immunotherapy. A possible argument why this is the case is the relatively low affinity of IgG antibodies for CD32 on B cells, monocytes and mast cells. The Saint-Remy approach selects the specific IgG antibodies from the patient, which are subsequently mixed with relevant allergens in vitro. This way they assure that the allergen cannot react freely with cells or other antibody isotypes on cells such as IgE on mast cells. In addition they claim that anti-idiotypic antibodies are raised against the specific IgG molecules, which in the future will prevent allergy.

In WO 97/07218 Allergen-anti-CD32 Fusion Proteins are described. In this publication the problems with isolating specific IgG molecules and the low affinity of these IgG antibodies for CD32 are circumvented and the risk factors of classical immunotherapy, which uses complete “IgE binding” allergens, are reduced. However, the claimed induction of Th1 memory responses due to solely directing the anti-CD32 containing vaccine to dendritic cells cannot be substantiated.

Even in view of the intensive research for therapeutic approaches to treat allergic diseases, there is still a great demand for providing medicaments for successful treatment of allergies.

The object of the invention is therefore to provide novel molecules with improved properties for the treatment of allergic diseases.

According to the invention this object is achieved by the subject matter of the claims.

### BRIEF SUMMARY OF THE INVENTION

CD32 is strongly expressed on monocytes/dendritic cells and B cells and thus the molecule of the present invention is designed to direct the immune response to these important immunological cells, with the intention to prevent allergen presentation by the B cells, while promoting allergen presentation by especially dendritic cells (DCs), the latter leads to induction of Th1 responses against the allergens in the molecule or molecule complex that can be formulated as vaccine. More recent knowledge shows that two types of dendritic cells (DC) exist: myeloid (mDC) and plasmacytoid dendritic cells (pDC) [6], which has led to the new concept of DC1 and DC2 cells [7]. In this concept DC1 cells promote the induction of Th1 cell development after antigen specific stimulation and DC2 cells support the development of Th2 cells. Monocyte derived DC (or mDC) are generally considered to be of DC1 type, whereas pDC are considered to be DC2 type [6]. Both types of DC express CD32a and will induce an allergen specific T cell response; however it is not guaranteed that the outcome will be of Th1 type. In fact, in allergic donors Th2 responses are more likely [8]. Importantly, the pDC express the TLR9 receptor, which binds CpG-ODNs (oligodeoxynucleotides [ODNs] containing unmethylated CpG motifs). Activation of this receptor in the pDC leads to a very strong production of IFN-α and IL-12 [9], which promotes Th1 induction and thus transforms the potential DC2 into DC1 cells.

Therefore, the molecule of the invention can combine the activation of the TLR9 receptor in pDC with the specific stimulation and induction of allergen specific Th1 cells and comprises therefore a significant improvement of earlier concepts.

The invention comprises a molecule or a molecule complex having binding specificity for toll-like receptor 9 and CD32, wherein the molecule or molecule complex includes at least one epitope, preferably at least one T cell epitope, of at least one antigen. The molecule or molecule complex of the invention will also bypass the effector function of mast cells, which carry IgE, for the native allergen of which T cell epitopes have been selected to be part of the fusion protein.

Preferably the molecule or molecule complex according to the invention can have one or more of the following unique characteristics:
Activation and induction of allergen specific Th1 cells, without activation of allergen-specific B-cells.
Activation and induction of allergen specific Th1 cells, without activation of mast cells or any other effector cell, which, by means of allergen specific IgE or IgG, may become activated by the natural allergens of which the selected T cell epitopes are represented in the molecule or molecule complex of the invention.

The CD32-binding part of the molecule or molecule complex of the invention selects the relevant cells, which should internalize the complete molecule or molecule complex.

After internalization of the fusion protein according to the present invention by antigen presenting cells the molecule of the invention is degraded and various peptides, in eluding the incorporated T cell epitope(s) are presented on the MHC class Il molecules of the antigen presenting cells, therefore stimulating allergen specific T cells.

The incorporated TLR9-binding structure(s) in the molecule or molecule complex of the invention are necessary for the induction of an allergen specific Th1 memory pool, by binding to the cytoplasmatic [10; 11] TLR9 receptor. Activation of the TLR9 receptor leads to a strong induction of IFN-α and IL12 production [9].

According to the present invention, a molecule is a single entity made up of atoms and/or other molecules by covalent bonds. The molecule can be made up of one single class of substances or a combination thereof. Classes of substances are e.g. polypeptides, carbohydrates, lipids, nucleic acids etc.

A molecule complex is an aggregate of molecules specifically and strongly interacting with each other. A complex of various molecules may be formed by hydrophobic interactions (such as e.g. the binding of antibody variable regions in an Fv) or by strong binding of one molecule to another via ligand/receptor interactions such as antibody-antigen binding or avidin-biotin or by complex formation via chelating chemical groups and the like. The molecule complex is preferably produced through chemical conjugation, recombinant fusion and/or affinity binding.

An antigen can be a structure which can be recognized by an antibody, a B-cell-receptor or a T-cell-receptor when presented by MHC class I or Il molecules.

An epitope is the smallest structure to be specifically bound within by an antibody, a B-cell-receptor or a T-cell receptor when presented by MHC class I or Il molecules. Specificity is defined as preferred binding to a certain molecular structure (in antibody/antigen interactions also called epitope) within a certain context.

A domain is a discrete region found in a protein or polypeptide. A monomer domain forms a native three-dimensional structure in solution in the absence of flanking native amino acid sequences. Domains of the invention will specifically bind to CD32 and/or TLR9 and/or display or present epitopes. Domains may be used as single domains or monomer domains or combined to form dimers and multimeric domains. For example, a polypeptide that forms a three-dimensional structure that binds to a target molecule is a monomer domain.

According to the present invention the term antibody includes antibodies or antibody derivatives or fragments thereof as well as related molecules of the immunoglobulin superfamily (such as soluble T-cell receptors). Among the antibody fragments are functional equivalents or homologues of antibodies including any polypeptide comprising an immunoglobulin binding domain or a small mutated immunoglobulin domain or peptides mimicking this binding domain together with an Fc region or a region homologous to an Fc region or at least part of it. Chimeric molecules comprising an immunoglobulin binding domain, or equivalents, fused to another polypeptide are included.

Allergens are antigens to which atopic patients respond with allergic reactions.

### BRIEF DESCRIPTION OF THE DRAWINGS

FIG. 1 is a graph showing the results of an experiment in which an autoimmune response was induced in mice using ScFV-1-coil and mAb IV.3.

FIG. 2 is a graph showing IFN-α production by human pDCs after stimulation with CPG-C.

FIG. 3 is another graph showing IFN-α production of human pDCs after stimulation with CPG-C.

FIG. 4 is a graph showing IL-6 and TNFα production of pDCS after stimulation with CPG-C.

FIG. 5 is a graph showing the production of IgG antibodies in response to various immunogens.

FIG. 6 is a graph of T cell proliferation in response to various immunogens.

FIG. 7 is a graph of cytokine production in response to various immunogens.

### DETAILED DESCRIPTION OF THE INVENTION

The invention provides a molecule or a molecule complex being capable of binding to toll-like receptor 9 (TLR9) and Fc gamma receptor RII (CD32) and including at least one epitope of at least one antigen.

In one embodiment of the invention the molecule or molecule complex comprises at least three parts, one part being a structure specifically binding to TLR9 (monovalently, bivalently or multivalently), another part being a structure specifically binding to CD32 (monovalently, bivalently or multivalently) and at least one other part being one or more T cells epitopes of an antigen and/or allergen. The parts may be independent structures which are linked together either by chemical linkages or by genetic fusion or by other (non-covalent) interactions such as ligand-receptor or antibody interactions.

The linkages between the different parts may be different. For example, in one preferred embodiment, the linkage between the parts binding to TLR9 and CD32 is by genetic fusion and the link to at least one of the T cell epitopes is via a receptor/ligand interaction (e.g. biotin-streptavidin). The advantage of such a setup is the flexibility in production. The bispecific (anti-TLR9/anti-CD32), generic part of the molecule complex can be produced in the same way for all patients, selected T cell epitopes are linked to the generic part of the molecule complex according to the need. The selection can be based on disease prevalence or on results of individual specificity tests of patients (specific allergy). The complex formation may be performed centralized or at the bed side or at a physician's office.

Chemical linkage of molecules of the various binding molecules of the same or different chemical class may be achieved by many different techniques yielding either a defined molecular ratio of the various parts of the molecule or molecule complex of the invention. It may also lead to a mixture of molecules with different molecular ratios of the various parts of the molecule or molecule complex of the invention.

The ratio of the various parts of the invention may be an equimolar or non-equimolar. The molecule may be monovalent for binding to TLR9 and/or CD32 and/or T cell epitope(s). It may also be bi-, tri- and multivalent for at least one of the parts of the molecule or the molecule complex. If the binding to TLR9 and/or CD32 is bivalent or of higher valency, the binding specificity may be for one or for more epitopes on CD32 and/or TLR9 respectively.

In another embodiment of the invention the binding specificities of the molecule are overlapping so that one part of the molecule or the molecule complex of the invention is binding to both, TLR9 and CD32. Such a part could be selected for by simultaneous screening of molecules for binding to both CD32 and TLR9 or by engineering of a molecule to bind both, CD32 and TLR9. For example, a protein scaffold can be used for displaying loops to bind CD32 and other loops that bind to TLR9.

In a further embodiment of the invention, a protein scaffold can be used to display structures that bind CD32, structures that bind TLR9 and to display T-cell epitopes.

The specific binding molecules can be natural ligands for CD32 and TLR9 and derivatives thereof. For example, the Fc-part of immunoglobulin is binding to CD32. CpG is a naturally occurring ligand for TLR9.

The specific binding molecules can be peptides. CD32- and TLR9-specific peptides according to the invention can be selected by various methods such as phage display technology or by screening of combinatorial peptide libraries or peptide arrays. The peptides can be selected and used in various formats such as linear, constrained or cyclic peptides, the peptides can be chemically modified for stability and/or specificity.

A specifically binding peptide may also be derived from analysis of interaction of a naturally occurring proteinaceous ligand to TLR9 and CD32 by isolation of the minimal binding site of the ligand.

The specific binding peptides can be used as such in the molecule or the molecule

complex of the invention or used to be incorporated into other structures such as by grafting into protein scaffolds, antibodies and protein domains or chemically coupled to carrier molecules which might be part of the molecule or molecule complex of the invention.

The binding part of the molecules or molecule complex of the invention can be comprised of proteins such as antibodies or antibody fragments (such as Fab, Fv, scFv, dAb, F(ab)₂, minibody, small mutated immunoglobulin domains, soluble T-cell receptor, etc.). Antibodies and antibody fragments and derivatives may be generated and selected for binding to TLR9 and/or CD32 according to known methods such as hybridoma technology, B-cell cloning, phage display, ribosome display or cell surface display of antibody libraries, array screening of variant antibodies.

The binding parts of the molecules or molecule complexes of the invention can be protein domains which occur naturally or domains which are artificially modified. Protein domains or domain derivatives, e.g. domains with mutations such as amino acid substitutions, deletions or insertions or chemically modified domains may be selected for binding to TLR9 and/or CD32 according to known methods (e.g. phage and cell surface display of libraries of domains or domain variants and screening, arrays of variant molecules and screening). The domains include but are not limited to molecules from the following classes:
EGF-like domain, a Kringle-domain, a fibronectin type I domain, a fibronectin type Il domain, a fibronectin type III domain, a PAN domain, a GIa domain, a SRCR domain, a Kunitz/Bovine pancreatic trypsin Inhibitor domain, a Kazal-type serine protease inhibitor domain, a Trefoil (P-type) domain, a von Willebrand factor type C domain, an Anaphylatoxin-like domain, a CUB domain, a thyroglobulin type I repeat, a LDL-receptor class A domain, a Sushi domain, a Link domain, a Thrombospondin type I domain, an immunoglobulin domain, an Immunoglobulin-like domain, a C-type lectin domain, a MAM domain, a von Willebrand factor type A domain, an A-domain, a Somatomedin B domain, a WAP-type four disulfide core domain, an F5/8 type C domain, a Hemopexin domain, an SH2 domain, an SH3 domain, a Laminin-type EGF-like domain, a CTLA-4 domain, a C2 domain.

In a preferred embodiment, the binding part of a molecule or molecule complex of the invention comprises a small mutated immunoglobulin domain (SMID) as described in PCT/EP2006/050059.

The binding part of the molecule or molecule complex of the invention can be nucleic acids such as RNAs or DNAs which can be selected for specific binding to TLR9 and/or CD32 according to known methods such as aptamer screening and in vitro evolution techniques.

It is contemplated that also other molecule classes will be able to show specific binding to TLR9 and or CD32. Libraries of other chemical entities than the ones mentioned above, including carbohydrates, lipids, and small organic molecules, may be screened for specific binding to TLR9 and/or CD32 and may be incorporated into the molecule or molecule complex of the invention.

A preferred embodiment of the invention is a recombinant fusion protein consisting of at least one epitope of at least one antigen, at least one binding site interacting with TLR9 and at least one binding site interacting with CD32. The antigen can be as small as one T cell epitope from one antigen or can be a cocktail or mixture of one or more T cell epitopes from one or more different antigens fused or linked together in a way that allows proper processing and presentation by MHC molecules. The order of the epitopes can be selected according to different criteria such as product stability effective processing, (non-)recognition by preformed antibodies in the treated persons. Generally one will select for a stable molecule which can be efficiently presented by MHC and which will lead to minimal recognition by preformed antibodies.

The invention further comprises the physical coupling of at least one molecule interacting with TLR9, at least one molecule interacting with CD32 and one or more T cell epitopes from one or more antigens linked together in a random form.

Additionally, the invention provides the preparation of a medicament containing the fusion protein according to the invention and its use for treatment of allergies. The medicament can be a vaccine formulation containing the molecule or molecule complex according to the invention, useful esp. for active immunotherapy.

The recombinant production of bispecific binding structures of the molecule or the molecule complex of the invention (i.e. binding to CD32 and to TLR9) can be accomplished in different ways, e.g. by:
Quadroma technology (fused hybridomas) [12; 13];
bispecific scFvs, either as “diabodies” or simply by genetic fusion of different scFvs [14];
single-domain antibodies in which VH recognizes one antigen and VL another one;
chi-bAbs (as described in EP0640130);
small mutated immunoglobulin domains, by including engineered immunoglobulin domains, specifically binding to CD32 and/or to TLR9 in constructs coding either for complete antibodies or for antibody fragments such as Fab (according to PCT/EP2006/050059);
in the context of this invention, binding to CD32 can also be accomplished by monomeric or multimeric immunoglobulin Fc region(s) or a parts thereof especially when the affinity for CD32 of the Fc parts is enhanced, while TLR9 binding is achieved through the normal binding site (VH/VL) of the antibody;
Fc-region(s) of an immunoglobulin or parts thereof, binding to CD32, fused to any other TLR9 specific binding motif;
the Fc part of the above mentioned antibody may be “glyco-engineered” to increase the affinity for human FcγR's [15] engineered scaffolds, specifically binding to TLR9 and/or CD32 of any kind can be used and linked together as needed. These binding scaffolds can be protein domains, fibronectin III, lipocalins, Protein A or α-amylase inhibitor, Ankyrin Repeat Proteins, a C2 domain, an A-domain, an EGFR like domain, a dab, a chi-bAb, CTLA-4, gamma crystalline or any other protein, protein domain or part thereof.

The molecule or molecule complex of the invention consist of one or more epitopes and one or more binding structures, which interact with TLR9, preferably human TLR9 and one or more binding structures, which interact with CD32, preferably human CD32. For easy in vivo testing of the inventive protein the binding structures that recognize human TLR9 and human CD32 may cross react with monkey or mouse TLR9 and monkey or mouse CD32. The selected antigens/allergens may be complete natural/native proteins or parts of these, as long as epitopes which can be presented on MHC class Il molecules and which can be recognized by T cells are present on the sequences present in the molecule or molecule complex. The part(s) of the molecule or molecule complex, which interact with TLR9 and CD32 may be complete or incomplete (modified) antibodies or fragments or derivatives thereof, as long as binding to TLR9 and CD32 is retained.

Alternatively, anti-TLR9 and anti-CD32 antibodies or derivatives or fragments thereof, which still specifically recognize and bind to human TLR9 and CD32 such as expressed by B cells, and dendritic cells can be used.

Alternatively, the antibodies interacting with TLR9 or CD32 are improved antibodies with higher affinity than the original antibodies.

Exemplary antibody molecules are intact immunoglobulin molecules and those portions of an immunoglobulin molecule that contains the paratope, including those portions known as Fab, Fab′, F(ab′)₂, Fc and F(v), dAb.

The antibodies can be IgG, IgM, IgE, IgA or IgD. The molecules interacting with TLR9 or CD32 can be of any origin, preferably of mammalian origin, preferably of human, mouse, camel, dog or cat origin or humanized. Preferably the molecules are antibodies, preferably human or humanized antibodies.

As used herein, if the molecule or molecule complex of the invention is a fusion protein, it can be expressed in host cells which cover any kind of cellular system which can be modified to express the fusion protein. Within the scope of the invention, the term “cells” means individual cells, tissues, organs, insect cells, avian cells, mammalian cells, hybridoma cells, primary cells, continuous cell lines, stem cells and/or genetically engineered cells, such as recombinant cells expressing an antibody according to the invention.

Cells can be bacterial cells, fungal cells yeast cells, insect cells, fish cells and plant cells. Preferably the cells are animal cells, more preferably mammalian cells. These can be for example BSC-1 cells, LLC-MK cells, CV-1 cells, CHO cells, COS cells, PerC6 cells, murine cells, human cells, HeLa cells, 293 cells, VERO cells, MDBK cells, MDCK cells, MDOK cells, CRFK cells, RAF cells, TCMK cells, LLC-PK cells, PK15 cells, WI-38 cells, MRC-5 cells, T-FLY cells, BHK cells, SP2/0, NSO cells or derivatives thereof.

Preferably the binding structures of the molecule or the molecule complex of the invention recognizing TLR9 and CD32 are small mutated immunoglobulin domains, being for example an Fab fragment in which one binding site (either specific for CD32 or for TLR9) is formed by VH/VL, and is combined with a second binding site (either specific for TLR9 or for CD32 respectively) which can be an engineered CL or an engineered CH1, CH2, CH3, CH4, VL or VH domain according to PCT/EP/2006/050059; or a complete antibody in which one binding site is formed by VH/VL, and is combined with a second binding site which can be an engineered CL, CH1, CH2, CH3, CH4, VL or VH domain according to PCT/EP/2006/050059.

According to the invention, the molecule or molecule complex contains at least one structure that specifically binds to CD32.

An anti-CD32 antibody can be derived by known methods (such as hybridoma technology, B-cell cloning and antibody library screening). For selection, cells displaying CD32 in a natural format can be used or a recombinant extracellular part of CD32 can be used or synthetic peptides selected from the CD32 amino acid sequence can be used. Selection criteria are that the binding structure recognizes CD32a. In case also CD32b is recognized it is preferred that the affinity for CD32a≧CD32b.

As an example, the Fab fragment from the anti-CD32 IV.3 antibody derived from the cell line HB-217 can be used. Using the method e.g. described by Orlandi et al 16, the Fab fragment is cloned from the cell line HB-217. Alternatively, other formats such as scFv can be constructed of the known V-gene sequences. However, for optimal combination with an anti-TLR9 antibody or Fab fragment or Fv fragment it is preferred to select specific binders using one or more of the small mutated immunoglobulin domain libraries from CH1, CH2 CH3, CH4, CL, VL or VH.

Selected CH1, CH2, CH3, CH4, CL1 VL or VH domains can then be cloned into the existing sequence of an anti-TLR9 antibody or a Fab or an Fv fragment thereof thus generating a bi-specific antibody or Fab fragment.

The selected CD32 binding entities should preferably have the following characteristics:

1. Interaction with CD32a leads to internalization of the receptor-binding-structure complex, activation of the antigen presenting cell through the ITAM motif in the cytoplasmic tail of the receptor and antigen presentation of the linked/fused T cell epitopes;

2. Interaction with CD32b leads to negative signaling of the receptor through the ITIM motif;

3. Interaction should show cross reactivity between human and monkey CD32 (for testing of efficacy in a relevant in vivo model);

4. Interaction should show cross reactivity with mouse CD32 (for testing in an established in vivo model for allergy);

For obtaining a binding structure that specifically binds to TLR9, several procedures can be used (such as hybridoma technology, B-cell cloning and antibody library screening). For selection, cells expressing TLR9 in a natural format can be used to isolate natural TLR9 or a recombinant TLR9 can be used or synthetic peptides selected from the TLR9 amino acid sequence can be used. Alternatively, purified TLR9 or TLR9 expressed by cell lines can be used. Antibody genes coding for VL and VH respectively can be extracted after selection for binding to TLR9 and be used to design a recombinant antibody or Fab fragment specific for human TLR9. Alternatively, a single-chain Fv can also be made and fused with the anti-CD32 scFv mentioned above. However, for optimal combination with the anti-CD32 antibody or scFv or Fab fragment it is preferred to select specific binders using one or more of the small mutated immunoglobulin domain libraries from CH1, CH2 CH3, CH4, CL, VH or VL. Selected CH1, CH2, CH3, CH4, CL, VL or VH domains can then be cloned into the existing antibody or Fab fragment or scFv of anti-CD32 antibody thus generating a bi-specific Fab fragment. The selected TLR9 binding entities should preferably have the following characteristics:

1. Interaction with TLR9 leads to signal transduction and cytokine production;

2. Interaction may show cross reactivity between human and monkey TLR9 (for testing of efficacy in a relevant in vivo model);

3. Interaction may show cross reactivity with mouse TLR9 (for testing in an established in vivo model for allergy) and CD32.

Of course the fusion protein can similarly be made using the Fab part of an existing aTRL9 monoclonal antibody. Using the method e.g. described by Orlandi et al 16, the Fab fragment is cloned from e.g. clone 26C593 available from Imgenex Corp., as described above for the fab fragment of the aCD32 Ab IV.3. Again for optimal combination with the anti-TLR9 Fab fragment it is best to select specific binders for CD32 using one or more of the small mutated immunoglobulin domain libraries from CH1, CH2, CH3, CH4, CL, VL or VH. Selected CH1, CH2, CH3, CH4, CL, VL or VH domains can then be cloned into the existing Fab fragment of anti-TLR9 antibody thus generating a bi-specific molecule.

Finally, e.g. in the absence of available suitable existing Ab's for both CD32 and TLR9, it is also possible to construct a bi-specific molecule using the small mutated immunoglobulin domain libraries from CH1, CH2 CH3 or CL to select specific binders for both CD32 and TLR9 which are subsequently combined to form new structures existing of at least 1 binding structure specific for CD32 and 1 binding structure specific for TLR9 derived from any of the possible libraries in any of the possible combinations (CH1-CH1 or CH1-CH2 or CH1 CH3 or CH2-CH4, or CH3-CH4, or CH1-CH4 or CH2-CH3 etc.).

Alternatively, a single variable domain of the immunoglobulin superfamily may be selected for binding to TLR9 or CD32 with CDR-loops. The selected binder is then randomized at non-structural loop positions to generate a library of variable domains which is selected for the respective other antigen, i.e. in case of a variable domain binding with CDR loops to TLR9 the selection is for binding to CD32 and vice versa. It is also possible to select a library of a V-domain which contains variations in the CDR loops at the same time as variations in the non-CDR-loops for binding to TLR9 and CD32 sequentially or simultaneously.

Such bispecific V-domains may also be part of antibodies or antibody fragments such as single-chain-Fvs, Fabs or complete antibodies.

Selection of a suitable TLR9 epitope, i.e. sequence 244-256 (SEQ ID No 1) of the mature TLR9 protein in amino acid 1 letter code:

CPRHFP QLHPDTFS
244 250 257

will fulfill criterion 1 and 2 but not 3, whereas sequence 176-191 (SEQ ID No 2) of the mature protein TLR9 in amino acid 1 letter code:

LTHL SLKYNNLTVV PR
176 180 191

and

Sequence 216-240 of the mature protein TLR9 (SEQ ID No 3) in amino acid 1 letter code:

ANLT ALRVLDVGGN CRRCDHAPNP C
216 220 230 240

will fulfill all three criteria and are thus preferred for use in this invention.

The process for producing the molecule or molecule complex is carried out according to known methods, e.g. by using recombinant cloning techniques or by chemical cross linking.

A product as described in this invention can be produced in the following way:

The obtained VH and VL of the anti-CD32 antibody are fused to CH 1 and CL respectively. The CL has previously been engineered using SMID technology (PCT/EP2006/050059) and selected using phage display to bind to TLR9 as described below. CH1 is fused at its C-terminus to a sequence encoding the selected T cell epitopes. These two fusion-protein encoding genes are cloned into an expression vector allowing the expression of two independent genes (or into two independent expression vectors) and are co-expressed in bacteria, yeast or animal cells or any other suitable expression system. Thus, a Fab with the desired characteristics, i.e. binding to CD32, binding to TLR9 and carrying the relevant T-cell epitopes is produced.

Alternative examples applying SMID technology include:
An scFv against TLR9 is derived from a phage display library or from an existing hybridoma, and a CD32 binding molecule is derived from a CH2-CH4, or CH3-CH4, or CH1-CH4 or small mutated immunoglobulin domain library. These two coding sequences are ligated together and a sequence coding for T cell epitopes is attached. The fusion protein is then expressed in bacteria, yeast or animal cells or any other suitable expression system;
Alternatively, TLR9-specificity and CD32-specificity are swapped: An scFv against CD32 is derived e.g. from a phage display library or from an existing hybridoma, and a TLR9-binding molecule is derived from a CH2-CH4, or CH3-CH4, or CH1-CH4 or small mutated immunoglobulin domain library. These two coding sequences are ligated together and a sequence coding for T cell epitopes is attached. The fusion protein is then expressed in bacteria, yeast or animal cells or any other suitable expression system;
VH and VL of an anti-TLR9 antibody are fused to CH 1 and CL respectively. CL has previously been engineered and selected using phage display to bind to CD32 (SMID). CH1 is fused at its C-terminus to a sequence encoding the T cell epitopes. These two fusion-protein encoding genes are cloned into an expression vector allowing the expression of two independent genes (or into two independent expression vectors) and are coexpressed in bacteria, yeast or animal cells or any other suitable expression system, (again, anti-TLR9 and anti-CD32 can be swapped. CH1 and CL can also be swapped);
Heavy and light chain genes of an anti-TLR9 antibody are taken as a whole. In the heavy chain gene, the CH2 (or CH1 or CH3 or CH4) region is replaced by a CH2 (or CH1 or CH3 or CH4 or CL or VH or VL) region which has previously been engineered and selected using phage display to bind to CD32 (small mutated immunoglobulin domain). CH1, CH2, CH3 or CH4 is fused at its C-terminus to a sequence encoding the T cell epitopes. These two genes are again cloned in expression vectors and expressed in animal cells;
2 small mutated immunoglobulin domains, one specific for TLR9, the other specific for CD32 are fused and combined with T-cell epitopes;
1 small mutated immunoglobulin domain with 2 different specificities (TLR9 and CD32) is combined with T-cell epitopes.

Antigens and Epitopes:

The antigens that are part of the molecule or molecule complex according to the invention can be complete allergens, denatured allergens or any antigens that are treated in any possible way to prevent binding to IgE. Such treatment may consist of epitope shielding of the antigenic protein using high affinity IgM, IgD, IgA or IgG antibodies directed to the same epitopes as the patient's IgE antibodies as described by Leroy et al [20]. Such antibodies may also bind close to the IgE specific epitopes thus preventing binding of the IgE antibodies by sterical hindrance.

Allergens are generally defined as antigens to which atopic patients respond with IgE antibody responses subsequently leading to allergic reactions. Antigens used in the molecule or the molecule complex of the invention can be environmental allergens (e.g. house dust mite, birch pollen, grass pollen, cat antigens, cockroach antigens), or food allergens (e.g. cow milk, peanut, shrimp, soya), or a combination of both. Also non relevant antigens such as HSA can be part of the molecule or molecule complex according to the invention. The antigen can be a complete allergen, exemplary an allergen for which patients with atopic dermatitis, allergic asthma, allergic rhinitis or allergic conjunctivitis are allergic. Preferably the allergen use in the molecule or molecule complex according to the invention does not bind to IgE from the patient in need of treatment.

The antigens and/or epitopes used in the invention can be from natural sources or be produced by recombinant technology or be produced synthetically. Antigens and/or epitopes of the invention may contain ligand structures which facilitate incorporation of antigens and/or epitopes into molecule complexes of the invention via ligand/receptor interactions or antibody binding. Antigens and/or epitopes of the invention may contain chemical groups which facilitate covalent linkage of the antigens and/or epitopes to the CD32- and/or TLR9-binding structures of the molecule of the invention.

In one embodiment of the invention the antigens and epitopes of the molecule or molecule complex of the invention may be covalently linked to the CD32 binding structure and/or to the TLR9 binding structure.

In one embodiment antigens and/or epitopes may also be linked by a ligand/receptor interaction such as biotin and avidin to the molecule or molecule complex of the invention. For example, the antigens or epitopes to be used in the molecule of the invention may be produced with biotin or a biotin mimetic attached to it. The CD32 binding structure and/or the TLR9 binding structure may be produced with avidin or another biotin-specific ligand attached to it. After mixing of these molecules with the different attachments, a stable molecule complex is formed according to the invention. Alternatively, an antibody/antigen binding can be used to form a molecule complex of the invention. High affinity interactions are preferred for these embodiments (e.g. high affinity anti-digoxigenin antibody and digoxigenin labeled antigens and/or epitopes).

In one embodiment of the invention the antigens and/or epitopes are genetically fused to the CD32-binding structure and/or to the TLR9-binding structure.

If the molecule of the invention is a fusion protein, the antigen is preferably produced from at least one T-cell epitope-containing DNA-subsequence of an allergen. The T cell epitopes can alternatively be from one or more related and/or unrelated allergens.

Preferably, the T cell epitopes comprise a new protein, which is not as such a naturally existing protein and therefore is not recognized by existing IgE or IgG antibodies in the patient. Therefore, instead of selecting short T cell epitopes which are cut apart and fused together again in a different order, one could also select a larger stretch of T cell epitopes (>28 AA) which are still in their natural order but which have been previously selected not to bind to allergen specific IgE [21].

In principle all known antigens can be used for incorporation into the molecule or molecule complex of the invention to which allergic patients respond with IgE mediated hypersensitivity reactions. The most common environmental allergens in the developed countries are: house dust mite, birch pollen, grass pollen, cat, and cockroach. Each of these allergens has one or more “major allergens” (e.g. house dust mite: major allergen=Der P1; Der F1, birch pollen: major allergen=Bet V1). However, complete antigens, though possible, are not necessary, because the molecule or molecule complex should only induce T cell responses, and T cells respond to small (ca. 12-28 amino acid long) peptides presented in MHC Class Il molecules. The selection of T cell epitopes should be designed in such a way that expression on HLA class Il molecules of possibly all patients is guaranteed. Some HLA class Il molecules are more frequently expressed than others. A good example for such a HLA class Il molecule with wide expression is HLA DPw4, which is expressed on approximately 78% of the Caucasian population [22]. Therefore a selection of T cell epitopes could be included in the molecule or molecule complex for each allergen, thus reducing the size and molecular weight of the complex. If overlapping cross-reactive epitopes between allergens from different genetically related organisms, such as Dermatophagoides pteronyssinus (Der P1) and Dermatophagoides farinae, (Der F1), are present, they are preferred.

To allow for correct antigen processing, DNA coding for stretches slightly longer than the actual T cell epitope should be included in the molecule or molecule complex and/or the epitopes can be separated from each other by introducing stretches of spacer DNA preferably containing (hydrophobic) epitopes recognized by major protein processing enzymes in antigen presenting cells such the asparagine-specific endopeptidase (AEP) or cathepsin S, cathepsin D or cathepsin L [23].

For fusion to the genes coding for the binding structures specific for TLR9 and CD32, preferably short DNA sequences of major allergens are used such as house dust mite major allergen I (Der P1, Der F1), house dust mite major allergen Il (Der P2, Der F2), or birch pollen allergen (Bet V1). These short DNA sequences contain the genetic code for one or more T cell epitopes, which after processing, appear on the surface of antigen presenting cells and therefore induce an immune response in the responding allergen specific T cells. Not only T cell epitopes from Der P1 and Der P2 but also Der P3, Der P4, Der P5, Der P6, Der P7 etc. and Der F3, Der F4, Der F5, Der F6, Der F7 etc. can be used in a molecule or molecule complex of the invention. T cell epitopes from these allergens may be selected by classical epitope mapping using T cell clones [24] or by using modern HLA Class Il predicting software such as the Tepitope program [25; 26]. For the molecule or molecule complexes, which can be formulated as vaccine, it is not necessary to combine T cell epitopes from a single allergen source only; to the contrary it is preferred to include as many T cell epitopes derived from different allergen sources produced by one or many different species, e.g. a combination of allergens from house dust mites and of allergens from grass pollen, cats and/or birch pollen.

As an example for Der P1 the majority of the T cell epitopes can be found in the following sequences 101-143 of the mature protein in amino acid 1 letter code (SEQ ID No 4):

QSCRRPNAQ RFGISNYCQI YPPNANKIRE ALAQPQRYCR HYWT
101 110 120 130 140 143

Especially the amino acid sequence 101-131 contains at least 3 T cell epitopes 24, which bind to a number of HLA class Il molecules in amino acid 1 letter code (SEQ ID No 5):

QSCRRPNAQ RFGISNYCQI YPPNANKIRE AL
101 110 120 131

The sequence 107-119 contains an important T cell epitope that binds to HLA DPw4 as well as HLA DPw5 24. These HLA Class Il molecules are expressed by the majority of the population. The epitope in amino acid 1 letter code (SEQ ID No 6):

NAQ RFGISNYCQI
107 110 119

Other important T cell epitopes which in addition are shared between Der P1 and Der F1 are found in the sequences 20-44 and 203-226 of the mature protein in amino acid 1 letter code:

(SEQ ID No 7)

RTVTPIRMQG GCGSCWAFSG VAATE
20 30 40 44

and

(SEQ ID No 8)

YDGRTII QRDNGYQPNY HAVNIVGY
203 210 220 227

Examples of T cells epitopes shared between Der P2 and Der F2 are found in the sequence 26-44, 89-107 and 102-123

(SEQ ID No 9)

PCII HRGKPFQLEA VFEAN
26 30 40 44

(SEQ ID No 10)

K YTWNVPKIAP KSENVVVT
89 100 107

(SEQ ID No 11)

ENVVVTVK VMGDDGVLAC AIAT
102 110 123 127

From the above mentioned T cell epitopes of Der P1/F1 and Der P2/F2 one can design several functional molecule or molecule complexes, e.g.: By taking from Der P1 the following sequences:

(Sequence A, SEQ ID No 12)

QSCRRPNAQ RFGISNYCQI YPP
101 110 120

(Sequence B, SEQ ID No 13)

CQI YPPNANKIRE AL
117 120 130

(Sequence C, SEQ ID No 14)

IRE ALAQPQRYCR HYWT
127 130 140 143

(Sequence D, SEQ ID No 7)

RTVTPIRMQG GCGSCWAFSG VAATE
20 30 40 44

(Sequence E, SEQ ID No 8)

YDGRTII QRDNGYQPNY HAVNIVGY
203 210 220 227

And from Der P 2

(Sequence F, SEQ ID No 9)

PCII HRGKPFQLEA VFEAN
26 30 40 44

(Sequence G, SEQ ID No 10)

K YTWNVPKIAP KSENVVVT
89 100 107

(Sequence H, SEQ ID No 11)

ENVVVTVK VMGDDGVLAC AIAT
102 110 120 123

One can design a cDNA with the order B,A,E,H,G,C,F,D or H,A,D,C,F,G,E,B, but any possible combination of the selected sequences will do. The preferred order of the epitopes will largely be determined on the basis of expression efficiency of the complete recombinant molecule. Also duplications of sequences are allowed e.g. B,B,A,E,E,G,C,G,F,A,D etc. The T cell epitope part may of course also contain the genetic codes for shorter peptides or longer peptides for more and for fewer peptides, as long as one or more T cell epitopes from one or more different allergens/antigens are included.

Epitopes from other allergens such as Bet V1, Lol P1, FeL d1 with similar characteristics will be preferred for inclusion in the molecule or molecule complex according to the invention.

The invention also concerns a method of treating diseases, especially allergies, which comprises administering to a subject in need of such treatment a prophylactically or therapeutically effective amount of a molecule or molecule complex according to the invention for use as a pharmaceutical, especially as an agent against allergies.

The molecule or molecule complex may be admixed with conventional pharmaceutically acceptable diluents and carriers and, optionally, other excipients and administered parenterally intravenously or enterally, e.g. intramuscularly and subcutaneously. The concentrations of the molecule or molecule complex will, of course, vary depending, for example, on the compound employed, the treatment desired and the nature of the form.

For different indications the appropriate doses will, of course, vary depending upon, for example, the molecule or molecule complex used, the host, the mode of application and the intended indication. However, in general, satisfactory results are indicated to be obtained with 1 to 4 vaccinations in 1-2 years, but if necessary repeated additional vaccination can be done. It is indicated that for these treatments the molecule or molecule complex of the invention may be administered in 2-4 doses and with an application schedule similar as conventionally employed.

It further concerns a molecule or molecule complex according to the invention for use as a pharmaceutical, particularly for use in the treatment and prophylaxis of allergies.

The pharmaceutical composition prepared according to the present invention for use as vaccine formulation can (but does not have to) contain at least one adjuvant commonly used in the formulation of vaccines apart from the molecule or molecule complex. It is possible to enhance the immune response by such adjuvants. As examples of adjuvants, however not being limited to these, the following can be listed: aluminum hydroxide (Alu gel), QS-21, Enhanzyn, derivatives of lipopolysaccharides, Bacillus Calmette Guerin (BCG), liposome preparations, formulations with additional antigens against which the immune system has already produced a strong immune response, such as for example tetanus toxoid, Pseudomonas exotoxin, or constituents of influenza viruses, optionally in a liposome preparation, biological adjuvants such as Granulocyte Macrophage Stimulating Factor (GM-CSF), interleukin 2 (IL-2) or gamma interferon (IFNγ). Aluminum hydroxide is the most preferred vaccine adjuvant.

Summary of a Possible Mode of Action of the Fusion Protein According to the Invention:

The fusion protein according to the present invention, can be formulated in any of the available acceptable pharmaceutical formulations, but is preferably formulated as a vaccine. The aCD32 binding portion of the fusion protein according to the invention selects the relevant cells. Triggering of CD32 on these cells will actively induce internalization of the receptor plus the attached fusion protein and by doing so facilitates the interaction of the TLR9 binding portion of the fusion protein with the TLR9, which is expressed within the cytoplasm of the relevant antigen presenting cells [10; 11].

As a consequence of the CD32 mediated internalization, the subsequent processing and presentation of the selected T cell epitopes on MHC Class Il molecules, combined with the specific activation of cytoplasmic TRL9 in the antigen presenting cells, allergen specific T cells will be (re-)programmed to become Th1 memory cells. These allergen specific Th1 memory cells at a later time point will induce allergen specific IgG production when encountering the same epitopes derived from the natural allergens presented by naturally exposed allergen specific B cells. These TM cells thus are necessary for rebalancing the immune system from IgE to IgG dominated antibody production.

### EXAMPLES

The following examples shall explain the present invention in more detail, without, however, restricting it.

### Example 1

Panning of the human CL-phage library on a TLR-9 peptide e.g. sequence 216-240 of the mature protein TLR9 (SEQ ID No 3) in amino acid 1 letter code

ANLT ALRVLDVGGN CRRCDHAPNP C
216 220 230 240

3 panning rounds shall be performed according to standard protocols. Briefly, the following method can be applied. Maxisorp 96-well plates (Nunc) are coated with the (synthetic) peptide representing part of the sequence of the TLR-9. For coating the peptides in the wells, 200 μl of the following solution are added per well: 0.1 M

Na-carbonate buffer, pH 9.6, with the following concentrations of dissolved peptide:

1st panning round: 1 mg/ml TLR-9 peptide

2nd panning round: 500 μg/ml TLR-9 peptide

3rd panning round: 100 μg/ml TLR-9 peptide

Incubation is for 1 hour at 37° C., followed by blocking with 2% dry milk (M-PBS) with 200 μl per well for 1 hour at room temperature. The surface display phage library is then allowed to react with the bound peptide by adding 100 μl phage suspension and 100 μl 4% dry milk (M-PBS), followed by incubation for 45 minutes with shaking and for 90 minutes without shaking at room temperature. Unbound phage particles are washed away as follows. After the 1st panning round: 10×300 μl T-PBS, 5×300 μl PBS; after the 2nd panning round: 15×300 μl T-PBS, 10×300 μl PBS; after the 3rd panning round: 20×300 μl T-PBS, 20×300 μl PBS. Elution of bound phage particles is performed by adding 200 μl per well of 0.1 M glycine, pH 2.2, and incubation with shaking for 30 minutes at room temperature. Subsequently, the phage suspension is neutralized by addition of 60 μl 2M Tris-Base, followed by infection into E. coli TG1 cells by mixing 10 ml exponentially growing culture with 0.5 ml eluted phage and incubation for 30 minutes at 37° C. Finally, infected bacteria are plated on TYE medium with 1% glucose and 100 μg/ml Ampicillin, and incubated at 30° C. overnight.

### Example 2

Cloning of Selected Clones of Human CL Mutants Selected Against TLR-9 for Soluble Expression

Phagemid DNA from the phage selected through the 3 panning rounds is isolated with a midi-prep. DNA encoding mutated CL-regions is batch-amplified by PCR and cloned Ncol-Notl into the vector pNOTBAD/Myc-His, which is the E. coli expression vector pBAD/Myc-His (Invitrogen) with an inserted Notl restriction site to facilitate cloning. Ligated constructs are transformed into E. coli LMG194 cells (Invitrogen) with electroporation, and grown at 30° C. on TYE medium with 1% glucose and ampicillin overnight. Selected clones are inoculated into 200 μl 2×YT medium with ampicillin, grown overnight at 30° C., and induced by adding L-arabinose to an end concentration of 0.1%. After expression at 16° C. overnight, the cells are harvested by centrifugation and treated with 100 μl Na-borate buffer, pH 8.0, at 4° C. overnight for preparation of periplasmic extracts. 50 μl of the periplasmic extracts were used in ELISA (see below).

### Example 3: ELISA of Human CL Mutants Selected Against TLR-9

Selected clones are assayed for specific binding to the TLR-9 peptide by ELISA.

Coating: Microtiter plate (NUNC, Maxisorp), 100 μl per well, 20 μg TLR-9 peptide/ml 0.1 M Na-carbonate buffer, pH 9.6, 1 h at 37° C.

Wash: 3×200 μl PBS

Blocking: 1% BSA-PBS, 1 h at RT

Wash: 3×200 μl PBS

Periplasmic extract binding: 50 μl periplasmic extract

50 μl 2% BSA-PBS, at room temperature overnight

Wash: 3×200 μl PBS

1st antibody: anti-His4 (Qiagen), 1:1000 in 1% BSA-PBS, 90 min at RT, 100 μl per well

Wash: 3×200 μl PBS

2nd antibody: goat anti mouse*HRP (SIGMA), 1:1000 in 1% BSA-PBS, 90 min at RT, 100 μl per well

Wash: 3×200 μl PBS

Detection: 3 mg/ml OPD in Na-citrate/phosphate buffer, pH 4.5, 0.4 μl 30% H2O2

Stopping: 100 ml 3M H2SO4

Absorbance read: 492/620 nm

Clones that give a high signal in this first, preliminary ELISA are cultured in a 20-ml volume at the same conditions as described above. Their periplasmic extracts are isolated in 1/20 of the culture volume as described above and tested with ELISA (as described above) for confirmation.

### Example 4: Cloning of the Anti-CD32 Variable Domains from HB-217

mRNA is isolated from the cell line HB-217 (ATCC, antiCD32 antibody IV.3) and is used to prepare cDNA according to established routine protocols. The cDNA is further used as a template to amplify the regions of the genes coding for the of the light and the heavy chain of the Fab fragment of antibody IV.3 respectively. Upstream PCR primers, which prime from the 5′ end of the variable regions, used for this amplification are derived from the published sequences of mouse variable regions (IMGT, the international ImMunoGeneTics information System®). Degenerate primers and/or mixtures of different primers are used as upstream primers. Downstream primers are designed such as to prime from the 31 end of the CL or the CH 1 domains respectively.

In a next step, the CL domain of the antibody IV.3 is removed and replaced by a selected CL domain modified by SMID technology which has binding affinity to TLR9, and which is selected as described above in examples 1-3. For this replacement, overlapping PCR can be used according to standard protocols. Alternatively, for joining VL to the SMID modified CL a uniquely cutting restriction site can be used which is either naturally occurring in the sequence or which is artificially introduced by site directed mutagenesis (as a silent mutation which does not change the amino acid sequence). For example, a BstAPI site can be generated in the hinge region between VL and CL by changing the sequence from:

(SEQ ID No 65)

K R A D A A P T V S I F

(SEQ ID No 66)

AAACGGGCTGATGCTGCACCAACTGTATCCATCTTC
to:

(SEQ ID No 65)

K R A D A A P T V S I F

(SEQ ID No 15)

AAACGGGCAGATGCTGCACCAACTGTATCCATCTTC

the newly created BstAPI site is highlighted in the above sequence. The new sequence is introduced in the coding regions by amplifying the VL part and the CL part respectively with appropriately designed PCR primers, cutting the PCR products with BstAPI, ligating them, and amplifying the complete resulting ligation product with PCR primers as used initially for amplifying the original light chain part of the Fab fragment.

For expression of the modified Fab fragment, the genes coding for the heavy and the light chains are subsequently cloned in appropriate expression vectors, or together in one expression vector which allows the expression of two independent genes. As an expression system, bacteria, yeast, animal cells or any other suitable expression system can be used. For this example here, expression from one vector in the methylotrophic yeast Pichia pastoris will be shown.

The light chain part of the modified PCR fragment is cloned EcoRI/Kpnl in the Pichia pastoris expression vector pPICZalphaA in the correct reading frame such as to fuse it functionally with the alpha-factor secretion signal sequence provided by the vector. Similarly, the heavy chain part of the Fab fragment is cloned in pPICZalphaA. In order to prepare the inserts for this cloning procedure, appropriately designed PCR primers are used which attach the needed restriction sites to the genes. At the 31 ends of both coding regions, a stop codon has to be inserted and provided by the PCR primers as well. The light chain expression cassette is then cut out from the vector with restriction enzymes BgIII and BamHI, and the ends of the DNA are made blunt by treatment with Klenow fragment of DNA polymerase. The vector containing the inserted heavy chain part of the Fab is opened by a partial digest with restriction enzyme BgIII, the DNA is made blunt by treatment with Klenow fragment of DNA polymerase, and the expression cassette coding for the light chain part is inserted. The partial digest of the heavy chain vector is necessary since the inserted heavy chain gene contains a BgIII site. For screening of the final construct, care has to be taken that this internal BgIII site has remained intact. The final construct has one Pmel site which is used for linearizing the construct prior to transformation into Pichia pastoris. This linearization is advantageous for efficient integration of the expression vector in the host genome by homologous recombination. Pichia pastoris is transformed with the linearized expression vector using electroporation, transformed clones are selected with the antibiotic Zeocin for which the vector confers resistance, and supernatants of randomly picked clones are screened for expression of the Fab construct after induction of expression with methanol. For screening, e.g. a Fab-specific ELISA can be used. Production of the recombinant protein is achieved by culturing the transformed selected Pichia clone in a larger scale, preferable in shake flasks or in a fermenter, inducing expression by addition of methanol and purifying the recombinant protein by a chromatographic method. For these latter steps, routine protocols are used.

### Example 5: Cloning of the Der P1I/F1 and Der P2/F2 Derived T Cell Epitopes

The combination of the selected T cell epitopes formed by sequences B,A,E,H,G,C,F,D looks as follows (SEQ ID No 16):

```
CQIYPPNANKIREAL QSCRRPNAQRFGISNYCQIYPP YDGRTIIQRDNGYQPNYHAVNI
(Seq: B) (Seq. A) (Seq. E)
VGY ENVVVTVKVMGDDGVLACAIAT KYTWNVPKIAPKSENVVVT IREALAQPQRYCRH
        (Seq. H) (Seq. G) (Seg. C)
YWT PCIIHRGKPFQLEAVFEAN RTVTPIRMQGGCGSCWAFSGVAATE
        (Seq. F) (Seq. D)
```

In order to construct a synthetic gene coding for this amino acid sequence, in silico reverse translation can be used. Computer programs are available for this purpose, such as e.g. DNAWORKS, in order to clone the synthetic gene coding for the epitopes in frame with the gene coding for the heavy chain part of the framework, two restriction sites are selected which cut neither on this coding region nor on the vector pPICZalphaA. For example, AccIII and SpeI can be used for this purpose. These two restriction sites are attached to the gene coding for the heavy chain part of the Fab by using appropriately designed PCR primers for the cloning procedure as described above. Furthermore, care has to be taken not to have a stop codon at the end of the coding region of the heavy chain part of the Fab, as the stop codon will be provided at the 3′ end of the synthetic gene coding for the epitopes. Again, this construct with the two additional restriction sites located at its 3′ end is cloned EcoRI/Kpnl in the Pichia pastoris expression vector pPICZalphaA. The construct is then opened with the restriction enzymes AccIII and SpeI and the insert coding the epitopes in inserted. This insert is generated as follows:

The chosen amino acid sequence:

(SEQ ID No. 16)
CQIYPPNANKIREAL QSCRRPNAQRFGISNYCQIYPP YDGRTIIQRDNGYQPNYHAVN
IVGY ENVVVTVKMGDDGVLACAIAT KYTWNVPKIAPKSENVVVT IREALAQPQRYC
RHYWT PCIIHRGKPFQLEAVFEAN RTVTPIRMQGGCGSCWAFSGVAATE

together with the chosen restriction sites, in this example AccIII at the 5′ end and SpeI at the 3′ end are used as input in the publicly available computer program DNAWORKS. In addition, a stop codon is added between the end of the epitope sequence and the SpeI site.

The parameters which the program uses for designing the oligonucleotides are left at the proposed standard values, and the program is instructed to avoid the sequences of the restriction sites which are necessary for the cloning and transformation steps, such as AccIII, SpeI and PmeI.

AccIII: tccgga

SpeI: actagt

PmeI: gtttaaac

DNAWORKS generates a set of oligonucleotides which are overlapping and which represent both strands of the desired coding regions.

For example, the following set of 24 oligonucleotides is generated, from which the synthetic gene coding for the allergen epitopes is generated:

| | | | |
|---|---|---|---|
| 1 | TCCGGATGCCAAATTTACCCGCCAAACG | 28 | (SEQ ID No 17) |
| 2 | AGCCTCTCTGATCTTGTTCGCGTTTGGCGGGTAAATTTGG | 40 | (SEQ ID No 18) |
| 3 | CGAACAAGATCAGAGAGGCTTTGCAATCTTGCAGGAGGCC | 40 | (SEQ ID No 19) |
| 4 | TATGCCGAATCTCTGCGCATTGGGCCTCCTGCAAGATTGC | 40 | (SEQ ID No 20) |
| 5 | GCGCAGAGATTCGGCATATCCAACTACTGCCAGATCTACC | 40 | (SEQ ID No 21) |
| 6 | GTACGCCCATCGTATGGGGGGTAGATCTGGCAGTAGTTGG | 40 | (SEQ ID No 22) |
| 7 | CCCATACGATGGGCGTACAATCATACAGCGTGATAACGGC | 40 | (SEQ ID No 23) |
| 8 | GCGTGGTAGTTAGGCTGATAGCCGTTATCACGCTGTATGA | 40 | (SEQ ID No 24) |
| 9 | TATCAGCCTAACTACCACGCCGTGAACATCGTCGGCTACG | 40 | (SEQ ID No 25) |
| 10 | TCACAGTAACCACGACATTCTCGTAGCCGACGATGTTCAC | 40 | (SEQ ID No 26) |
| 11 | AGAATGTCGTGGTTACTGTGAAGGTAATGGGCGATGACGG | 40 | (SEQ ID No 27) |
| 12 | AGCTATGGCGCAAGCTAGAACCCCGTCATCGCCCATTACC | 40 | (SEQ ID No 28) |
| 13 | TCTAGCTTGCGCCATAGCTACCAAGTACACTTGGAACGTA | 40 | (SEQ ID No 29) |
| 14 | TTTTCGGCGCAATTTTGGGTACGTTCCAAGTGTACTTGGT | 40 | (SEQ ID No 30) |
| 15 | CCCAAAATTGCGCCGAAAAGTGAAAACGTCGTAGTGACCA | 40 | (SEQ ID No 31) |
| 16 | TGAGCCAATGCCTCCCTTATGGTCACTACGACGTTTTCAC | 40 | (SEQ ID No 32) |
| 17 | AGGGAGGCATTGGCTCAACCTCAAAGATACTGCAGACACT | 40 | (SEQ ID No 33) |
| 18 | TTATGCAGGGCGTCCAGTAGTGTCTGCAGTATCTTTGAGG | 40 | (SEQ ID No 34) |
| 19 | ACTGGACGCCCTGCATAATCCACCGTGGTAAACCCTTTCA | 40 | (SEQ ID No 35) |
| 20 | CTTCGAACACTGCCTCAAGTTGAAAGGGTTTACCACGGTG | 40 | (SEQ ID No 36) |
| 21 | ACTTGAGGCAGTGTTCGAAGCTAACAGGACGGTAACGCCA | 40 | (SEQ ID No 37) |
| 22 | CCGCACCCACCTTGCATACGAATTGGCGTTACCGTCCTGT | 40 | (SEQ ID No 38) |
| 23 | TGCAAGGTGGGTGCGGGTCTTGTTGGGCTTTTTCTGGTGT | 40 | (SEQ ID No 39) |
| 24 | ACTAGTTTATTCAGTAGCAGCCACACCAGAAAAAGCCCAACA | 42 | (SEQ ID No 40) |

These 24 oligonucleotides are dissolved, mixed together, boiled for several minutes and then cooled down to room temperature slowly to allow annealing. In a subsequent PCR steps using large amounts of the two bordering primers (primers #1 and #24), the annealed gene is amplified, the PCR product is then cleaved with the chosen restriction enzymes (AccIII and SpeI in this example), and cloned into the expression vector as described above, which contains as an insert the gene coding for the heavy chain part of the modified Fab. Preparation of the final expression vector containing both chains, transformation of Pichia pastoris, selection of clones and screening for producing clones is done as described above. Expression and purification of the recombinant protein is performed by following standard protocols.

### Example 6: Fusion of VH and VL of the Anti-CD32 Antibody IV.3 Fusion with Anti-TLR9 CH3 Domains (SMIDS)

All molecular modeling was done with Swiss-PdbViewer 3.7.

As a homology model for a mouse Fab fragment, the structure file 2BRR.pdb from the Protein Data Bank is used, and 1OQO.pdb is used as a source for the structure of a human IgG CH3 domain.

Molecular models of VH and VL of the IV.3 antibody are made with the “first approach mode” of the Swissmodel system using the amino acid sequences of VH and VL respectively.

Using the “magic fit” function of the Swiss-PdbViewer, two copies of the CH3 domain structure from 1OQO.pdb are fitted onto the CH1 and the CL domain respectively of 2BRR.pdb. Subsequently, the molecular models of the IV.3 VH and VL respectively are fitted (again using “magic fit”) onto VH and VL of 2BRR.pdb.

For construction of a Fab-like protein in which CH 1 and CL are both replaced by a CH3 domain, it is necessary to decide at which point the sequence of VH should be ended and connected to the sequence of CH3, and at which point the sequence of VL should be ended and connected to the sequence of CH3. For both constructs, a point is chosen at which the main chain of the superimposed structures and models (see above) shows an optimal overlap.

For the light chain, it was found that the sequence up to Ala114 (numbering from 2BRR.pdb) will be used an connected to Pro343 (numbering from 1OQO.pdb) of the CH3 domain. The point of connection between these two sequences therefore reads as follows (VL part is underlined): - - - Lys112-Arg113-Ala114-Pro343-Arg344-Glu345 - - - _{—}

In order to allow joining of the two coding sequences using restriction enzyme sites and DNA ligation, the sequence near the point of connection is changed by silent mutation to introduce a unique Xhol site (ctcgag, underlined) as follows:

(SEQ ID No 41)

K R A P R E

(SEQ ID No 42)

AAACGGGCTCCTCGAGAA

For later insertion of the allergen epitopes, an AscI site (ggcgcgcc) is introduced just before the stop codon of the construct plus an extra base for maintenance of the reading frame:

ggg cgc gcc

### Gly Arg Ala

Furthermore, for cloning into the expression vector pPICZalphaA (Pichia pastoris expression system, Invitrogen), an EcoRI site (gaattc) is added to the 5′-end (N-terminus) and a Kpnl site (ggtacc) to the 3′-end (C-terminus) of the construct.

The CH3 domain to be fused to VH and VL respectively selected as part of the construct can be a wild type human IgG CH3 domain which can serve as a negative control, or a CH3 domain previously engineered by SMID technology and selected to bind specifically to TLR9. In this example here, the sequence of clone A23, which binds specifically to TLR9 and which was described in the patent application PCT/EP2006/050059 is fused to both, VH and VL.

Therefore, the complete sequence of the VL-CH3 fusion protein has the following amino acid sequence (VL part is underlined), (SEQ ID No 43):

DIVMTQAAPS VPVTPGESVS ISCRSSKSLL HTNGNTYLHW FLQRPGQSPQ
LLIYRMSVLA SGVPDRFSGS GSGTAFTLSI SRVEAEDVGV FYCMQHLEYP
LTFGAGTKLE LKRAPREPQV YTLPPSRDEL GIAQVSLTCL VKGFYPSDIA
VEWESNGQPE NNYKTTPPVL DSDGSFFLYS KLTVLGRRWT LGNVFSCSVM
HEALHNHYTQ KSLSLSPGK&

Nucleic acid sequence of the VL-CH3 fusion protein (restriction sites are underlined), (SEQ ID No 44):

gaattcGACA TTGTGATGAC CCAGGCTGCA CCCTCTGTAC CTGTCACTCC
TGGAGAGTCA GTATCCATCT CCTGCAGGTC TAGTAAGAGT CTCCTGCATA
CTAATGGCAA CAGTTACTTG CATTGGTTCC TACAGAGGCC AGGCCAGTCT
CCTCAGCTCC TGATATATCG GATGTCCGTC CTTGCCTCAG GAGTCCCAGA
CAGGTTCAGT GGCAGTGGGT CAGGAACTGC TTTCACACTG AGCATCAGTA
GAGTGGAGGC TGAGGATGTG GGTGTTTTTT ACTGTATGCA ACATCTAGAA
TATCCGCTCA CGTTCGGTGC TGGGACCAAG CTGGAACTGA AACGGGCTCC
TCGAGAACCA CAGGTGTACA CCCTGCCCCC ATCCCGGGAC GAGCTCGGCA
TCGCGCAAGT CAGCCTGACC TGCCTGGTCA AAGGCTTCTA TCCCAGCGAC
ATCGCCGTGG AGTGGGAGAG CAACGGGCAG CCGGAGAACA ACTACAAGAC
CACGCCTCCC GTGCTGGACT CCGACGGCTC TTTCTTCCTC TACAGCAAGC
TTACCGTGTT GGGCCGCAGG TGGACCCTGG GGAACGTCTT CTCATGCTCC
GTGATGCATG AGGCTCTGCA CAACCACTAC ACACAGAAGA GCCTCTCCCT
GTCTCCGGGT AAATGAgggc gcgccggtac c

For the heavy chain, it was found that the sequence up to ThM 23 (numbering from 2BRR.pdb) should be used an connected to Arg344 (numbering from 1OQO.pdb) of the CH3 domain. The point of connection between these two sequences therefore reads as follows (VH part is underlined): - - - -Ala121-Lys122-Thr123-Arg344-Glu345-Pro346 - - -

In order to allow joining of the two coding sequences using restriction enzyme sites and DNA ligation, the sequence near the point of connection was changed by silent mutation to introduce a unique Xhol site (ctcgag, underlined) as follows:

(SEQ ID No 45)

A K T R E P

(SEQ ID No 46)

GCCAAAACTCGAGAACCA

Furthermore, for cloning into the expression vector pPICZalphaA (Pichia pastoris expression system, Invitrogen), an EcoRI site (gaattc) is added to the 5′-end (N-terminus) and an Xbal site (tctaga) to the 3′-end (C-terminus) of the construct. No stop codon is added to this sequence and the Xbal site is placed in the correct reading frame so as to fuse the construct to the Hexa-His-tag provided by the vector for later purification of the protein using immobilized metal affinity chromatography.

Therefore, the complete sequence of the VH-CH3 fusion protein has the following amino acid sequence (VH part is underlined), (SEQ ID No 47):

EVQLQQSGPE LKKPGETVKI SCKASGYTFT NYGMNWVKQA PGKGLKWMGW
LNTYTGESIY PDDFKGRFAF SSETSASTAY LQINNLKNED MATYFCARGD
YGYDDPLDYW GQGTSVTVSS AKTREPQVYT LPPSRDELGI AQVSLTCLVK
GFYPSDIAVE WESNGQPENN YKTTPPVLDS DGSFFLYSKL TVLGRRWTLG
NVFSCSVMHE ALHNHYTQKS LSLSPGKSLE QKLISEEDLN SAVDHHHHHH&

Nucleic acid sequence of the VH-CH3 fusion protein (restriction sites are underlined), (SEQ ID No 48):

GAATTCGAGG TTCAGCTTCA GCAGTCTGGA CCTGAGCTGA AGAAGCCTGG
AGAGACAGTC AAGATCTCCT GCAAGGCTTC TGGGTATACC TTCACAAACT
ATGGAATGAA CTGGGTGAAG CAGGCTCCAG GAAAGGGTTT AAAGTGGATG
GGCTGGTTAA ACACCTACAC TGGAGAGTCA ATATATCCTG ATGACTTCAA
GGGACGGTTT GCCTTCTCTT CGGAAACCTC TGCCAGCACT GCCTATTTGC
AGATCAACAA CCTCAAAAAT GAGGACATGG CTAGATATTT CTGTGCAAGA
GGGGACTATG GTTACGACGA CCCTTTGGAC TACTGGGGTC AAGGAACCTC
AGTCACCGTC TCCTCAGCCA AAACTCGAGA ACCACAGGTG TACACCCTGC
CCCCATCCCG GGACGAGCTC GGCATCGCGC AAGTCAGCCT GACCTGCCTG
GTCAAAGGCT TCTATCCCAG CGACATCGCC GTGGAGTGGG AGAGCAACGG
GCAGCCGGAG AACAACTACA AGACCACGCC TCCCGTGCTG GACTCCGACG
GCTCTTTCTT CCTCTACAGC AAGCTTACCG TGTTGGGCCG CAGGTGGACC
CTGGGGAACG TCTTCTCATG CTCCGTGATG CATGAGGCTC TGCACAACCA
CTACACACAG AAGAGCCTCT CCCTGTCTCC GGGTAAATCT CTAGAACAAA
AACTCATCTC AGAAGAGGAT CTGAATAGCG CCGTCGACCA TCATCATCAT
CATCATTGA

Detailed Cloning Plan

Heavy Chain:

The VH region of antibody IV.3 is PCR-amplified with primers 4.3HupEco and 4.3HdownXho, and subsequently digested with EcoRI and Xhol. The CH3 SMID-engineered clone A23 is PCR-amplified with primers CH3upXhoA and CH3XBA2 and subsequently digested with Xhol and Xbal. The VH sequence and the CH3 sequence are ligated together via the Xhol site and then ligated into pPICZalphaA (Invitrogen), which was previously digested with EcoRI and Xbal. The resulting vector is named pPICHA23.

Primer List:

4.3HUPECO

(SEQ ID No 49)

cagagaattc gaggttcagc ttcagcagtc
4.3HDOWNXHO

(SEQ ID No 50)

gatgctcgag ttttggctga ggagacggtg
CH3UPXHOA

(SEQ ID No 51)

aaaactcgag aaccacaggt gtacaccctg cc
CH3XBA2

(SEQ ID No 52)

actgatctag acctttaccc ggagacaggg agag

Light Chain:

The VL region of antibody IV.3 is PCR-amplified with primers 4.3LupEco and 4.3LdownXho, and subsequently digested with EcoRI and Xhol. The CH3 SMID-engineered clone A23 is PCR-amplified with primers CH3upXhoB and CH3StopKpn and subsequently digested with Xhol and Kpnl. The VL sequence and the CH3 sequence are ligated together via the Xhol site and then ligated into pPICZalphaA (Invitrogen), which was previously digested with EcoRI and Kpnl. The resulting vector is named pPICLA23.

Primer List:

4.3LUPECO

(SEQ ID No 53)

gatagaattc gacattgtga tgacccaggc tg
4.3LDOWNXHO

(SEQ ID No 54)

attactcgag gagcccgttt cagttccagc t
CH3UPXHOB

(SEQ ID No 55)

gctcctcgag aaccacaggt gtacaccctg cc
CH3STOPKPN

(SEQ ID No 56)

acgtggtacc tcaggcgcgc cctttacccg gagacaggga gag

Combination of the Two Expression Cassettes in One Vector

The light chain cassette is cut out with BgIII (pos.1) and BamHI (pos. 2319) from pPICLA23 (4235 bp), and the 2319 bp fragment is purified via preparative gel electrophoresis. The 1916 bp fragment is discarded. The vector pPICHA23 (4219 bp) is digested with BamHI, and the previously purified 2319 bp fragment from pPICLA23 is inserted. The resulting Pichia pastoris expression vector, which carries two expression cassettes, one for the VL-CH3 fusion protein and on for the VH-CH3 fusion protein is screened so that both inserts that have same direction of transcription. The resulting vector pPICHLA23 (6537 bp) is then linearized before transformation into Pichia pastoris e.g. with BamHI or with BssSI, transformed into Pichia pastoris by electroporation, and positive transformants are selected with Zeocin. Several clones are screened for expression of the recombinant protein. A clone is then selected for large scale production, and the recombinant fusion protein is purified by immobilized-metal-affinity chromatography using standard procedures. All Pichia manipulation, culturing and expression is done by following standard protocols (Invitrogen).

Insertion of allergen epitopes into the vector pPICHLA23 and expression of the recombinant fusion protein

The sequence encoding the allergen epitopes as described in example 5 is inserted into the vector pPICHLA23 as follows:

The vector is digested with AscI (4174-4182) which leads to its linearization. In this AscI site, the DNA sequence encoding the allergen epitopes is inserted. The sequence encoding the allergen epitopes is amplified with primers EpiTLRI and EpiTLR2 in order to attach AscI sites to both ends of the sequence.

Primer List

EPiTLR1

(SEQ ID No 57)

TAAAGGGCGC GCCTCCGGAT GCCAAATTTA CC
EpiTLR2

(SEQ ID No 58)

TACCTCAGGC GCGCCTTATT CAGTAGCAGC CACAC

The resulting PCR product is digested with AscI and ligated into the previously digested vector. The resulting vector is named pHLA23EP (7046 bp). Pichia transformation, expression and purification of the recombinant fusion protein is performed as described above for the construct that has no epitopes inserted.

VL of Antibody IV.3

Amino Acid Sequence:

(SEQ ID No 59)

DIVMTQAAPS VPVTPGESVS ISCRSSKSLL HTNGNTYLHW
FLQRPGQSPQ LLIYRMSVLA SGVPDRFSGS GSGTAFTLSI
SRVEAEDVGV EYCMQHLEYP LTFGAGTKLE LKRA

Nucleic Acid Sequence:

(SEQ ID No 60)

GACATTGTGA TGACCCAGGC TGCACCCTCT GTACCTGTCA
CTCCTGGAGA GTCAGTATCC ATCTCCTGCA GGTCTAGTAA
GAGTCTCCTG CATACTAATG GCAACACTTA CTTGCATTGG
TTCCTACAGA GGCCAGGCCA GTCTCCTCAG CTCCTGATAT
ATCGGATGTC CGTCCTTGCC TCAGGAGTGC CAGACAGGTT
CAGTGGCAGT GGGTCAGGAA CTGCTTTCAC ACTGAGCATC
AGTAGAGTGG AGGCTGAGGA TGTGGGTGTT TTTTACTGTA
TGCAACATCT AGAATATCCG CTCACGTTCG GTGCTGGGAC
CAAGCTGGAA CTGAAACGGG CT

VH of Antibody IV.3

Amino Acid Sequence:

(SEQ ID No 61)

EVQLQQSGPE LKKPGETVKI SCKASGYTFT NYGMNWVKQA
PGKGLKWMGW LNTYTGESIY PDDFKGRFAF SSETSASTAY
LQINNLKNED MATYFCARGD YGYDDPLDYW GQGTSVTVSS
AKT

Nucleic Acid Sequence:

(SEQ ID No 62)

GAGGTTCAGC TTCAGCAGTC TGGACCTGAG CTGAAGAAGC
CTGGAGAGAC AGTCAAGATC TGCTGCAAGG CTTCTGGGTA
TACCTTCACA AACTATGGAA TGAACTGGGT GAAGCAGGCT
CCAGGAAAGG GTTTAAAGTG GATGGGCTGG TTAAACACCT
ACACTGGAGA GTCAATATAT CCTGATGACT TCAAGGGACG
GTTTGCCTTC TCTTCGGAAA CCTCTGCCAG CACTGCCTAT
TTGCAGATCA ACAACCTCAA AAATGAGGAC ATGGCTACAT
ATTTCTGTGC AAGAGGGGAC TATGGTTACG ACGACCCTTT
GGACTACTGG GGTCAAGGAA CCTCAGTCAC CGTCTCCTGA
GCCAAAACA

Final Expression Vector pPICHCLA23.seq.

(SEQ ID No 63) containing TLR9 and CD32 binding regions (6537 bp):

| | |
|---|---|
| 1 | agatctaaca tccaaagacg aaaggttgaa tgaaaccttt ttgccatccg acatccacag |
| 61 | gtccattctc acacataagt gccaaacgca acaggagggg atacactagc agcagaccgt |
| 121 | tgcaaacgca ggacctccac tcctcttctc ctcaacaccc acttttgcca tcgaaaaacc |
| 181 | agcccagtta ttgggcttga ttggagctcg ctcattccaa ttccttctat taggctacta |
| 241 | acaccatgac tttattagcc tgtctatcct ggcccccctg gcgaggttca tgtttgttta |
| 301 | tttccgaatg caacaagctc cgcattacac ccgaacatca ctccagatga gggctttctg |
| 361 | agtgtggggt caaatagttt catgttcccc aaatggccca aaactgacag tttaaacgct |
| 421 | gtcttggaac ctaatatgac aaaagcgtga tctcatccaa gatgaactaa gtttggttcg |
| 481 | ttgaaatgct aacggccagt tggtcaaaaa gaaacttcca aaagtcggca taccgtttgt |
| 541 | cttgtttggt attgattgac gaatgctcaa aaataatctc attaatgctt agcgcagtct |
| 601 | ctctatcgct tctgaacccc ggtgcacctg tgccgaaacg caaatgggga aacacccgct |
| 661 | ttttggatga ttatgcattg tctccacatt gtatgcttcc aagattctgg tgggaatact |
| 721 | gctgatagcc taacgttcat gatcaaaatt taactgttct aacccctact tgacagcaat |
| 781 | atataaacag aaggaagctg ccctgtctta aacctttttt tttatcatca ttattagctt |
| 841 | actttcataa ttgcgactgg ttccaattga caagcttttg attttaacga cttttaacga |
| 901 | caacttgaga agatcaaaaa acaactaatt attcgaaacg atgagatttc cttcaatttt |
| 961 | tactgctgtt ttattcgcag catcctccgc attagctgct ccagtcaaca ctacaacaga |
| 1021 | agatgaaacg gcacaaattc cggctgaagc tgtcatcggt tactcagatt tagaagggga |
| 1081 | tttcgatgtt gctgttttgc cattttccaa cagcacaaat aacgggttat tgtttataaa |
| 1141 | tactactatt gccagcattg ctgctaaaga agaaggggta tctctcgaga aaagagaggc |
| 1201 | tgaagctgaa ttcgaggttc agcttcagca gtctggacct gagctgaaga agcctggaga |
| 1261 | gacagtcaag atctcctgca aggcttctgg gtataccttc acaaactatg gaatgaactg |
| 1321 | ggtgaagcag gctccaggaa agggtttaaa gtggatgggc tggttaaaca cctacactgg |
| 1381 | agagtcaata tatcctgatg acttcaaggg acggtttgcc ttctcttcgg aaacctctgc |
| 1441 | cagcactgcc tatttgcaga tcaacaacct caaaaatgag gacatggcta catatttctg |
| 1501 | tgcaagaggg gactatggtt acgacgaccc tttggactac tggggtcaag gaacctcagt |
| 1561 | caccgtctcc tcagccaaaa ctcgagaacc acaggtgtac accctgcccc catcccggga |
| 1621 | tgagctgggc atcgcgcaag tcagcctgac ctgcctggtc aaaggcttct atcccagcga |
| 1681 | catcgccgtg gagtgggaga gcaacgggca gccggagaac aactacaaga ccacgcctcc |
| 1741 | cgtgctggac tccgacggct ctttcttcct ctacagcaag cttaccgtgt tgggccgcag |
| 1801 | gtggaccctg gggaacgtct tctcatgctc cgtgatgcat gaggctctgc acaaccacta |
| 1861 | cacgcagaag agcctctccc tgtctccggg taaatctcta gaacaaaaac tcatctcaga |
| 1921 | agaggatctg aatagcgccg tcgaccatca tcatcatcat cattgagttt gtagccttag |
| 1981 | acatgactgt tcctcagttc aagttgggca cttacgagaa gaccggtctt gctagattct |
| 2041 | aatcaagagg atgtcagaat gccatttgcc tgagagatgc aggcttcatt tttgatactt |
| 2101 | ttttatttgt aacctatata gtataggatt ttttttgtca ttttgtttct tctcgtacga |
| 2161 | gcttgctcct gatcagccta tctcgcagct gatgaatatc ttgtggtagg ggtttgggaa |
| 2221 | aatcattcga gtttgatgtt tttcttggta tttcccactc ctcttcagag tacagaagat |
| 2281 | taagtgagac cttcgtttgt gcagatccaa catccaaaga cgaaaggttg aatgaaacct |
| 2341 | ttttgccatc cgacatccac aggtccattc tcacacataa gtgccaaacg caacaggagg |
| 2401 | ggatacacta gcagcagacc gttgcaaacg caggacctcc actcctcttc tcctcaacac |
| 2461 | ccacttttgc catcgaaaaa ccagcccagt tattgggctt gattggagct cgctcattcc |
| 2521 | aattccttct attaggctac taacaccatg actttattag cctgtctatc ctggcccccc |
| 2581 | tggcgaggtt catgtttgtt tatttccgaa tgcaacaagc tccgcattac acccgaacat |
| 2641 | cactccagat gagggctttc tgagtgtggg gtcaaatagt ttcatgttcc ccaaatggcc |
| 2701 | caaaactgac agtttaaacg ctgtcttgga acctaatatg acaaaagcgt gatctcatcc |
| 2761 | aagatgaact aagtttggtt cgttgaaatg ctaacggcca gttggtcaaa aagaaacttc |
| 2821 | caaaagtcgg cataccgttt gtcttgtttg gtattgattg acgaatgctc aaaaataatc |
| 2881 | tcattaatgc ttagcgcagt ctctctatcg cttctgaacc ccggtgcacc tgtgccgaaa |
| 2941 | cgcaaatggg gaaacacccg ctttttggat gattatgcat tgtctccaca ttgtatgctt |
| 3001 | ccaagattct ggtgggaata ctgctgatag cctaacgttc atgatcaaaa tttaactgtt |
| 3061 | ctaaccccta cttgacagca atatataaac agaaggaagc tgccctgtct taaacctttt |
| 3121 | tttttatcat cattattagc ttactttcat aattgcgact ggttccaatt gacaagcttt |
| 3181 | tgattttaac gacttttaac gacaacttga gaagatcaaa aaacaactaa ttattcgaaa |
| 3241 | cgatgagatt tccttcaatt tttactgctg ttttattcgc agcatcctcc gcattagctg |
| 3301 | ctccagtcaa cactacaaca gaagatgaaa cggcacaaat tccggctgaa gctgtcatcg |
| 3361 | gttactcaga tttagaaggg gatttcgatg ttgctgtttt gccattttcc aacagcacaa |
| 3421 | ataacgggtt attgtttata aatactacta ttgccagcat tgctgctaaa gaagaagggg |
| 3481 | tatctctcga gaaaagagag gctgaagctg aattcgacat tgtgatgacc caggctgcac |
| 3541 | cctctgtacc tgtcactcct ggagagtcag tatccatctc ctgcaggtct agtaagagtc |
| 3601 | tcctgcatac taatggcaac acttacttgc attggttcct acagaggcca ggccagtctc |
| 3661 | ctcagctcct gatatatcgg atgtccgtcc ttgcctcagg agtcccagac aggttcagtg |
| 3721 | gcagtgggtc aggaactgct ttcacactga gcatcagtag agtggaggct gaggatgtgg |
| 3781 | gtgtttttta ctgtatgcaa catctagaat atccgctcac gttcggtgct gggaccaagc |
| 3841 | tggaactgaa acgggctcct cgagaaccac aggtgtacac cctgccccca tcccgggatg |
| 3901 | agctgggcat cgcgcaagtc agcctgacct gcctggtcaa aggcttctat cccagcgaca |
| 3961 | tcgccgtgga gtgggagagc aacgggcagc cggagaacaa ctacaagacc acgcctcccg |
| 4021 | tgctggactc cgacggctct ttcttcctct acagcaagct taccgtgttg ggccgcaggt |
| 4081 | ggaccctggg gaacgtcttc tcatgctccg tgatgcatga ggctctgcac aaccactaca |
| 4141 | cgcagaagag cctctccctg tctccgggta aagggcgcgc ctgaggtacc tcgagccgcg |
| 4201 | gcggccgcca gctttctaga acaaaaactc atctcagaag aggatctgaa tagcgccgtc |
| 4261 | gaccatcatc atcatcatca ttgagtttgt agccttagac atgactgttc ctcagttcaa |
| 4321 | gttgggcact tacgagaaga ccggtcttgc tagattctaa tcaagaggat gtcagaatgc |
| 4381 | catttgcctg agagatgcag gcttcatttt tgatactttt ttatttgtaa cctatatagt |
| 4441 | ataggatttt ttttgtcatt ttgtttcttc tcgtacgagc ttgctcctga tcagcctatc |
| 4501 | tcgcagctga tgaatatctt gtggtagggg tttgggaaaa tcattcgagt ttgatgtttt |
| 4561 | tcttggtatt tcccactcct cttcagagta cagaagatta agtgagacct tcgtttgtgc |
| 4621 | ggatccccca cacaccatag cttcaaaatg tttctactcc ttttttactc ttccagattt |
| 4681 | tctcggactc cgcgcatcgc cgtaccactt caaaacaccc aagcacagca tactaaattt |
| 4741 | tccctctttc ttcctctagg gtgtcgttaa ttacccgtac taaaggtttg gaaaagaaaa |
| 4801 | aagagaccgc ctcgtttctt tttcttcgtc gaaaaaggca ataaaaattt ttatcacgtt |
| 4861 | tctttttctt gaaatttttt tttttagttt ttttctcttt cagtgacctc cattgatatt |
| 4921 | taagttaata aacggtcttc aatttctcaa gtttcagttt catttttctt gttctattac |
| 4981 | aacttttttt acttcttgtt cattagaaag aaagcatagc aatctaatct aaggggcggt |
| 5041 | gttgacaatt aatcatcggc atagtatatc ggcatagtat aatacgacaa ggtgaggaac |
| 5101 | taaaccatgg ccaagttgac cagtgccgtt ccggtgctca ccgcgcgcga cgtcgccgga |
| 5161 | gcggtcgagt tctggaccga ccggctcggg ttctcccggg acttcgtgga ggacgacttc |
| 5221 | gccggtgtgg tccgggacga cgtgaccctg ttcatcagcg cggtccagga ccaggtggtg |
| 5281 | ccggacaaca ccctggcctg ggtgtgggtg cgcggcctgg acgagctgta cgccgagtgg |
| 5341 | tcggaggtcg tgtccacgaa cttccgggac gcctccgggc cggccatgac cgagatcggc |
| 5401 | gagcagccgt gggggcggga gttcgccctg cgcgacccgg ccggcaactg cgtgcacttc |
| 5461 | gtggccgagg agcaggactg acacgtccga cggcggccca cgggtcccag gcctcggaga |
| 5521 | tccgtccccc ttttcctttg tcgatatcat gtaattagtt atgtcacgct tacattcacg |
| 5581 | ccctcccccc acatccgctc taaccgaaaa ggaaggagtt agacaacctg aagtctaggt |
| 5641 | ccctatttat ttttttatag ttatgttagt attaagaacg ttatttatat ttcaaatttt |
| 5701 | tctttttttt ctgtacagac gcgtgtacgc atgtaacatt atactgaaaa ccttgcttga |
| 5761 | gaaggttttg ggacgctcga aggctttaat ttgcaagctg gagaccaaca tgtgagcaaa |
| 5821 | aggccagcaa aaggccagga accgtaaaaa ggccgcgttg ctggcgtttt tccataggct |
| 5881 | ccgcccccct gacgagcatc acaaaaatcg acgctcaagt cagaggtggc gaaacccgac |
| 5941 | aggactataa agataccagg cgtttccccc tggaagctcc ctcgtgcgct ctcctgttcc |
| 6001 | gaccctgccg cttaccggat acctgtccgc ctttctccct tcgggaagcg tggcgctttc |
| 6061 | tcaatgctca cgctgtaggt atctcagttc ggtgtaggtc gttcgctcca agctgggctg |
| 6121 | tgtgcacgaa ccccccgttc agcccgaccg ctgcgcctta tccggtaact atcgtcttga |
| 6181 | gtccaacccg gtaagacacg acttatcgcc actggcagca gccactggta acaggattag |
| 6241 | cagagcgagg tatgtaggcg gtgctacaga gttcttgaag tggtggccta actacggcta |
| 6301 | cactagaagg acagtatttg gtatctgcgc tctgctgaag ccagttacct tcggaaaaag |
| 6361 | agttggtagc tcttgatccg gcaaacaaac caccgctggt agcggtggtt tttttgtttg |
| 6421 | caagcagcag attacgcgca gaaaaaaagg atctcaagaa gatcctttga tcttttctac |
| 6481 | ggggtctgac gctcagtgga acgaaaactc acgttaaggg attttggtca tgagatc |

Final Expression Vector pHLA23EP.seq

(SEQ ID No 64) containing TLR9 and CD32 binding regions and epitope sequence (see SEQ ID No 16) (7046 bp):

| | |
|---|---|
| 1 | agatctaaca tccaaagacg aaaggttgaa tgaaaccttt ttgccatccg acatccacag |
| 61 | gtccattctc acacataagt gccaaacgca acaggagggg atacactagc agcagaccgt |
| 121 | tgcaaacgca ggacctccac tcctcttctc ctcaacaccc acttttgcca tcgaaaaacc |
| 181 | agcccagtta ttgggcttga ttggagctcg ctcattccaa ttccttctat taggctacta |
| 241 | acaccatgac tttattagcc tgtctatcct ggcccccctg gcgaggttca tgtttgttta |
| 301 | tttccgaatg caacaagctc cgcattacac ccgaacatca ctccagatga gggctttctg |
| 361 | agtgtggggt caaatagttt catgttcccc aaatggccca aaactgacag tttaaacgct |
| 421 | gtcttggaac ctaatatgac aaaagcgtga tctcatccaa gatgaactaa gtttggttcg |
| 481 | ttgaaatgct aacggccagt tggtcaaaaa gaaacttcca aaagtcggca taccgtttgt |
| 541 | cttgtttggt attgattgac gaatgctcaa aaataatctc attaatgctt agcgcagtct |
| 601 | ctctatcgct tctgaacccc ggtgcacctg tgccgaaacg caaatgggga aacacccgct |
| 661 | ttttggatga ttatgcattg tctccacatt gtatgcttcc aagattctgg tgggaatact |
| 721 | gctgatagcc taacgttcat gatcaaaatt taactgttct aacccctact tgacagcaat |
| 781 | atataaacag aaggaagctg ccctgtctta aacctttttt tttatcatca ttattagctt |
| 841 | actttcataa ttgcgactgg ttccaattga caagcttttg attttaacga cttttaacga |
| 901 | caacttgaga agatcaaaaa acaactaatt attcgaaacg atgagatttc cttcaatttt |
| 961 | tactgctgtt ttattcgcag catcctccgc attagctgct ccagtcaaca ctacaacaga |
| 1021 | agatgaaacg gcacaaattc cggctgaagc tgtcatcggt tactcagatt tagaagggga |
| 1081 | tttcgatgtt gctgttttgc cattttccaa cagcacaaat aacgggttat tgtttataaa |
| 1141 | tactactatt gccagcattg ctgctaaaga agaaggggta tctctcgaga aaagagaggc |
| 1201 | tgaagctgaa ttcgaggttc agcttcagca gtctggacct gagctgaaga agcctggaga |
| 1261 | gacagtcaag atctcctgca aggcttctgg gtataccttc acaaactatg gaatgaactg |
| 1321 | ggtgaagcag gctccaggaa agggtttaaa gtggatgggc tggttaaaca cctacactgg |
| 1381 | agagtcaata tatcctgatg acttcaaggg acggtttgcc ttctcttcgg aaacctctgc |
| 1441 | cagcactgcc tatttgcaga tcaacaacct caaaaatgag gacatggcta catatttctg |
| 1501 | tgcaagaggg gactatggtt acgacgaccc tttggactac tggggtcaag gaacctcagt |
| 1561 | caccgtctcc tcagccaaaa ctcgagaacc acaggtgtac accctgcccc catcccggga |
| 1621 | tgagctgggc atcgcgcaag tcagcctgac ctgcctggtc aaaggcttct atcccagcga |
| 1681 | catcgccgtg gagtgggaga gcaacgggca gccggagaac aactacaaga ccacgcctcc |
| 1741 | cgtgctggac tccgacggct ctttcttcct ctacagcaag cttaccgtgt tgggccgcag |
| 1801 | gtggaccctg gggaacgtct tctcatgctc cgtgatgcat gaggctctgc acaaccacta |
| 1861 | cacgcagaag agcctctccc tgtctccggg taaatctcta gaacaaaaac tcatctcaga |
| 1921 | agaggatctg aatagcgccg tcgaccatca tcatcatcat cattgagttt gtagccttag |
| 1981 | acatgactgt tcctcagttc aagttgggca cttacgagaa gaccggtctt gctagattct |
| 2041 | aatcaagagg atgtcagaat gccatttgcc tgagagatgc aggcttcatt tttgatactt |
| 2101 | ttttatttgt aacctatata gtataggatt ttttttgtca ttttgtttct tctcgtacga |
| 2161 | gcttgctcct gatcagccta tctcgcagct gatgaatatc ttgtggtagg ggtttgggaa |
| 2221 | aatcattcga gtttgatgtt tttcttggta tttcccactc ctcttcagag tacagaagat |
| 2281 | taagtgagac cttcgtttgt gcagatccaa catccaaaga cgaaaggttg aatgaaacct |
| 2341 | ttttgccatc cgacatccac aggtccattc tcacacataa gtgccaaacg caacaggagg |
| 2401 | ggatacacta gcagcagacc gttgcaaacg caggacctcc actcctcttc tcctcaacac |
| 2461 | ccacttttgc catcgaaaaa ccagcccagt tattgggctt gattggagct cgctcattcc |
| 2521 | aattccttct attaggctac taacaccatg actttattag cctgtctatc ctggcccccc |
| 2581 | tggcgaggtt catgtttgtt tatttccgaa tgcaacaagc tccgcattac acccgaacat |
| 2641 | cactccagat gagggctttc tgagtgtggg gtcaaatagt ttcatgttcc ccaaatggcc |
| 2701 | caaaactgac agtttaaacg ctgtcttgga acctaatatg acaaaagcgt gatctcatcc |
| 2761 | aagatgaact aagtttggtt cgttgaaatg ctaacggcca gttggtcaaa aagaaacttc |
| 2821 | caaaagtcgg cataccgttt gtcttgtttg gtattgattg acgaatgctc aaaaataatc |
| 2881 | tcattaatgc ttagcgcagt ctctctatcg cttctgaacc ccggtgcacc tgtgccgaaa |
| 2941 | cgcaaatggg gaaacacccg ctttttggat gattatgcat tgtctccaca ttgtatgctt |
| 3001 | ccaagattct ggtgggaata ctgctgatag cctaacgttc atgatcaaaa tttaactgtt |
| 3061 | ctaaccccta cttgacagca atatataaac agaaggaagc tgccctgtct taaacctttt |
| 3121 | tttttatcat cattattagc ttactttcat aattgcgact ggttccaatt gacaagcttt |
| 3181 | tgattttaac gacttttaac gacaacttga gaagatcaaa aaacaactaa ttattcgaaa |
| 3241 | cgatgagatt tccttcaatt tttactgctg ttttattcgc agcatcctcc gcattagctg |
| 3301 | ctccagtcaa cactacaaca gaagatgaaa cggcacaaat tccggctgaa gctgtcatcg |
| 3361 | gttactcaga tttagaaggg gatttcgatg ttgctgtttt gccattttcc aacagcacaa |
| 3421 | ataacgggtt attgtttata aatactacta ttgccagcat tgctgctaaa gaagaagggg |
| 3481 | tatctctcga gaaaagagag gctgaagctg aattcgacat tgtgatgacc caggctgcac |
| 3541 | cctctgtacc tgtcactcct ggagagtcag tatccatctc ctgcaggtct agtaagagtc |
| 3601 | tcctgcatac taatggcaac acttacttgc attggttcct acagaggcca ggccagtctc |
| 3661 | ctcagctcct gatatatcgg atgtccgtcc ttgcctcagg agtcccagac aggttcagtg |
| 3721 | gcagtgggtc aggaactgct ttcacactga gcatcagtag agtggaggct gaggatgtgg |
| 3781 | gtgtttttta ctgtatgcaa catctagaat atccgctcac gttcggtgct gggaccaagc |
| 3841 | tggaactgaa acgggctcct cgagaaccac aggtgtacac cctgccccca tcccgggatg |
| 3901 | agctgggcat cgcgcaagtc agcctgacct gcctggtcaa aggcttctat cccagcgaca |
| 3961 | tcgccgtgga gtgggagagc aacgggcagc cggagaacaa ctacaagacc acgcctcccg |
| 4021 | tgctggactc cgacggctct ttcttcctct acagcaagct taccgtgttg ggccgcaggt |
| 4081 | ggaccctggg gaacgtcttc tcatgctccg tgatgcatga ggctctgcac aaccactaca |
| 4141 | cgcagaagag cctctccctg tctccgggta aagggcgcgc ctccggatgc caaatttacc |
| 4201 | cgccaaacgc gaacaagatc agagaggctt tgcaatcttg caggaggccc aatgcgcaga |
| 4261 | gattcggcat atccaactac tgccagatct accccccata cgatgggcgt acaatcatac |
| 4321 | agcgtgataa cggctatcag cctaactacc acgccgtgaa catcgtcggc tacgagaatg |
| 4381 | tcgtggttac tgtgaaggta atgggcgatg acggggttct agcttgcgcc atagctacca |
| 4441 | agtacacttg gaacgtaccc aaaattgcgc cgaaaagtga aaacgtcgta gtgaccataa |
| 4501 | gggaggcatt ggctcaacct caaagatact gcagacacta ctggacgccc tgcataatcc |
| 4561 | accgtggtaa accctttcaa cttgaggcag tgttcgaagc taacaggacg gtaacgccaa |
| 4621 | ttcgtatgca aggtgggtgc gggtcttgtt gggctttttc tggtgtggct gctactgaat |
| 4681 | aaggcgcgcc tgaggtacct cgagccgcgg cggccgccag ctttctagaa caaaaactca |
| 4741 | tctcagaaga ggatctgaat agcgccgtcg accatcatca tcatcatcat tgagtttgta |
| 4801 | gccttagaca tgactgttcc tcagttcaag ttgggcactt acgagaagac cggtcttgct |
| 4861 | agattctaat caagaggatg tcagaatgcc atttgcctga gagatgcagg cttcattttt |
| 4921 | gatacttttt tatttgtaac ctatatagta taggattttt tttgtcattt tgtttcttct |
| 4981 | cgtacgagct tgctcctgat cagcctatct cgcagctgat gaatatcttg tggtaggggt |
| 5041 | ttgggaaaat cattcgagtt tgatgttttt cttggtattt cccactcctc ttcagagtac |
| 5101 | agaagattaa gtgagacctt cgtttgtgcg gatcccccac acaccatagc ttcaaaatgt |
| 5161 | ttctactcct tttttactct tccagatttt ctcggactcc gcgcatcgcc gtaccacttc |
| 5221 | aaaacaccca agcacagcat actaaatttt ccctctttct tcctctaggg tgtcgttaat |
| 5281 | tacccgtact aaaggtttgg aaaagaaaaa agagaccgcc tcgtttcttt ttcttcgtcg |
| 5341 | aaaaaggcaa taaaaatttt tatcacgttt ctttttcttg aaattttttt ttttagtttt |
| 5401 | tttctctttc agtgacctcc attgatattt aagttaataa acggtcttca atttctcaag |
| 5461 | tttcagtttc atttttcttg ttctattaca acttttttta cttcttgttc attagaaaga |
| 5521 | aagcatagca atctaatcta aggggcggtg ttgacaatta atcatcggca tagtatatcg |
| 5581 | gcatagtata atacgacaag gtgaggaact aaaccatggc caagttgacc agtgccgttc |
| 5641 | cggtgctcac cgcgcgcgac gtcgccggag cggtcgagtt ctggaccgac cggctcgggt |
| 5701 | tctcccggga cttcgtggag gacgacttcg ccggtgtggt ccgggacgac gtgaccctgt |
| 5761 | tcatcagcgc ggtccaggac caggtggtgc cggacaacac cctggcctgg gtgtgggtgc |
| 5821 | gcggcctgga cgagctgtac gccgagtggt cggaggtcgt gtccacgaac ttccgggacg |
| 5881 | cctccgggcc ggccatgacc gagatcggcg agcagccgtg ggggcgggag ttcgccctgc |
| 5941 | gcgacccggc cggcaactgc gtgcacttcg tggccgagga gcaggactga cacgtccgac |
| 6001 | ggcggcccac gggtcccagg cctcggagat ccgtccccct tttcctttgt cgatatcatg |
| 6061 | taattagtta tgtcacgctt acattcacgc cctcccccca catccgctct aaccgaaaag |
| 6121 | gaaggagtta gacaacctga agtctaggtc cctatttatt tttttatagt tatgttagta |
| 6181 | ttaagaacgt tatttatatt tcaaattttt cttttttttc tgtacagacg cgtgtacgca |
| 6241 | tgtaacatta tactgaaaac cttgcttgag aaggttttgg gacgctcgaa ggctttaatt |
| 6301 | tgcaagctgg agaccaacat gtgagcaaaa ggccagcaaa aggccaggaa ccgtaaaaag |
| 6361 | gccgcgttgc tggcgttttt ccataggctc cgcccccctg acgagcatca caaaaatcga |
| 6421 | cgctcaagtc agaggtggcg aaacccgaca ggactataaa gataccaggc gtttccccct |
| 6481 | ggaagctccc tcgtgcgctc tcctgttccg accctgccgc ttaccggata cctgtccgcc |
| 6541 | tttctccctt cgggaagcgt ggcgctttct caatgctcac gctgtaggta tctcagttcg |
| 6601 | gtgtaggtcg ttcgctccaa gctgggctgt gtgcacgaac cccccgttca gcccgaccgc |
| 6661 | tgcgccttat ccggtaacta tcgtcttgag tccaacccgg taagacacga cttatcgcca |
| 6721 | ctggcagcag ccactggtaa caggattagc agagcgaggt atgtaggcgg tgctacagag |
| 6781 | ttcttgaagt ggtggcctaa ctacggctac actagaagga cagtatttgg tatctgcgct |
| 6841 | ctgctgaagc cagttacctt cggaaaaaga gttggtagct cttgatccgg caaacaaacc |
| 6901 | accgctggta gcggtggttt ttttgtttgc aagcagcaga ttacgcgcag aaaaaaagga |
| 6961 | tctcaagaag atcctttgat cttttctacg gggtctgacg ctcagtggaa cgaaaactca |
| 7021 | cgttaaggga ttttggtcat gagatc |

In the present application, all temperatures are in degrees Celsius. The following abbreviations are used:

CD32=FcγRII

TLR9=Toll like receptor 9

Der P1=Dermatophagoides pteronissyus major allergen 1

Der P2=Dermatophagoides pteronissyus major allergen 2

Der F1=Dermatophagoides farinae major allergen 1

### Example 7: Exemplary Binders

7.1. CD32 Binding Region, Herein Also Called CD32 Binder

The term “CD32 binding region” or “anti-CD32 moiety” as used herein shall mean a ligand specifically binding to the cellular target CD32, either CD32a, CD32b or both, CD32a and CD32b. The moiety can be any binding structure, such as derived from proteins, polypeptides or peptides, including antibodies and antibody fragments or composite molecules with a binding part. The binding part of the molecules or molecule complex of the invention can be comprised of proteins such as antibodies or antibody fragments, such as Fab, Fv, VH/VL, scFv, dAb, F(ab)₂, minibody, small mutated immunoglobulin domains, or other biological binders, such as soluble T-cell receptor, Darpins, etc. Antibodies and antibody fragments and derivatives may be generated and selected for binding to CD32 according to known methods such as hybridoma technology, B-cell cloning, phage display, ribosome display or cell surface display of antibody libraries, array screening of variant antibodies. Exemplary anti-CD32 moieties are scFv derived from the anti CD32 monoclonal antibody AT-10, IV.3, 2E6 or any other aCD32 monoclonal antibody.

A preferred CD32 binding region is an anti-CD32 antibody or derived from an anti-CD32 antibody, derived from an IgG1 Fc fragment, or a peptide specifically binding to CD32.

Specifically, the CD32 antibody is selected from the group consisting of a full-length antibody, an scFv or a VH/VL dimer, specifically binding the CD32.

A specific CD32 peptide is a CD32a peptide with the sequence of SEQ ID 66.

CD32a Binders:

Antibody specifically binding to CD32a: mAb IV.3 (Stuart et al. (1987) J. Exp. Med. 166: 1668)

ScFV derived from mAb IV.3 (VH-linker-VL):

(SEQ ID 67)

EVQLQQSGPELKKPGETVKISCKASGYTFTNYGMNWVKQAPGKGLKWM
GWLNTYTGESIYPDDFKGRFAFSSETSASTAYLQINNLKNEDMATYFCAR
GDYGYDDPLDYWGQGTSVTVSSGGGGSGGGGSGGGGSDIVMTQAAPS
VPVTPGESVSISCRSSKSLLHTNGNTYLHWFLQRPGQSPQLLIYRMSV
LASGVPDRFSGSGSGTAFTLSISRVEAEDVGVFYCMQHLEYPLTFGAG
TKLELKGSI

Underlined: VH domain
Bold: HL domain
Normal type set. Flexible linker (maybe any linker)

Anti-CD32a Peptide: Berntzen et al. (J. Biol. Chem. (2009) 284: 1126-1135):

(SEQ ID 68):
ADGAWAWVWLTETAVGAAK

Group CD32a+b Binders:

Antibody specifically binding to CD32a and CD32b: mAb AT-10 (AbD Serotec) ScFV derived from mAb AT-10 (VH-linker-VL) (SEQ ID 69):

EVKLEESGGGLVQPGGSMKLSCVASGFTFSYYWMNWVRQSPEKGLEWV
AEIRLKSNNYATHYAESVKGRFTISRDDSKNNVYLQMNNLRAEDTGIYYC
NRRDEYYAMDYWGQGTSVSVSSGGGGSGGGGSGGGGSDIVLTQSPGSL
AVSLGQRATISCRASESVDNFGISFMNWFQQKPGQPPRLLIYGASNQG
SGVPARFSGSGSGTDFSLNIHPVEEDDAAMYFCQQSKEVPWTFGGGT
KLEIKGSI

Underlined: VH domain
Bold: HL domain
Normal type set. Flexible linker (maybe any linker)

### IgG1 Fc Fragment (CH2-CH3 Domain) (SEQ ID 70):

(PKSCDKTHTCPPCP)PELLGGPSVFLFPPKPKDTLMISRTPEVTCVVVD
VSHEDPEVKFNWYVDGVEVHNAKTKPREEQYNSTYRVVSVLTVLHQDWLN
GKEYKCKVSNKALPAPIEKTISKAKGQPREPQVYTLPPSRDELTKNQVSL
TCLVKGFYPSDIAVEWESNGQPENNYKTTPPVLDSDGSFFLYSKLTVDKS
RWQQGNVFSCSVMHEALHNHYTQKSLSLSPGK

Between ( ) is hinge region may be omitted
Underlined: CH2 domain
Bold: CH3 domain

7.2 TLR9 Binding Region, Herein Also Called TLR9 Binder or TLR9 Ligand

The term “TLR9 binding region”, “TLR9 binder” or “TLR9 ligand” as used herein is understood in the following way.

Toll-like receptor 9 (TLR9) recognizes unmethylated bacterial CpG DNA and initiates a signaling cascade leading to the production of proinflammatory cytokines. There are numerous structures or sequences that have been shown to act as a ligand of TLR9, i.e. bind to this receptor and thereby either activate (stimulate, upregulate) or de-activate (downregulate) TLR9. For instance, microbial DNA or synthetic DNA, e.g. synthetic CpG ODN may stimulate TLR9 with variations in the number and location of CpG dimers, as well as the precise base sequences flanking the CpG dimers. Synthetic CpG ODN differ from microbial DNA in that they have a partially or completely phosphorothioated backbone instead of the typical phosphodiester backbone and may or may not have a poly G tail at the 3′ end, 5′ end, or both.

The TLR9 ligand typically is coupled to the directed adjuvant component of the present vaccine by chemical coupling e.g. using the commercially available KIT from Solulink. A peptidic TLR9 ligand may be coupled using standard peptide chemistry or may be integrated using recombinant DNA technology.

Exemplary TLR9 ligands are ODN 2216 (group 1), ODN 2006/ODN 2007 (group2) and CpG-M362 (group 3).

The function of a TLR9 ligand or agonist or antagonist may be determined in a suitable assay, e.g. in the following way: pDCs are purified from blood of a healthy donor as described by Tel et al (Immunobiology 2012 October; 217(10):1017-24) and subsequently incubated with the appropriate concentration of the TLR9 ligand. After 24 h IFNα is measured in the supernatant using standard ELISA protocols. For determination of the maturation state of the cells, pDCs are stained for expression of CD80 CD83 or CD86 using standard FACS procedures with commercially available specific antibodies before and after the incubation with the TLR9 ligand.

The number of reactive T cells that are activated upon exposure to the vaccine according to the invention may be determined by a number of methods including ELISPOT, FACS analysis, cytokine release, or T cell proliferation assays.

TLR9 ligand is a TLR9 agonist selected from the group consisting of CpG class A, in particular CpG-A (D)2 oligodeoxynucleotides (ODN), also known as “D”-type ODN. Such TLR9 agonists induce a strong IFNa induction and minimal maturation of dendritic cells, and are herein called “group 1” TLR9 ligand.

According to another aspect of the invention, TLR9 ligand is a TLR9 agonist selected from the group consisting of CpG class B, in particular CpG-B (K)2 oligodeoxynucleotides (ODN), also known as “K”-type ODN. Such TLR9 agonists induce a weak IFNa induction and maturation of dendritic cells, and are herein called “group 2” TLR9 ligand.

According to another aspect of the invention, said TLR9 ligand specifically is a TLR9 agonist selected from the group consisting of CpG class C, also known as CpG-C2; 3 oligodeoxynucleotides (ODN). Such TLR9 agonists induce IFNa and maturation of immature dendritic cells, and are herein called “group 3” TLR9 ligand.

According to another aspect of the invention, TLR9 ligand is a TLR9 antagonist selected from the group consisting of inhibitory ODNs oligodeoxynucleotides (sometimes called inhibitory CPGs), e.g. those which contain the inhibitory motif consisting of CCx(not-C)(not-C)xxGGG (x=any base)6. Specific inhibitory ODNs have proven not to induce IFNa and not to induce maturation of dendritic cells, also blocking activation through an agonist of TLR9.

Such TLR9 agonist or antagonist can be determined in a suitable cell based assay, which measures stable expression of either of IFNa, or at least one of the markers CD80, CD83 and CD86, which reflect the maturation of immature dendritic cells (DC). For this purpose plasmacytoid dendritic cells (pDCs) are purified from blood of a healthy donor as described by Tel et al (see above) and subsequently incubated with the appropriate concentration of the TLR9 ligand. After 24 h IFNa is measured in the supernatant using standard ELISA protocols. For determination of the maturation state of the cells, pDCs are stained for expression of CD80 CD83 or CD86 using standard FACS procedures with commercially available specific antibodies before and after the incubation with the TLR9 ligand.

The induction of IFNa may be determined by the level of IFNa expression and the respective increase with respect to a reference level. The increase relative to non-stimulated cells may be compared to the induction levels induced by established references for each type of CpG as defined by group 1, 2 or 3 TLR9 ligand and is typically between 30% and 300% of the respective reference, preferably at least 100%, more preferably at least 120%, at least 150%, at least 200% or at least 250%.

The maturation of immature dendritic cells may be determined by the level of expression of any of the markers CD80, CD83 and CD86. The respective increase relative to non-stimulated cells may be compared to the induction levels induced by established references for each type of CpG as defined by group 1, 2 or 3 TLR9 ligand and is typically between 30% and 300% of the respective reference, preferably at least 100%, more preferably at least 120%, at least 150%, at least 200% or at least 250%.

Specifically, the TLR9 agonist of group 1 and 3 would result in an increased IFNa expression and a TRL9 agonist of group 2 and 3 would lead to an increased expression of any of the DC maturation factors CD80, CD83 and CD86. The TLR9 antagonist would result in a reduced IFNa expression and a reduced expression of any of the DC maturation factors CD80, CD83 and CD86, even in the presence of a TLR9 agonist of either group 1-3.

### CpG Class A

### Group CpG-A:

ODN2216:

(SEQ ID 71)

GGGGGACGATCGTCGGGGGG

### CpG Class B

### Group CpG-B:

### Natural Ligands:

ODN2006:

(SEQ ID 72)

TCGTCGTTTTGTCGTTTTGTCGTT

### Peptidic Ligands (Peptides):

| Name | SEQ ID | Sequence |
|---|---|---|
| 12-2 | 73 | ESWDKFLSHYLP |
| 7-6 | 74 | TDWSWFY |
| 7-7 | 75 | YPVYWPW |
| 7-12 | 76 | EWWFYWP |
| 7-13 | 77 | WFPIEWW |
| 7-37 | 78 | DQVDIGY |
| 7-38 | 79 | THQVYIS |
| 7-12/13 | 80 | WFPIEWWFYWP |
| 12-1 | 81 | DSWQAFLTKFVL |
| 12-3 | 82 | HDIQWFWQHWNS |
| 12-4 | 83 | WSWWDHTFNYML |
| 12-6 | 84 | TTQQTWNVRYPY |
| 12-8 | 85 | DHTMPWTRNAKN |
| 12-12 | 86 | SWDPYWPFPWFS |
| 12-14 | 87 | AIYYVPSPMFTV |
| 12-16 | 88 | ETTLLKMWLAQM |
| 12-18 | 89 | YPWLDVAVVSLY |
| 12-20 | 90 | VPGWHYLATLRA |
| 12-21 | 91 | FDPLGSRDIKGS |

As a CpG mimic in the molecule or molecule complex of the invention, such immunostimulatory peptides may be preferably used. Likewise functionally active variants thereof may be used, which are fragments, mutants, or hybrids, including combinations thereof.

Functionally active variants are specifically characterized in that they stimulate pDCs, thereby inducing an increased level of IL-6 and/or TNFalpha and/or IFNalpha, as compared to a negative control.

Functionally active variants of the immunostimulatory TLR9 binding peptides specifically

a) have at least 60% homology or sequence identity to any of the peptides of SEQ ID 73-91, preferably at least 70%, at least 80% or at least 90%;

b) are mutants of any of the peptides of SEQ ID 73-91, obtainable by modifying the parent amino acid sequence by insertion, deletion or substitution of one or more amino acids within the sequence or at either or both of the distal ends of the sequence, preferably less than 5, 4, 3, 2 or 1 point mutations; or

c) are fragments of any of the peptides of SEQ ID 73-91 comprising at least 50% of the parent sequence, or at least 60%, at least 70%, at least 80%, or at least 90%; or at least 5 amino acids, preferably at least 6, at least 7, at least 8, at least 9, at least 10 or at least 11 amino acids.

Specific functional variants comprise a motif selected from the group consisting of EWWFYWP (SEQ ID 101), EWW (SEQ ID 102), WFY (SEQ ID 103), YWP (SEQ ID 104), and QVxI, x being any amino acid (SEQ ID 105).

CpG Class C

Group CpG-C

ODNM362:

(SEQ ID 90)

TCGTCGTCGTTCGAACGACGTTGAT

7.3 Exemplary CD32 Binding Products with Coils

A coiled coil is a structural motif in polypeptides or peptides, in which two to seven alpha-helices are coiled together like the strands of a rope. Such alpha helical regions are likely to form coiled-coil structures and may be involved in oligomerization of the coil repeats as measured in a suitable coiled coil interaction binding assay.

Specifically a dimer of alpha-helices can be formed by contacting the two monomers, such that the dimer is formed through an interaction with the two alpha helix coiled coil domains.

### ScFV-Coil 1 (IV.3):

(SEQ ID 91)
EVQLQQSGPELKKPGETVKISCKASGYTFTNYGMNWVKQAPGKGLKWM
GWLNTYTGESIYPDDFKGRFAFSSETSASTAYLQINNLKNEDMATYFCAR
GDYGYDDPLDYWGQGTSVTVSSGGGGSGGGGSGGGGSDIVMTQAAPS
VPVTPGESVSISCRSSKSLLHTNGNTYLHWFLQRPGQSPQLLIYRMSV
LASGVPDRFSGSGSGTAFTLSISRVEAEDVGVFYCMQHLEYPLTFGAG
TKLELKGSISAWSHPQFEKGPEVSALEKEVSALEKEVSALEKEVSALEKE
VSALEK

Underlined: VH domain
Bold: HL domain
Normal type set. Flexible linker (maybe any linker)
In italics: pepE coil plus C′ StrepTag II sequence and “GP” linker may be any flexible linker (StrepTag II may be removed or replaced by HIS Tag or any other tag)

### ScFV-Coil 2 (AT10):

(SEQ ID 92)
EVKLEESGGGLVQPGGSMKLSCVASGFTFSYYWMNWVRQSPEKGLEWV
AEIRLKSNNYATHYAESVKGRFTISRDDSKNNVYLQMNNLRAEDTGIYYC
NRRDEYYAMDYWGQGTSVSVSSGGGGSGGGGSGGGGSDIVLTQSPGSL
AVSLGQRATISCRASESVDNFGISFMNWFQQKPGQPPRLLIYGASNQG
SGVPARFSGSGSGTDFSLNIHPVEEDDAAMYFCQQSKEVPWTFGGGT
KLEIKGSISAWSHPQFEKGPEVSALEKEVSALEKEVSALEKEVSALEKEV
SALEK

Underlined: VH domain
Bold: HL domain
Normal type set. Flexible linker (maybe any linker)
In italics: pepE coil plus at C′ StrepTag II sequence and “GP” linker may be any flexible linker (StrepTag II may be removed or replaced by HIS Tag or any other tag)

### Peptide-Coil:

(SEQ ID 93)
ADGAWAWVWLTETAVGAAKGPEVSALEKEVSALEKEVSALEKEVSALEKE
VSALEK

In italics: pepE coil plus “GP” linker may be any flexible linker

### IgG1 Fc Fragment-Coil:

(SEQ ID 94)
(PKSCDKTHTCPPCP)PELLGGPSVFLFPPKPKDTLMISRTPEVTCVVVD
VSHEDPEVKFNWYVDGVEVHNAKTKPREEQYNSTYRVVSVLTVLHQDWLN
GKEYKCKVSNKALPAPIEKTISKAKGQPREPQVYTLPPSRDELTKNQVS
LTCLVKGFYPSDIAVEWESNGQPENNYKTTPPVLDSDGSFFLYSKLTV
DKSRWQQGNVFSCSVMHEALHNHYTQKSLSLSPGKGPEVSALEKEVSA
LEKEVSALEKEVSALEKEVSALEK

Between ( ) is hinge region may be omitted
Underlined: CH2 domain
Bold CH3 domain
In italics: pepE coil plus “GP” linker may be any flexible linker

7.4. Exemplary TLR9 Binding Products with SH Group for Chemical Cross-Linking to the CD32 Binder

Group CpG-A:

ODN2216_SH:
(SEQ ID 69)
GGGGGACGATCGTCGGGGGG-SH

In bold, flexible linker with SH group for chemical cross-linking to ScFV-coil (Maybe any linker and chemically reactive group e.g. NH2 suited for chemical crosslinking)

Group CpG-B:

Natural Ligands:

ODN2006_SH:
(SEQ ID 70)
TCGTCGTTTTGTCGTTTTGTCGTT-SH

### Peptidic Ligands_SH:

| Name | SEQ ID | Sequence |
|---|---|---|
| 12-12_SH | 71 | SWDPYWPFPWFSGGGS-SH |
| 7-6_SH | 72 | TDWSWFYGGGS-SH |
| 7-7_SH | 73 | YPVYWPWGGGS-SH |
| 7-12_SH | 74 | EWWFYWPGGGS-SH |
| 7-13_SH | 75 | WFPIEWWGGGS-SH |
| 7-37_SH | 76 | DQVDIGYGGGS-SH |
| 7-38_SH | 77 | THQVYISGGGS-SH |
| 7-12/13_SH | 78 | WFPIEWWFYWPGGGS-SH |
| 12-1_SH | 79 | DSWQAFLTKFVLGGGS-SH |
| 12-2_SH | 80 | ESWDKFLSHYLPGGGS-SH |
| 12-3_SH | 81 | HDIQWFWQHWNSGGGS-SH |
| 12-4_SH | 82 | WSWWDHTFNYMLGGGS-SH |
| 12-6_SH | 83 | TTQQTWNVRYPYGGGS-SH |
| 12-8_SH | 84 | DHTMPWTRNAKNGGGS-SH |
| 12-14_SH | 85 | AIYYVPSPMFTVGGGS-SH |
| 12-16_SH | 86 | ETTLLKMWLAQMGGGS-SH |
| 12-18_SH | 87 | YPWLDVAVVSLYGGGS-SH |
| 12-20_SH | 88 | VPGWHYLATLRAGGGS-SH |
| 12-21_SH | 89 | FDPLGSRDIKGSGGGS-SH |

In bold, flexible linker with SH group for chemical crosslinking to ScFV-coil (Maybe any linker and chemically reactive group e.g. NH2 suited for chemical crosslinking)

Group CpG-C

ODNM362_SH:
(SEQ ID 90)
TCGTCGTCGTTCGAACGACGTTGAT-SH

In bold flexible linker with SH group for chemical crosslinking to ScFV-coil (Maybe any linker and chemically reactive group e.g. NH2 suited for chemical crosslinking)

7.5 Exemplary Warhead, i.e. a Structure Comprising a CD32 Binder and a TLR9 Binder

Any representative from the group of CD32 binders chemically linked by any method with any representative of the group of TLR9 binders, where preferably the TLR9 binders are coupled to available Lysines (K) in the CD32 binders e.g. Also mixtures of different TLR9 binders may be coupled e.g. CpG-B natural or peptidic binders.

### ScFV-Coil1 (IV.3)

(SEQ ID 91)
EVQLQQSGPELKKPGETVKISCKASGYTFTNYGMNWVKQAPGKGLKWMGWL
NTYTGESIYPDDFKGRFAFSSETSASTAYLQINNLKNEDMATYFCARGDYG
YDDPLDYWGQGTSVTVSSGGGGSGGGGSGGGGSDIVMTQAAPSVPVTPGES
VSISCRSSKSLLHTNGNTYLHWFLQRPGQSPQLLIYRMSVLASGVPDRFSG
SGSGTAFTLSISRVEAEDVGVFYCMQHLEYPLTFGAGTKLELKGSISAWSH
PQFEKGPEVSALE [symbol] EVSALE [symbol] EVSALE [symbol] EVSALE [symbol] EVSALE

[symbol]

Lysines in coil structure (Italic) are preferred

or

Peptide-Coil:

(SEQ ID 93)
ADGAWAWVWLTETAVGAAKGPEVSALE [symbol] EVSALE [symbol] EVSALE [symbol]
EVSALE [symbol] EVSALE

[symbol]

Lysines in coil structure (Italic) are preferred

7.6. Exemplary Immunogen, Herein Also Called Antigen

The term “immunogen” as used herein shall mean one or more antigens triggering an immune response in a subject. The term “antigen” as used herein shall in particular refer to any antigenic determinant, which can be possibly recognized by a binding site of an antibody or is able to bind to the peptide groove of HLA class I or class II molecules and as such may serve as stimulant for specific T cells. The target antigen is either recognized as a whole target molecule or as a fragment of such molecule, especially substructures, e.g. a polypeptide or carbohydrate structure of targets, generally referred to as “epitopes”, e.g. B-cell epitopes, T-cell epitope), which are immunologically relevant, i.e. are also recognizable by natural or monoclonal antibodies. Herein the use of T cell epitopes is preferred.

The term “epitope” as used herein according to the present invention shall in particular refer to a molecular structure which may completely make up a specific binding partner or be part of a specific binding partner to a binding site of modular antibody of the present invention. The term epitope may also refer to haptens. Chemically, an epitope may either be composed of a carbohydrate, a peptide, a fatty acid, an organic, biochemical or inorganic substance or derivatives thereof and any combinations thereof. If an epitope is a polypeptide, it will usually include at least 3 amino acids, preferably 8 to 50 amino acids, and more preferably between about 10-20 amino acids in the peptide. There is no critical upper limit to the length of the peptide, which could comprise nearly the full length of a polypeptide sequence of a protein. Epitopes can be either linear or conformational epitopes. A linear epitope is comprised of a single segment of a primary sequence of a polypeptide or carbohydrate chain. Linear epitopes can be contiguous or overlapping. Conformational epitopes are comprised of amino acids or carbohydrates brought together by folding of the polypeptide to form a tertiary structure and the amino acids are not necessarily adjacent to one another in the linear sequence. Specifically, epitopes are at least part of diagnostically relevant molecules, i.e. the absence or presence of an epitope in a sample is qualitatively or quantitatively correlated to either a disease or to the health status of a patient or to a process status in manufacturing or to environmental and food status. Epitopes may also be at least part of therapeutically relevant molecules, i.e. molecules which can be targeted by the specific binding domain which changes the course of the disease.

One or more epitopes of the same antigen or different antigens may be used according to the present invention, which can include antigens of all the self-antigens, pathogens, allergens or auto-antigens for which the regulation of the immune response is desired, e.g. against which induction of a substantial Th1-type response or Treg response (depending on the type of vaccine) in the host is desired.

In cancer disease an immune response to a self-antigen is desirable. The term “self-antigen” as used herein means any antigen, specifically polypeptide or peptide produced by a normal, healthy subject that does not elicit an immune response as such. These self-antigens may be produced at aberrant or high levels in certain disease states, including cancer disease, so called tumor associated antigens (TAAs). Self-antigens which are associated with auto-immune disease are herein called auto-antigens.

It is understood that the self-antigens can be naturally occurring, recombinantly or synthetically produced. It is also understood that the self-antigens need not be identical to the naturally produced antigen, but rather can include variations thereto having certain homology.

The choice of the self-antigen for use in cancer therapy depends on the type and stage of the cancer disease, and in particular on the expression pattern of a cancer cell such as derived from a tumor or metastases. Specific examples of selected tumor associated antigens possibly used in a vaccine according to the invention are Epithelial cell adhesion molecule (EpCAM), Lewis Y, alphafetoprotein (AFP) and carcinoembryonic antigen (CEA), HER2/Neu, VEGF, MUC-1, etc.

The choice of an auto-antigen for use in the therapy of auto-immune diseases depends on the type of the auto-immune disease. Specific examples of selected auto-immune disease associated antigens possibly used in a vaccine according to the invention are C1q, ADAMTS13, Desmogelin 3, keratin, gangliosides (e.g. GM1, GD1a, GQ1b), collagen type IV, IgM, cardiolipin, annexin A5, etc.

In some embodiments, the immunogen comprises one or more specific allergens. An “allergen” is an antigen which can initiate a state of hypersensitivity, or which can provoke an immediate hypersensitivity reaction in a subject already sensitized with the allergen. Allergens are commonly proteins or chemicals bound to proteins which have the property of being allergenic. However, allergens can also include organic or inorganic materials derived from a variety of synthetic or natural sources such as plant materials, metals, ingredients in cosmetics or detergents, latexes, or the like.

The choice of an allergen for use in the anti-allergy therapy depends on the type and severity of allergy. Specific examples of selected allergy associated antigens possibly used in a vaccine according to the invention are any allergen conventionally used as immunogen, specifically house dust mite allergens (e.g. Der p1, Der p2, Der p3, Der p5, Der p7/ - - - Der p23), cat dander, grass or tree pollen cockroach allergens, etc.

The choice of an antigen specifically inducing immune response against a pathogen for use in the prophylaxis or therapy of infectious diseases depends on the type of the pathogen, e.g. a microbial or viral infectious agent. Specific examples of selected pathogen derived antigens possibly used in a vaccine according to the invention are hepatitis B, hepatitis C, Cholera, HIV, Pertussis, Influenza, Typhoid, etc.

An exemplary antigen is an immunogen comprising one or more T cell epitopes of house dust mite allergens.

Specific antigens are selected from Immunogen 3 comprising the sequence of position 7-208 of SEQ ID 97, or Immunogen 5-12 comprising the sequence of position 1-364 of SEQ ID 98.

In a different embodiment, an exemplary antigen is a tumor associated antigen.

Immunogen 3 containing coil (Der P1 and Der P2 T cell epitopes based on human Class II expression): AA7-208 of SEQ ID 95.

(SEQ ID 95)
HHHHHHYYRYVAREQSCRRPNAQRFGISNYCQIYPPNVNKIREALAQTHS
AIAVDLRQMRTVTPIRMQGGCGSCWAFSGVAATESAYLQQYDIKYTWNVP
KIAPKSENVVVTVKVMGDDGVLACAIATHAKIRDDAFRHYDGRTIIQRDN
GYQPNYHAVNIVGYSNAQGVDYWIVRNSWDTNWHEIKKVLVPGCHGSEPC
IIHRGKPFGGGSGGGSGGKVSALKEKVSALKEKVSALKEKVSALKEKVSA
LKE

Underlined: HIS tag (may be removed)
In bold: a linker (can be any linker)
In italics: the pepK coil for interaction with warhead

Immunogen 5-12 containing coil (˜29 T cell epitopes of Der p1, Der p2, Der p3, Der p4, Der p7, Der p9, Der p10, Der p11, Der p14, Der p15, based on human Class II expression): AA1-364 of SEQ ID 96.

(SEQ ID 96)
GVLACAIATHAKIREQERLVKLETVKKSLEQEVRTLHVRIEEVEANALAG
GDLRQMRTVTPIRMQGGCGSCWEAHEQQIRIMTTKLKEAEARQQYDIKYT
WNVPKIAVNIVGYSNAQGVDYWIVRNSWDTNWYHNPHFIGNRSVITHLME
DLKGELDMRNIQVRGLKQMKRVGDANVKSEDDAFRHYDGRTIIQRDNGYQ
PNYLDEYWILTAAHCVDGQTVSKLIRSKVLGEKISYYRYVAREQSCRRPN
AQRFGISNYCVVVTVKVMGDDELHTYFNVNYTMHYYLNNGATRDILDEYW
ILTAAHCVAGQTASKLSTRYNSLKHSLFKYRPFKVNELNLEGEFGRELQH
KFRLMRNSQMEVEEGGGSHHHHHHGGGSGGKVSALKEKVSALKEKVSALK
EKVSALKEKVSALKE

Underlined: HIS tag (may be removed)
In bold: a linker (can be any linker)
In italics: the pepK coil for interaction with warhead

7.7 Exemplary Allergy Vaccine SG100 Against House Dust Mite (HDM)

The exemplary molecule complex is formed by chemical linkage, fusion and/or affinity binding, in particular by a coiled-coil structure.

Warhead (based on ScFV-coil1 IV.3+ODNM362) is mixed with Immunogen 5-12 in a ratio which indicates 90% of warhead is complexed with immunogen, no free immunogen (molar ratio of ˜1:1.5) and formulated on Alum.

7.8 Efficacy of SG100 in Rhesus Monkeys:

Methods:

5 healthy house dust mite (HDM) naïve rhesus monkeys were immunized 3× with SG100 (100 μg/shot) absorbed on Alum) on d0, d14 and d28. Blood samples were taken on d0 and d49 for T cell activation and antibody production.

### Antibody Immune Response:

Serum samples were tested in standard ELISA for IgG antibodies against warhead, immo 5-12 (Immo5), Der p1, Der p2, Der p5 and Der p7. The antigens were coated to maxisorb plates (1 μg/ml I PBS) overnight at 4° C., washed twice, blocked with PBS 1% BSA, washed twice incubated with the sera in a 1:1000 dilution for 1h at 4° C., washed twice and subsequently detected with anti-human-IgG-PO (cross reactive with rhesus monkey IgG).

### Cellular Immune Response:

Proliferation: PBMC (105/well) were cultured for 4 days (37° C./5% CO2/99% humidity) in 8 plex with medium, warhead (2 μg/ml), Immunogen (2 μg/ml), Der p1 (2 μg/ml), Der p2 (2 μg/ml), Der p5 (2 μg/ml) and Der p7 (2 μg/ml). As positive control Con A (Concanavaline A, Sigma) was used. Proliferation was measured by [3H]-thymidine (0.5 μCi/well) during the final 18 hrs of a 4 day culture. Cells were harvested for β-scintillation counting (Topcount NXT, Packerd, Ramsey, Minn., USA). Net counts per minutes were calculated by subtracting the counts of the medium control from the counts induced by the different antigens.

### Cytokine Production:

From each well of the 8plex stimulations of the proliferation experiment, 50 μl supernatant was taken after 24 h and pooled. The pooled supernatants were tested for the presence of IFN□ and IL-4 using commercially available ELISA kits from U-Cytech, Utrecht The Netherlands).

Results:

### Antibody Responses:

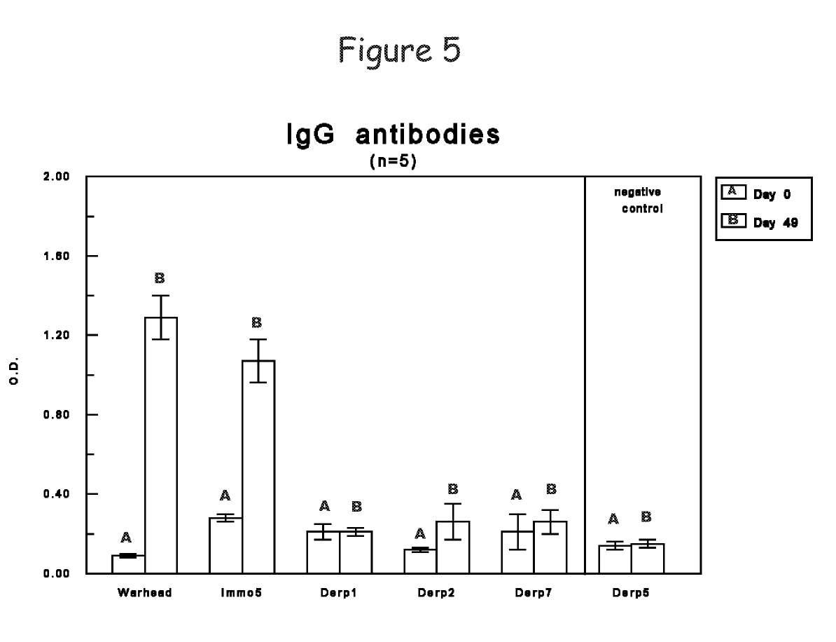

Strong IgG responses were measured against the warhead and the immunogen of SG100, but no antibodies were detected against Der p1, Der p2, Der p5 or Der p7 (FIG. 5), indicating that the animals were naive for the tested HDM allergens and that SG100 does not contain B cell epitopes, which cross-react with the tested HDM allergens.

### T Cell Response:

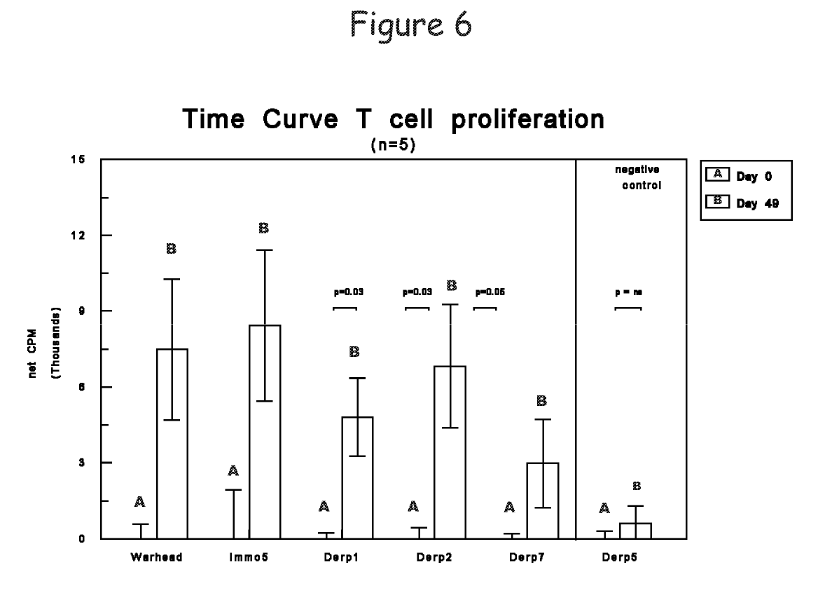

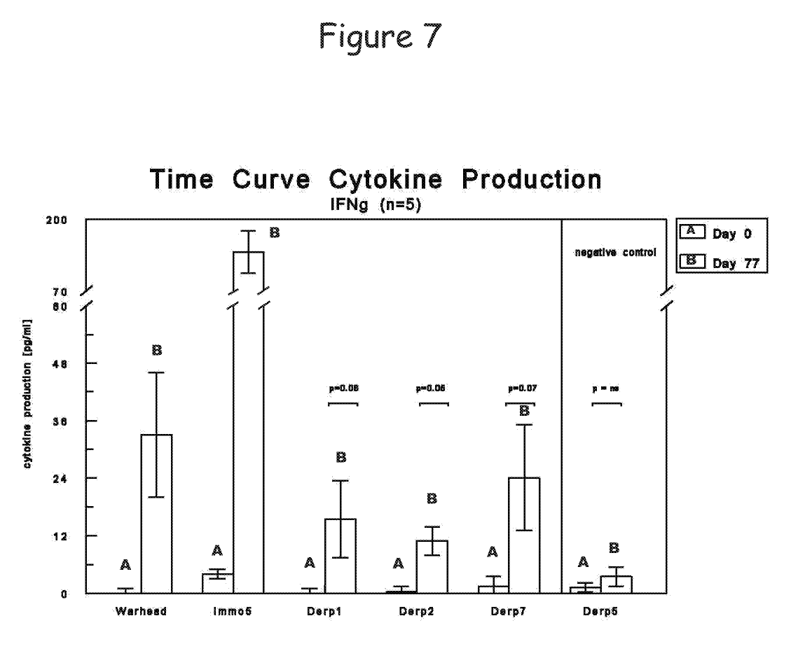

In FIG. 6 it can be seen that PBMCs of all treated animals showed strong proliferation when stimulated in vitro with warhead, immo5, Der p1, Der p2, Der p′7, but not against Der p5. Also IFN□ but no IL-4 was measured in supernatants from in vitro cultures with warhead, immo5, Der p1, Der p2, Der p7 but not against Der p5. (FIG. 7). IL-4 was seen after stimulation with Con A (data not shown).

Conclusion:

Immunization with SG100 induces a Th1 type memory response against the vaccine as indicated by the presence of IgG antibodies as well the induction of T cells which produce IFN□ but not IL-4 when stimulated by warhead or Immo5. As expected, no IgG (=B cell memory) against Der p1, Der p2, Der p5 or Der p7 was induced because the vaccine does not contain B ell epitopes from these allergens. However, Th1 type memory, was induced against the T cell epitopes of the house dust mite allergens which are present in the vaccine Der p1, Der p2, Der p7. No Th1 type memory is induced against Der p5, which is not included in the vaccine.

This confirms the concept of SG100.

7.7 Exemplary Vaccine, Warhead for Use in Oncology

### ScFV-Coil 1 (IV.3):

(SEQ ID 91)
EVQLQQSGPELKKPGETVKISCKASGYTFTNYGMNWVKQAPGKGLKWM
GWLNTYTGESIYPDDFKGRFAFSSETSASTAYLQINNLKNEDMATYFCAR
GDYGYDDPLDYWGQGTSVTVSSGGGGSGGGGSGGGGSDIVMTQAAPS
VPVTPGESVSISCRSSKSLLHTNGNTYLHWFLQRPGQSPQLLIYRMSV
LASGVPDRFSGSGSGTAFTLSISRVEAEDVGVFYCMQHLEYPLTFGAG
TKLELKGSISAWSHPQFEKGPEVSALEKEVSALEKEVSALEKEVSALEK
EVSALEK

Underlined: VH domain
Bold: HL domain
Normal type set. Flexible linker (maybe any linker)
In italics: pepE coil plus C′ StrepTag II sequence and “GP” linker may be any flexible linker (StrepTag II may be removed or replaced by HIS Tag or any other tag)
   Warhead with ODNM362:

EVQLQQSGPELKKPGETVKISCKASGYTFTNYGMNWVKQAPGKGLKW
MGWLNTYTGESIYPDDFKGRFAFSSETSASTAYLQINNLKNEDMATYFC
ARGDYGYDDPLDYWGQGTSVTVSSGGGGSGGGGSGGGGSDIVMTQAAPS
VPVTPGESVSISCRSSKSLLHTNGNTYLHWFLQRPGQSPQLLIYRMSVLA
SGVPDRFSGSGSGTAFTLSISRVEAEDVGVFYCMQHLEYPLTFGAGTKLE
LKGSISAWSHPQFEKGPEVSALE [symbol] EVSALE [symbol] EVSALE [symbol] EVSALE [symbol]
EVSALE

[symbol]

### ODNM362_SH

(SEQ ID 90)
TCGTCGTCGTTCGAACGACGTTGAT-SH

ODN-M362 may be coupled to any of the available lysines in ScFV-1-coil

Warhead (SG100):

ScFV-1-coil chemically linked with ODN-M362-SH. The preparation is a mix of ScFV-1-coil linked with 1 to 18 molecules ODN-M362 preferred is a mix with 1-6 molecules ODN-M362 coupled to ScFV-1-coil. All these mixes may be named warhead or ScFv-1-coil-M362.

Background:

Oncological targets for active immunotherapy are almost per definition autoantigens which are over expressed on tumor cells. These antigens are called tumor associated antigens (TAA) and the immune system is not able to respond against these antigens because they are recognized as self. A vaccine formulation that enables the immune system to generate a specific antibody and/or cellular immune response against an autoantigen is potentially suited for use as anti-tumor vaccination.

The warhead of SG100 enables an autoimmunresponse:

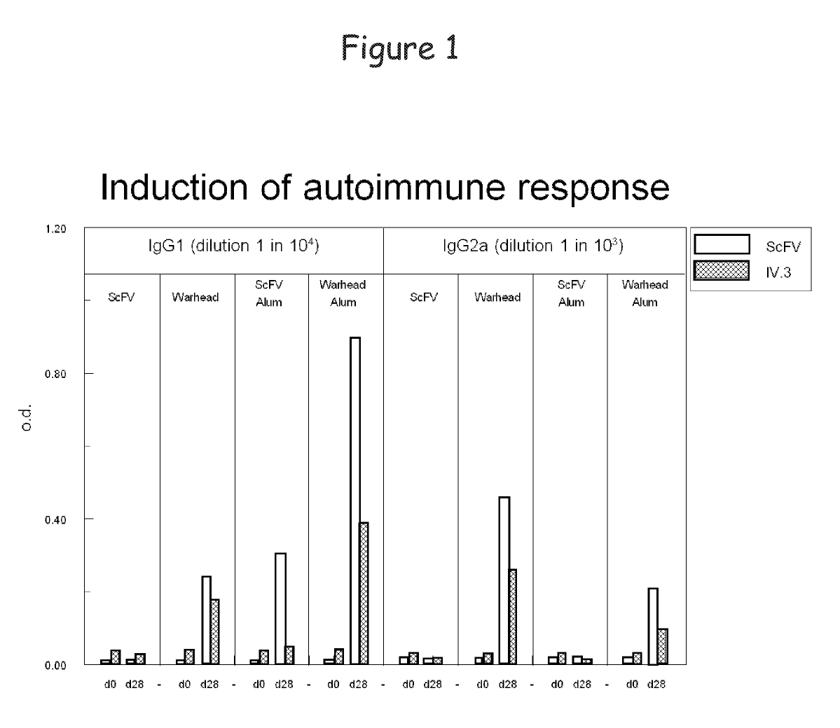

24 mice (6/group) were immunized s.c. 2× with 35 μg in 150 μl ScFV-1-coil or with warhead (ScFV-1-coil-M362) either formulated on Alum or diluted in PBS. Immunizations were done on d0, and d14, sera were taken on d0 (before immunization) and d28 and analyzed for IgG1 and IgG2a against ScFV-1-coil (indicated as ScFV) and mAb IV.3 by standard ELISA. See FIG. 1.

As can be seen in FIG. 1, immunization with warhead induced a strong IgG1 and IgG2 response to ScFV-1-coil as well as to mAb IV.3 on day 28. A positive response was seen independent of the presence of Alum. Immunization with ScFV-1-coil only induced an IgG1 response against ScFV-1-coil and only in the presence of Alum, no IgG2a response was induced. These data fit with the concept that CpG (M362) induces a Th1 type response (IgG2a) and Alum induces a TH2 type response (IgG1). The response against ScFV-1-coil indicates that this protein is immunogenic in the mouse, indeed both the StrepTagII (amino acid sequence “SAWSHPQFEK” (SEQ ID 97)) and the pepE (amino acid sequence “EVSALEKEVSALEKEVSALEKEVSALEKEVSALEK” SEQ ID 98) were target of the IgG responses (data not shown). However ScFV-1-coil also contains “mouse-self-sequences” because the ScFV contains the VH and VL domains of the mouse mAb IV.3. Therefore, an immune response against IV.3 indicates the presence of autoimmune antibodies. Indeed, only the warhead with or without Alum was able to induce this type of immune response. Hence, the presence of M362 on the ScFV-1-coil is able to break the tolerance against the autoantigens VH and VL domain of the parent antibody. By combining an autoantigen e.g. a TAA, through high affinity interaction with pepE of the warhead, the warhead will be able to induce the necessary autoimmune response against the TAA. The complex of the warhead (ScFV-1-coil-M362) with the TAA forms a potent vaccine for the treatment of cancer with over expression of the TAA in the vaccine. Such a vaccine may be formulated with any adjuvants, e.g. on Alum.

### Example 8: Comparison of the Stimulatory Capacity of the TLR9 Binder CPG on Human Plasmocytoid Dendritic Cells Administered in a Complex with an Anti-CD32 Antibody and in a Mixture

8.1 Material and Methods:

a) Plasmacytoid Dendritic (pDCs) Cells:

Buffy coats were obtained from healthy volunteers according to institutional guidelines and pDCs were purified by positive isolation using anti-BDCA-4-conjugated magnetic microbeads (Miltenyi Biotec) and cultured in X-VIVO-15 medium (Cambrex) supplemented with 2% of decomplemented human AB+ serum. pDCs purity was routinely up to 95%, as assessed by double staining BDCA-2/CD123 (Miltenyi Biotec) in a FACS.

b) Stimulation of pDCs:

Freshly isolated pDCs were incubated with biotinylated anti-CD32 (10 μg/ml, clone AT10, AbD Serotec) in PBA (PBS containing 5% BSA) on ice for 30 minutes and washed twice with PBA, followed by an incubation with 10 μg/ml streptavidin-Alexa647 in PBA on ice for 30 minutes and two times washing with PBA. Subsequently, pDCs were incubated on ice for 15 minutes with either PBA, 5 μg/ml ODN-CpG C (M362, Axorra) in PBA or 5 μg/ml ODN-CpG C-3′-biotin in PBA (M362, Biosearch Technologies). Unbound ODN-CpG C was washed away three time with PBA and pDCs were cultured overnight (37° C., 5% CO2) in X-VIVO-15 medium (Cambrex), supplemented with 2% of decomplemented human AB+ serum. Supernatants were collected from pDC cultures after overnight stimulation, and IFNα production was analyzed with murine monoclonal capture and HRP-conjugated anti-IFNα antibodies (BenderMed systems) using standard ELISA procedures.

8.2. Results:

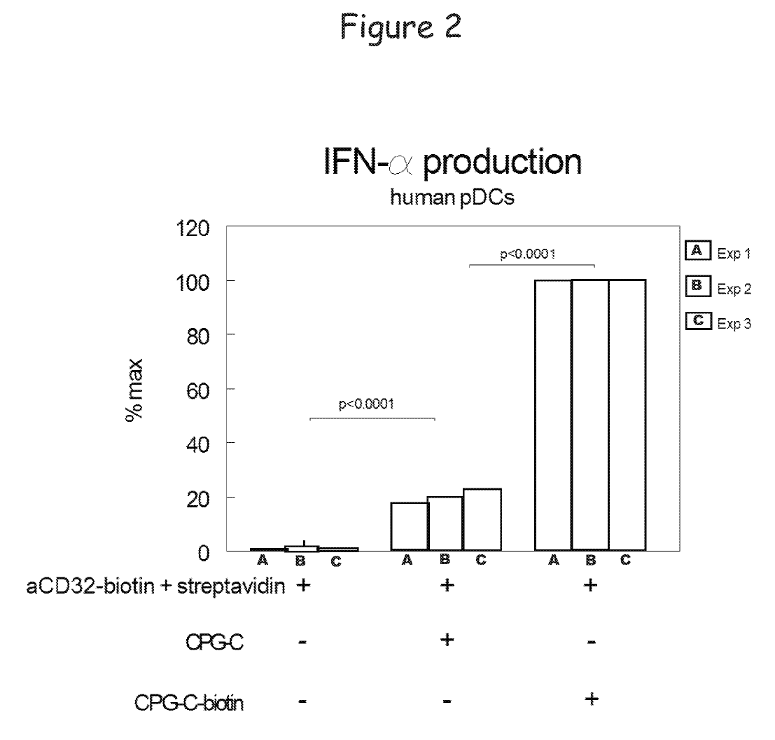

pDCs stimulated with 5 μg/ml CPG-C+aCD32-biotin/streptavidin-Alexa647 produced significantly more IFN-α, than pDCs stimulated with aCD32-biotin/streptavidin-Alexa647 alone. When aCD32 and CPG-C-biotin were complexed into one molecule, there was a statistically significant (p<0.001) positive synergistic effect on IFNα production compared to either aCD32-biotin/streptavidin-Alexa647 alone or aCD32-biotin/streptavidin-Alexa647+5 μg/ml CPG-C non-complexed. (FIG. 2). In a control experiment, there was no significant difference in IFNα production, when pDCs were stimulated with CPG-C versus CPG-biotin or CPG-C versus CPG-C+aCD32-biotin/streptavidin-Alexa647 (data not shown).

FIG. 2: IFNα Production of pDCS after Stimulation with CPG-C.

Maximum IFNα production in each experiment was seen when CPG-C-biotin was complexed with aCD32biotin through streptavidin. This was set at 100% and the ratio was calculated for aCD32-biotin with or without CPG-C (non complexed). Statistical analyses was done with the paired student t-test; p<0.05 were considered significant.

### Example 9: Effect of a Molecule Complex Comprising Different Formats of Anti-CD32 and TLR9 Binding Moieties on the Immune Response as Determined on Plasmacytoid Dendritic (pDCs) Cells

Stimulation of pDCs with CpG Targeted to CD32, Employing an Anti-CD32 Single Chain Antibody

9.1 Material and Methods:

a) Plasmacytoid Dendritic (pDCs) Cells:

Buffy coats were obtained from healthy volunteers according to institutional guidelines and pDCs were purified by positive isolation using anti-BDCA-4-conjugated magnetic microbeads (Miltenyi Biotec) and cultured in X-VIVO-15 medium (Cambrex) supplemented with 2% of decomplemented human AB+ serum. pDCs purity was routinely up to 95%, as assessed by double staining BDCA-2/CD123 (Miltenyi Biotec) in a FACS.

b) Stimulation of pDCs:

Freshly isolated pDCs were incubated with biotinylated anti-CD32 (10 μg/ml, clone AT10, AbD Serotec; ScFV from IV.3) in PBA (PBS containing 5% BSA) on ice for 30 minutes and washed twice with PBA, followed by an incubation with 10 μg/ml streptavidin-Alexa647 in PBA on ice for 30 minutes and two times washing with PBA. Subsequently, pDCs were incubated on ice for 15 minutes with either PBA, 5 μg/ml ODN-CpG C (M362, Axorra) in PBA or 5 μg/ml ODN-CpG C-3′-biotin in PBA (M362, Biosearch Technologies). Unbound ODN-CpG C was washed away three time with PBA and pDCs were cultured overnight (37° C., 5% CO2) in X-VIVO-15 medium (Cambrex), supplemented with 2% of decomplemented human AB+ serum. Supernatants were collected from pDC cultures after overnight stimulation, and IFNα production was analyzed with murine monoclonal capture and HRP-conjugated anti-IFNα antibodies (BenderMed systems) using standard ELISA procedures.

9.2. Results:

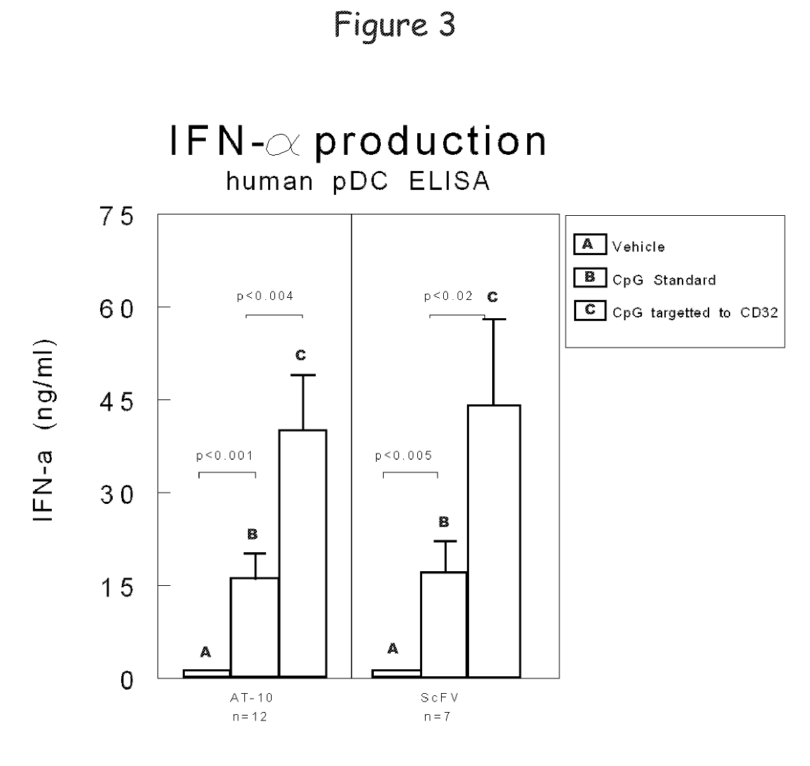

IFNα production of pDCS after stimulation with CPG-C: Maximum IFNα production in each experiment was seen when CPG-C-biot was complexed with aCD32biot through streptavidin. Stimulation with CpG without targeting (CpG standard) was significantly less potent than CpG targeted to CD32 (p<0.04 resp. p<0.02). There is no difference when the aCD32 antibody AT10 (specific for CD32a and CD32b) was used or whether an unrelated single chain antibody (specific for CD32a only) was used. This confirms that the enhancement is independent of the epitope that is recognized on CD32 and independent of the type of binder that is used. Statistical analyses was done with the paired student t-test; p<0.05 were considered significant (see FIG. 3).

### Example 10: Effect of a Molecule Complex Comprising Different Formats of Anti-CD32 and TLR9 Binding Moieties on the Immune Response as Determined on Plasmacytoid Dendritic (pDCs) Cells

Stimulation of pDCs with CpG Targeted to CD32, Employing an aCD32 Peptide, Read Out IL-6 and TNFα Production

10.1 Material and Methods:

a) PBMC Cells:

Buffy coats were obtained from the Red Cross Austria. Cells were separated on ficoll hypack.

b) Stimulation of pDCs:

Freshly isolated PBMC's were incubated with 200 μg/ml aCD32a peptide published by Berntzen et al (J. Biol. Chem. (2009) 284:1126-1135; sequence ADGAWAWVWLTETAVGAAK-biotin) in PBA (PBS containing 5% BSA) on ice for 30 minutes and washed twice with PBA, followed by an incubation with 10 μg/ml streptavidin in PBA on ice for 30 minutes and two times washing with PBA. Subsequently, PBMC's were incubated on ice for 15 minutes with either PBA, 5 μg/ml ODN-CpG C (M362, Axorra) in PBA or 5 μg/ml ODN-CpG C-3′-biotin in PBA (M362, Girindus). Unbound ODN-CpG C was washed away three time with PBA and PBMCs were cultured overnight (37° C., 5% CO2) in X-VIVO-15 medium (Cambrex), supplemented with 2% of decomplemented human AB+ serum. Supernatants were collected from PBMC cultures after overnight stimulation, and IFNα production was analyzed with murine monoclonal capture and HRP-conjugated anti-IL-6 or TNFα antibodies (BenderMed systems) using standard ELISA procedures.

10.2. Results:

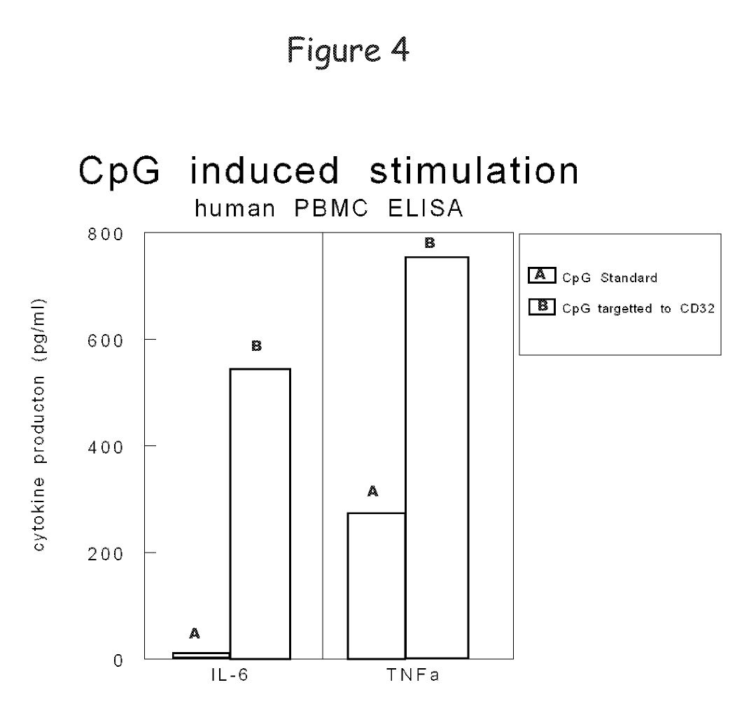

IL-6 production of pDCS in PBMC after stimulation with CPG-C: Maximum IL-6 production was seen when CPG-C-biot was complexed with aCD32biot through streptavidin. Stimulation with CpG without targeting (CpG standard) was ˜75 times less potent than CpG targeted to CD32. Whereas for TNFα the induction was ˜3 times stronger when CpG was targeted to CD32. This confirms that the enhancement is independent of the epitope that is recognized on CD32 and independent of the type of binder that is used. Statistical analyses was done with the paired student t-test; p<0.05 were considered significant (see FIG. 4).

Although the present invention has been described in considerable detail with reference to certain preferred embodiments, other embodiments are possible. The steps disclosed for the present methods, for example, are not intended to be limiting nor are they intended to indicate that each step is necessarily essential to the method, but instead are exemplary steps only. Therefore, the scope of the appended claims should not be limited to the description of preferred embodiments contained in this disclosure.

Recitation of value ranges herein is merely intended to serve as a shorthand method for referring individually to each separate value falling within the range. Unless otherwise indicated herein, each individual value is incorporated into the specification as if it were individually recited herein. All references cited herein are incorporated by reference in their entirety.

### REFERENCES

1. Mudde. G. C, I. G. Reischl, N. Corvaïa, A. Hren, and E.-M. Pollabauer. 1996. Antigen presentation in allergic sensitization. Immunol. Cell Biol. 74:167-173.
2. Bheekha Escura. R., E. Wasserbauer, F. Hammerschmid, A. Pearce, P. Kidd, and G. C. Mudde. 1995. Regulation and targeting of T-cell immune responses by IgE and IgG antibodies. Immunology 86:343-350.
3. Pene. J., F. Rousset, F. Briere, I. Chretien, J.-Y. Bonnefoy, H. Spits, T. Yokota, K.-I. Arai, J. Banchereau, and J. E. De Vries. 1988. IgE production by normal human lymphocytes is induced by interleukin 4 and suppressed by interferons gamma and alpha and prostaglandin E2. Proc. Natl. Acad. ScL USA 85:6880-6884.
4. Ebner. C. 1999. Immunological mechanisms operative in allergen-specific immunotherapy. IntArch Allergy Immunol 119:1-5.
5. Ferreira. F., C. Ebner, B. Kramer, G. Casari, P. Briza, A. J. Kungl, R. Grimm, B. Jahn-Schmid, H. Breiteneder, D. Kraft, M. Breitenbach, H. J. Rheinberger, and O. Scheiner. 1998. Modulation of IgE reactivity of allergens by site-directed mutagenesis: potential use of hypoallergenic variants for immunotherapy. FASEB Journal 12:231-242.
6. Rissoan. M. C, V. Soumelis, N. Kadowaki, G. Grouard, F. Briere, R. D. Malefyt, and Y. J. Liu. 1999. Reciprocal control of T helper cell and dendritic cell differentiation. Science 283:1183-1186.
7. Kapsenberg. M. L, C. M. Hilkens, E. A. Wierenga, and P. Kalinski. 1999. The paradigm of type 1 and type 2 antigen-presenting cells. Implications for atopic allergy. Clin. Exp. Allergy 29:33-36.
8. Charbonnier. A. S., H. Hammad, P. Gosset, G. A. Stewart, S. Alkan, A. B. Tonnel, and J. Pestel. 2003. Der p1-pulsed myeloid and plasmacytoid dendritic cells from house dust mite-sensitized allergic patients dysregulate the T cell response. J. Leukocyte Biol. 73:91-99.
9. Rothenfusser. S., E. Tuma, S. Endres, and G. Hartmann. 2002. Plasmacytoid dendritic cells: The key to CpG. Hum. Immunol. 63:1111-1119.
10. Latz. E., A. Schoenemeyer, A. Visintin, K. A. Fitzgerald, B. G. Monks, C. F. Knetter, E. Lien, N. J. Nilsen, T. Espevik, and D. T. Golenbock. 2004. TLR9 signals after translocating from the ER to CpG DNA in the lysosome. Nat. Immunol. 5:190-198.
11. Leifer. C A, M. N. Kennedy, A. Mazzoni, C. W. Lee, M. J. Kruhlak, and D. A. Segal. 2004. TLR9 Is localized in the endoplasmic reticulum prior to stimulation. J. Immunol. 173:1179-1183.
12. Wang. W. W., D. Das, X. L. Tang, W. Budzynski, and M. R. Suresh. 2005. Antigen targeting to dendritic cells with bispecific antibodies. J. Immunol. Methods.
13. van Schaijk. F. G., E. Oosterwijk, A. C. Soede, M. Broekema, C. Frielink, W. J. McBride, D. M. Goldenberg, F. H. Corstens, and O. C. Boerman. 2005. Pretargeting of carcinoembryonic antigen-expressing tumors with a biologically produced bispecific anticarcinoembryonic antigen x anti-indium-labeled diethylenetriaminepentaacetic acid antibody. CHn. Cancer Res. 11:7130s-7136s.
14. Le. G. F., U. Reusch, G. Moldenhauer, M. \ddot{υ}ttle, and S. M. Kipriyanov. 2004. Immunosuppressive properties of anti-CD3 single-chain Fv and diabody. J. Immunol. Methods 285:111-127.
15. Schuster, M., P. Umana, C. Ferrara, P. Brunker, C. Gerdes, G. Waxenecker, S. Wiederkum, C. Schwager, H. Loibner, G. Himmler, and G. C. Mudde. 2005. Improved effector functions of a therapeutic monoclonal Lewis Y-specific antibody by glycoform engineering. Cancer Res 65:7934-7941.
16. Orlandi. R., D. H. Gussow, P. T. Jones, and G. Winter. 1989. Cloning immunoglobulin variable domains for expression by the polymerase chain reaction. Proc. Natl. Acad. Sci. U. S. A 86:3833-3837.
17. Barton. G. M., J. C. Kagan, and R. Medzhitov. 2005. Intracellular localization of Toll-like receptor 9 prevents recognition of self DNA but facilitates access to viral DNA. Nat. Immunol.
18. Chen. W. L, J. L. Wang, H. Z. An, J. Zhou, L. H. Zhang, and X. T. Cao. 2005. Heat shock up-regulates TLR9 expression in human B cells through activation of ERK and NF-kappaB signal pathways. Immunol. Lett. 98:153-159.
19. Eaton-Bassiri. A., S. B. Dillon, M. Cunningham, M. A. Rycyzyn, J. Mills, R. T. Sarisky, and M. L. Mbow. 2004. Toll-like receptor 9 can be expressed at the cell surface of distinct populations of tonsils and human peripheral blood mononuclear cells. Infect. Immun. 72:7202-7211.
20. Leroy. B. P., J.-M. Lachapelle, M. Jacquemin, and J.-M. Saint-Remy. 1992. Treatment of atopic dermatitis by allergen-antibody complexes: Long-term clinical results and evolution of IgE antibodies. Dermatologica 184:271-274.
21. Chua. K. Y., W. K. Greene, P. Kehal, and W. R. Thomas. 1991. IgE binding studies with large peptides expressed from Der p Il cDNA constructs. CHn. Exp. Allergy. 21:161-166.
22. Baselmans. P. J., E. Pollabauer, F. C. Van Reijsen, H. C. Heystek, A. Hren, P. Stumptner, M. G. Tilanus, W. C. Vooijs, and G. C. Mudde. 2000. IgE production after antigen-specific and cognate activation of HLA-DPw4-restricted T-cell clones, by 78% of randomly selected B-cell donors. Hum. Immunol. 61:789-798.
23. Beck. H., G. Schwarz, C. J. Schroter, M. Deeg, D. Baier, S. Stevanovic, E. Weber, C. Driessen, and H. Kalbacher. 2001. Cathepsin S and an asparagine-specific endoprotease dominate the proteolytic processing of human myelin basic protein in vitro. Eur J Immunol 31:3726-3736.
24. Higgins. J. A., C. J. Thorpe, J. D. Hayball, R. E. OHehir, and J. R. Lamb. 1994. Overlapping T-cell epitopes in the group I allergen of Dermatophagoides species restricted by HLA-DP and HLA-DR class Il molecules. J. Allergy CHn. Immunol. 93:891-899.
25. Bian. H., J. F. Reidhaar-OIson, and J. Hammer. 2003. The use of bioinformatics for identifying class ll-restricted T-cell epitopes. Methods 29:299-309.
26. Sturniolo. T., E. Bono, J. Ding, L. Raddhzzani, O. Tuereci, U. Sahin, M. Braxenthaler, F. Gallazzi, M. P. Protti, F. Sinigaglia, and J. Hammer. 1999. Generation of tissue-specific and promiscuous HLA ligand databases using DNA microarrays and virtual HLA class Il matrices. Nat Biotechnol. 17:555-561.

1. A composition comprising a molecule or molecule complex, wherein the molecule or molecule complex comprises a TLR9 binding part capable of binding to TLR9, a CD32 binding part capable of binding to CD32, and at least one antigen,
wherein the CD32 binding part is selected from the group consisting of CD32-binding protein domains, CD32-binding peptides, and CD32-binding nucleic acids, and
wherein the TLR9 binding part is a CpG oligodeoxynucleotide.

2. The composition of claim 1, wherein the antigen is non-covalently linked to the molecule or molecule complex.

3. The composition of claim 1, wherein the antigen is non-covalently linked to the TLR9 binding part or the CD32 binding part.

4. The composition of claim 3, wherein the antigen is linked to the TLR9 binding part or the CD32 binding part via ligand interaction.

5. The composition of claim 1, wherein the antigen is selected from the group consisting of an allergen associated with atopic dermatitis, an allergen associated with allergic asthma, an allergen associated with allergic rhinitis or an allergen associated with allergic conjunctivitis.

6. The composition of claim 1, wherein the antigen is isolated from a source selected from the group consisting of complete antigens, denatured antigens, and antigens modified to prevent binding to IgE.

7. The composition of claim 1, wherein at least part of the TLR9 binding part or the CD32 binding part is derived from a mammal selected from the group consisting of a human, a mouse, and a camel.

8. The composition of claim 1, wherein the molecule or molecule complex comprises at least one engineered binding scaffold.

9. The composition of claim 8, wherein the binding scaffold is selected from the group consisting of fibronectin III, lipocalins, Protein A, α-amylase inhibitor, Ankyrin Repeat Proteins, a C2 domain, an A-domain, an EGFR like domain, a dab, a chi-bAb, and CTLA 4.

10. The composition of claim 1, wherein the composition comprises at least one pharmaceutically acceptable carrier or diluent.

11. The composition of claim 1, wherein the TLR9 binding part is selected from the group consisting of CpG-A, CpG-B and CpG-C.

12-13. (canceled)

14. The composition of claim 1, wherein the CD32-binding peptide is a CD32a peptide comprising the sequence of SEQ ID 66.

15. The composition of claim 1, wherein the antigen comprises a house dust mite allergen.

16. The composition of claim 15, wherein the antigen is Immunogen 3 comprising the sequence of position 7-208 of SEQ ID 97, or Immunogen 5-12 comprising the sequence of position 1-364 of SEQ ID 98.

17. The composition of claim 1, wherein the antigen is a tumor associated antigen.

18. The composition of claim 1, wherein the CD32 binding part is a CD32-binding protein domain selected from the group consisting of an EGF-like domain, a Kringle-domain, a fibronectin type I domain, a fibronectin type Il domain, a fibronectin type III domain, a PAN domain, a GIa domain, a SRCR domain, a Kunitz/Bovine pancreatic trypsin Inhibitor domain, a Kazal-type serine protease inhibitor domain, a Trefoil (P-type) domain, a von Willebrand factor type C domain, an Anaphylatoxin-like domain, a CUB domain, a thyroglobulin type I repeat, a LDL-receptor class A domain, a Sushi domain, a Link domain, a Thrombospondin type I domain, an immunoglobulin domain, an Immunoglobulin-like domain, a C-type lectin domain, a MAM domain, a von Willebrand factor type A domain, an A-domain, a Somatomedin B domain, a WAP-type four disulfide core domain, an F5/8 type C domain, a Hemopexin domain, an SH2 domain, an SH3 domain, a Laminin-type EGF-like domain, a CTLA-4 domain, and a C2 domain.


SEQUENCE LISTING (105 sequences)

SEQ ID NO: 1 - protein, 14 aa, human
CPRHFPQLHPDTFS

SEQ ID NO: 2 - protein, 16 aa, human
LTHLSLKYNNLTVVPR

SEQ ID NO: 3 - protein, 25 aa, human
ANLTALRVLDVGGNCRRCDHAPNPC

SEQ ID NO: 4 - protein, 43 aa, Dermatophagoides pteronyssinus
QSCRRPNAQRFGISNYCQIYPPNANKIREALAQPQRYCRHYWT

SEQ ID NO: 5 - protein, 31 aa, Dermatophagoides pteronyssinus
QSCRRPNAQRFGISNYCQIYPPNANKIREAL

SEQ ID NO: 6 - protein, 13 aa, Dermatophagoides pteronyssinus
NAQRFGISNYCQI

SEQ ID NO: 7 - protein, 25 aa, Dermatophagoides pteronyssinus
RTVTPIRMQGGCGSCWAFSGVAATE

SEQ ID NO: 8 - protein, 25 aa, Dermatophagoides pteronyssinus
YDGRTIIQRDNGYQPNYHAVNIVGY

SEQ ID NO: 9 - protein, 19 aa, Dermatophagoides pteronyssinus
PCIIHRGKPFQLEAVFEAN

SEQ ID NO: 10 - protein, 19 aa, Dermatophagoides pteronyssinus
KYTWNVPKIAPKSENVVVT

SEQ ID NO: 11 - protein, 22 aa, Dermatophagoides pteronyssinus
ENVVVTVKVMGDDGVLACAIAT

SEQ ID NO: 12 - protein, 22 aa, Dermatophagoides pteronyssinus
QSCRRPNAQRFGISNYCQIYPP

SEQ ID NO: 13 - protein, 15 aa, Dermatophagoides pteronyssinus
CQIYPPNANKIREAL

SEQ ID NO: 14 - protein, 17 aa, Dermatophagoides pteronyssinus
IREALAQPQRYCRHYWT

SEQ ID NO: 15 - DNA, 36 bp, human
aaacgggcagatgctgcaccaactgtatccatcttc

SEQ ID NO: 16 - protein, 164 aa, human
CQIYPPNANKIREALQSCRRPNAQRFGISNYCQIYPPYDGRTIIQRDNGYQPNYHAVNIVGYENVVVTVKVMGDDGVLACAIATKYTWNVPKIAPKSENVVVTIREALAQPQRYCRHYWTPCIIHRGKPFQLEAVFEANRTVTPIRMQGGCGSCWAFSGVAATE

SEQ ID NO: 17 - DNA, 28 bp, artificial sequence
  oligonucleotide for generation of synthetic gene coding for the allergen epitope
tccggatgccaaatttacccgccaaacg

SEQ ID NO: 18 - DNA, 40 bp, artificial sequence
  oligonculeotide for generation of synthetic gene coding for allergen epitope
agcctctctgatcttgttcgcgtttggcgggtaaatttgg

SEQ ID NO: 19 - DNA, 40 bp, artificial sequence
  oligonucleotide for generation of synthetic gene coding for allergen epitope
cgaacaagatcagagaggctttgcaatcttgcaggaggcc

SEQ ID NO: 20 - DNA, 40 bp, artificial sequence
  oligonucleotide for generation of synthetic gene coding for allergen epitope
tatgccgaatctctgcgcattgggcctcctgcaagattgc

SEQ ID NO: 21 - DNA, 40 bp, artificial sequence
  oligonucleotide for generation of synthetic gene coding for allergen epitope
gcgcagagattcggcatatccaactactgccagatctacc

SEQ ID NO: 22 - DNA, 40 bp, artificial sequence
  oligonucleotide for generation of synthetic gene coding for allergen epitope
gtacgcccatcgtatggggggtagatctggcagtagttgg

SEQ ID NO: 23 - DNA, 40 bp, artificial sequence
  oligonucleotide for generation of synthetic gene coding for allergen epitope
cccatacgatgggcgtacaatcatacagcgtgataacggc

SEQ ID NO: 24 - DNA, 40 bp, artificial sequence
  oligonucleotide for generation of synthetic gene coding for allergen epitope
gcgtggtagttaggctgatagccgttatcacgctgtatga

SEQ ID NO: 25 - DNA, 40 bp, artificial sequence
  oligonucleotide for generation of synthetic gene coding for allergen epitope
tatcagcctaactaccacgccgtgaacatcgtcggctacg

SEQ ID NO: 26 - DNA, 40 bp, artificial sequence
  oligonucleotide for generation of synthetic gene coding for allergen epitope
tcacagtaaccacgacattctcgtagccgacgatgttcac

SEQ ID NO: 27 - DNA, 40 bp, artificial sequence
  oligonucleotide for generation of synthetic gene coding for allergen epitope
agaatgtcgtggttactgtgaaggtaatgggcgatgacgg

SEQ ID NO: 28 - DNA, 40 bp, artificial sequence
  oligonucleotide for generation of synthetic gene coding for allergen epitope
agctatggcgcaagctagaaccccgtcatcgcccattacc

SEQ ID NO: 29 - DNA, 40 bp, artificial sequence
  oligonucleotide for generation of synthetic gene coding for allergen epitope
tctagcttgcgccatagctaccaagtacacttggaacgta

SEQ ID NO: 30 - DNA, 40 bp, artificial sequence
  oligonucleotide for generation of synthetic gene coding for allergen epitope
ttttcggcgcaattttgggtacgttccaagtgtacttggt

SEQ ID NO: 31 - DNA, 40 bp, artificial sequence
  oligonucleotide for generation of synthetic gene coding for allergen epitope
cccaaaattgcgccgaaaagtgaaaacgtcgtagtgacca

SEQ ID NO: 32 - DNA, 40 bp, artificial sequence
  oligonucleotide for generation of synthetic gene coding for allergen epitope
tgagccaatgcctcccttatggtcactacgacgttttcac

SEQ ID NO: 33 - DNA, 40 bp, artificial sequence
  oligonucleotide for generation of synthetic gene coding for allergen epitope
agggaggcattggctcaacctcaaagatactgcagacact

SEQ ID NO: 34 - DNA, 40 bp, artificial sequence
  oligonucleotide for generation of synthetic gene coding for allergen epitope
ttatgcagggcgtccagtagtgtctgcagtatctttgagg

SEQ ID NO: 35 - DNA, 40 bp, artificial sequence
  oligonucleotide for generation of synthetic gene coding for allergen epitope
actggacgccctgcataatccaccgtggtaaaccctttca

SEQ ID NO: 36 - DNA, 40 bp, artificial sequence
  oligonucleotide for generation of synthetic gene coding for allergen epitope
cttcgaacactgcctcaagttgaaagggtttaccacggtg

SEQ ID NO: 37 - DNA, 40 bp, artificial sequence
  oligonucleotide for generation of synthetic gene coding for allergen epitope
acttgaggcagtgttcgaagctaacaggacggtaacgcca

SEQ ID NO: 38 - DNA, 40 bp, artificial sequence
  oligonucleotide for generation of synthetic gene coding for allergen epitope
ccgcacccaccttgcatacgaattggcgttaccgtcctgt

SEQ ID NO: 39 - DNA, 40 bp, artificial sequence
  oligonucleotide for generation of synthetic gene coding for allergen epitope
tgcaaggtgggtgcgggtcttgttgggctttttctggtgt

SEQ ID NO: 40 - DNA, 42 bp, artificial sequence
  oligonucleotide for generation of synthetic gene coding for allergen epitope
actagtttattcagtagcagccacaccagaaaaagcccaaca

SEQ ID NO: 41 - protein, 6 aa, human
KRAPRE

SEQ ID NO: 42 - DNA, 18 bp, artificial sequence
  silent mutation to introduce unique XhoI site
aaacgggctcctcgagaa

SEQ ID NO: 43 - protein, 219 aa, artificial sequence
  complete sequence of a VL-CH3 fusion protein
DIVMTQAAPSVPVTPGESVSISCRSSKSLLHTNGNTYLHWFLQRPGQSPQLLIYRMSVLASGVPDRFSGSGSGTAFTLSISRVEAEDVGVFYCMQHLEYPLTFGAGTKLELKRAPREPQVYTLPPSRDELGIAQVSLTCLVKGFYPSDIAVEWESNGQPENNYKTTPPVLDSDGSFFLYSKLTVLGRRWTLGNVFSCSVMHEALHNHYTQKSLSLSPGK

SEQ ID NO: 44 - DNA, 681 bp, artificial sequence
  nucleic acid sequence of VL-CH3 fusion protein
gaattcgacattgtgatgacccaggctgcaccctctgtacctgtcactcctggagagtcagtatccatctcctgcaggtctagtaagagtctcctgcatactaatggcaacacttacttgcattggttcctacagaggccaggccagtctcctcagctcctgatatatcggatgtccgtccttgcctcaggagtcccagacaggttcagtggcagtgggtcaggaactgctttcacactgagcatcagtagagtggaggctgaggatgtgggtgttttttactgtatgcaacatctagaatatccgctcacgttcggtgctgggaccaagctggaactgaaacgggctcctcgagaaccacaggtgtacaccctgcccccatcccgggacgagctcggcatcgcgcaagtcagcctgacctgcctggtcaaaggcttctatcccagcgacatcgccgtggagtgggagagcaacgggcagccggagaacaactacaagaccacgcctcccgtgctggactccgacggctctttcttcctctacagcaagcttaccgtgttgggccgcaggtggaccctggggaacgtcttctcatgctccgtgatgcatgaggctctgcacaaccactacacacagaagagcctctccctgtctccgggtaaatgagggcgcgccggtacc

SEQ ID NO: 45 - protein, 6 aa, artificial sequence
  part of heavy chain of CH3 domain
AKTREP

SEQ ID NO: 46 - DNA, 18 bp, artificial sequence
  VH-CH3 with silent mutation to introduce XhoI site
gccaaaactcgagaacca

SEQ ID NO: 47 - protein, 250 aa, artificial sequence
  complete sequence of VH-CH3 fusion protein
EVQLQQSGPELKKPGETVKISCKASGYTFTNYGMNWVKQAPGKGLKWMGWLNTYTGESIYPDDFKGRFAFSSETSASTAYLQINNLKNEDMATYFCARGDYGYDDPLDYWGQGTSVTVSSAKTREPQVYTLPPSRDELGIAQVSLTCLVKGFYPSDIAVEWESNGQPENNYKTTPPVLDSDGSFFLYSKLTVLGRRWTLGNVFSCSVMHEALHNHYTQKSLSLSPGKSLEQKLISEEDLNSAVDHHHHHH

SEQ ID NO: 48 - DNA, 759 bp, artificial sequence
  nucleic acid sequence of VH-CH3 complete fusion protein
gaattcgaggttcagcttcagcagtctggacctgagctgaagaagcctggagagacagtcaagatctcctgcaaggcttctgggtataccttcacaaactatggaatgaactgggtgaagcaggctccaggaaagggtttaaagtggatgggctggttaaacacctacactggagagtcaatatatcctgatgacttcaagggacggtttgccttctcttcggaaacctctgccagcactgcctatttgcagatcaacaacctcaaaaatgaggacatggctacatatttctgtgcaagaggggactatggttacgacgaccctttggactactggggtcaaggaacctcagtcaccgtctcctcagccaaaactcgagaaccacaggtgtacaccctgcccccatcccgggacgagctcggcatcgcgcaagtcagcctgacctgcctggtcaaaggcttctatcccagcgacatcgccgtggagtgggagagcaacgggcagccggagaacaactacaagaccacgcctcccgtgctggactccgacggctctttcttcctctacagcaagcttaccgtgttgggccgcaggtggaccctggggaacgtcttctcatgctccgtgatgcatgaggctctgcacaaccactacacacagaagagcctctccctgtctccgggtaaatctctagaacaaaaactcatctcagaagaggatctgaatagcgccgtcgaccatcatcatcatcatcattga

SEQ ID NO: 49 - DNA, 30 bp, artificial sequence
  primer 4.3HupEco for PCR amplification
cagagaattcgaggttcagcttcagcagtc

SEQ ID NO: 50 - DNA, 30 bp, artificial sequence
  primer 4.3HDOWNXHO for PCR amplification
gatgctcgagttttggctgaggagacggtg

SEQ ID NO: 51 - DNA, 32 bp, artificial sequence
  primer CH3UPXHOA for PCR amplification
aaaactcgagaaccacaggtgtacaccctgcc

SEQ ID NO: 52 - DNA, 34 bp, artificial sequence
  primer CH3XBA2  for PCR amplification
actgatctagacctttacccggagacagggagag

SEQ ID NO: 53 - DNA, 32 bp, artificial sequence
  primer 4.3LUPECO for PCR amplification
gatagaattcgacattgtgatgacccaggctg

SEQ ID NO: 54 - DNA, 31 bp, artificial sequence
  primer 4.3LDOWNXHO for PCR amplification
attactcgaggagcccgtttcagttccagct

SEQ ID NO: 55 - DNA, 32 bp, artificial sequence
  primer CH3UPXHOB for PCR amplification
gctcctcgagaaccacaggtgtacaccctgcc

SEQ ID NO: 56 - DNA, 43 bp, artificial sequence
  primer CH3STOPKPN  for PCR amplification
acgtggtacctcaggcgcgccctttacccggagacagggagag

SEQ ID NO: 57 - DNA, 32 bp, artificial sequence
  primer EpiTLR1  for PCR amplification
taaagggcgcgcctccggatgccaaatttacc

SEQ ID NO: 58 - DNA, 35 bp, artificial sequence
  primer EpiTLR2 for PCR amplification
tacctcaggcgcgccttattcagtagcagccacac

SEQ ID NO: 59 - protein, 114 aa, artificial sequence
  recombinantly produced antibody
DIVMTQAAPSVPVTPGESVSISCRSSKSLLHTNGNTYLHWFLQRPGQSPQLLIYRMSVLASGVPDRFSGSGSGTAFTLSISRVEAEDVGVFYCMQHLEYPLTFGAGTKLELKRA

SEQ ID NO: 60 - DNA, 342 bp, artificial sequence
  nucleic acid sequence of recombinantly produced antibody
gacattgtgatgacccaggctgcaccctctgtacctgtcactcctggagagtcagtatccatctcctgcaggtctagtaagagtctcctgcatactaatggcaacacttacttgcattggttcctacagaggccaggccagtctcctcagctcctgatatatcggatgtccgtccttgcctcaggagtcccagacaggttcagtggcagtgggtcaggaactgctttcacactgagcatcagtagagtggaggctgaggatgtgggtgttttttactgtatgcaacatctagaatatccgctcacgttcggtgctgggaccaagctggaactgaaacgggct

SEQ ID NO: 61 - protein, 123 aa, artificial sequence
  recombinantly produce antibody
EVQLQQSGPELKKPGETVKISCKASGYTFTNYGMNWVKQAPGKGLKWMGWLNTYTGESIYPDDFKGRFAFSSETSASTAYLQINNLKNEDMATYFCARGDYGYDDPLDYWGQGTSVTVSSAKT

SEQ ID NO: 62 - DNA, 369 bp, artificial sequence
  nucleic acid sequence of recombinantly produced antibody
gaggttcagcttcagcagtctggacctgagctgaagaagcctggagagacagtcaagatctcctgcaaggcttctgggtataccttcacaaactatggaatgaactgggtgaagcaggctccaggaaagggtttaaagtggatgggctggttaaacacctacactggagagtcaatatatcctgatgacttcaagggacggtttgccttctcttcggaaacctctgccagcactgcctatttgcagatcaacaacctcaaaaatgaggacatggctacatatttctgtgcaagaggggactatggttacgacgaccctttggactactggggtcaaggaacctcagtcaccgtctcctcagccaaaaca

SEQ ID NO: 63 - DNA, 6537 bp, artificial sequence
  final expression vector containing TLR9 and CD32 binding regions
agatctaacatccaaagacgaaaggttgaatgaaacctttttgccatccgacatccacaggtccattctcacacataagtgccaaacgcaacaggaggggatacactagcagcagaccgttgcaaacgcaggacctccactcctcttctcctcaacacccacttttgccatcgaaaaaccagcccagttattgggcttgattggagctcgctcattccaattccttctattaggctactaacaccatgactttattagcctgtctatcctggcccccctggcgaggttcatgtttgtttatttccgaatgcaacaagctccgcattacacccgaacatcactccagatgagggctttctgagtgtggggtcaaatagtttcatgttccccaaatggcccaaaactgacagtttaaacgctgtcttggaacctaatatgacaaaagcgtgatctcatccaagatgaactaagtttggttcgttgaaatgctaacggccagttggtcaaaaagaaacttccaaaagtcggcataccgtttgtcttgtttggtattgattgacgaatgctcaaaaataatctcattaatgcttagcgcagtctctctatcgcttctgaaccccggtgcacctgtgccgaaacgcaaatggggaaacacccgctttttggatgattatgcattgtctccacattgtatgcttccaagattctggtgggaatactgctgatagcctaacgttcatgatcaaaatttaactgttctaacccctacttgacagcaatatataaacagaaggaagctgccctgtcttaaacctttttttttatcatcattattagcttactttcataattgcgactggttccaattgacaagcttttgattttaacgacttttaacgacaacttgagaagatcaaaaaacaactaattattcgaaacgatgagatttccttcaatttttactgctgttttattcgcagcatcctccgcattagctgctccagtcaacactacaacagaagatgaaacggcacaaattccggctgaagctgtcatcggttactcagatttagaaggggatttcgatgttgctgttttgccattttccaacagcacaaataacgggttattgtttataaatactactattgccagcattgctgctaaagaagaaggggtatctctcgagaaaagagaggctgaagctgaattcgaggttcagcttcagcagtctggacctgagctgaagaagcctggagagacagtcaagatctcctgcaaggcttctgggtataccttcacaaactatggaatgaactgggtgaagcaggctccaggaaagggtttaaagtggatgggctggttaaacacctacactggagagtcaatatatcctgatgacttcaagggacggtttgccttctcttcggaaacctctgccagcactgcctatttgcagatcaacaacctcaaaaatgaggacatggctacatatttctgtgcaagaggggactatggttacgacgaccctttggactactggggtcaaggaacctcagtcaccgtctcctcagccaaaactcgagaaccacaggtgtacaccctgcccccatcccgggatgagctgggcatcgcgcaagtcagcctgacctgcctggtcaaaggcttctatcccagcgacatcgccgtggagtgggagagcaacgggcagccggagaacaactacaagaccacgcctcccgtgctggactccgacggctctttcttcctctacagcaagcttaccgtgttgggccgcaggtggaccctggggaacgtcttctcatgctccgtgatgcatgaggctctgcacaaccactacacgcagaagagcctctccctgtctccgggtaaatctctagaacaaaaactcatctcagaagaggatctgaatagcgccgtcgaccatcatcatcatcatcattgagtttgtagccttagacatgactgttcctcagttcaagttgggcacttacgagaagaccggtcttgctagattctaatcaagaggatgtcagaatgccatttgcctgagagatgcaggcttcatttttgatacttttttatttgtaacctatatagtataggattttttttgtcattttgtttcttctcgtacgagcttgctcctgatcagcctatctcgcagctgatgaatatcttgtggtaggggtttgggaaaatcattcgagtttgatgtttttcttggtatttcccactcctcttcagagtacagaagattaagtgagaccttcgtttgtgcagatccaacatccaaagacgaaaggttgaatgaaacctttttgccatccgacatccacaggtccattctcacacataagtgccaaacgcaacaggaggggatacactagcagcagaccgttgcaaacgcaggacctccactcctcttctcctcaacacccacttttgccatcgaaaaaccagcccagttattgggcttgattggagctcgctcattccaattccttctattaggctactaacaccatgactttattagcctgtctatcctggcccccctggcgaggttcatgtttgtttatttccgaatgcaacaagctccgcattacacccgaacatcactccagatgagggctttctgagtgtggggtcaaatagtttcatgttccccaaatggcccaaaactgacagtttaaacgctgtcttggaacctaatatgacaaaagcgtgatctcatccaagatgaactaagtttggttcgttgaaatgctaacggccagttggtcaaaaagaaacttccaaaagtcggcataccgtttgtcttgtttggtattgattgacgaatgctcaaaaataatctcattaatgcttagcgcagtctctctatcgcttctgaaccccggtgcacctgtgccgaaacgcaaatggggaaacacccgctttttggatgattatgcattgtctccacattgtatgcttccaagattctggtgggaatactgctgatagcctaacgttcatgatcaaaatttaactgttctaacccctacttgacagcaatatataaacagaaggaagctgccctgtcttaaacctttttttttatcatcattattagcttactttcataattgcgactggttccaattgacaagcttttgattttaacgacttttaacgacaacttgagaagatcaaaaaacaactaattattcgaaacgatgagatttccttcaatttttactgctgttttattcgcagcatcctccgcattagctgctccagtcaacactacaacagaagatgaaacggcacaaattccggctgaagctgtcatcggttactcagatttagaaggggatttcgatgttgctgttttgccattttccaacagcacaaataacgggttattgtttataaatactactattgccagcattgctgctaaagaagaaggggtatctctcgagaaaagagaggctgaagctgaattcgacattgtgatgacccaggctgcaccctctgtacctgtcactcctggagagtcagtatccatctcctgcaggtctagtaagagtctcctgcatactaatggcaacacttacttgcattggttcctacagaggccaggccagtctcctcagctcctgatatatcggatgtccgtccttgcctcaggagtcccagacaggttcagtggcagtgggtcaggaactgctttcacactgagcatcagtagagtggaggctgaggatgtgggtgttttttactgtatgcaacatctagaatatccgctcacgttcggtgctgggaccaagctggaactgaaacgggctcctcgagaaccacaggtgtacaccctgcccccatcccgggatgagctgggcatcgcgcaagtcagcctgacctgcctggtcaaaggcttctatcccagcgacatcgccgtggagtgggagagcaacgggcagccggagaacaactacaagaccacgcctcccgtgctggactccgacggctctttcttcctctacagcaagcttaccgtgttgggccgcaggtggaccctggggaacgtcttctcatgctccgtgatgcatgaggctctgcacaaccactacacgcagaagagcctctccctgtctccgggtaaagggcgcgcctgaggtacctcgagccgcggcggccgccagctttctagaacaaaaactcatctcagaagaggatctgaatagcgccgtcgaccatcatcatcatcatcattgagtttgtagccttagacatgactgttcctcagttcaagttgggcacttacgagaagaccggtcttgctagattctaatcaagaggatgtcagaatgccatttgcctgagagatgcaggcttcatttttgatacttttttatttgtaacctatatagtataggattttttttgtcattttgtttcttctcgtacgagcttgctcctgatcagcctatctcgcagctgatgaatatcttgtggtaggggtttgggaaaatcattcgagtttgatgtttttcttggtatttcccactcctcttcagagtacagaagattaagtgagaccttcgtttgtgcggatcccccacacaccatagcttcaaaatgtttctactccttttttactcttccagattttctcggactccgcgcatcgccgtaccacttcaaaacacccaagcacagcatactaaattttccctctttcttcctctagggtgtcgttaattacccgtactaaaggtttggaaaagaaaaaagagaccgcctcgtttctttttcttcgtcgaaaaaggcaataaaaatttttatcacgtttctttttcttgaaatttttttttttagtttttttctctttcagtgacctccattgatatttaagttaataaacggtcttcaatttctcaagtttcagtttcatttttcttgttctattacaactttttttacttcttgttcattagaaagaaagcatagcaatctaatctaaggggcggtgttgacaattaatcatcggcatagtatatcggcatagtataatacgacaaggtgaggaactaaaccatggccaagttgaccagtgccgttccggtgctcaccgcgcgcgacgtcgccggagcggtcgagttctggaccgaccggctcgggttctcccgggacttcgtggaggacgacttcgccggtgtggtccgggacgacgtgaccctgttcatcagcgcggtccaggaccaggtggtgccggacaacaccctggcctgggtgtgggtgcgcggcctggacgagctgtacgccgagtggtcggaggtcgtgtccacgaacttccgggacgcctccgggccggccatgaccgagatcggcgagcagccgtgggggcgggagttcgccctgcgcgacccggccggcaactgcgtgcacttcgtggccgaggagcaggactgacacgtccgacggcggcccacgggtcccaggcctcggagatccgtcccccttttcctttgtcgatatcatgtaattagttatgtcacgcttacattcacgccctccccccacatccgctctaaccgaaaaggaaggagttagacaacctgaagtctaggtccctatttatttttttatagttatgttagtattaagaacgttatttatatttcaaatttttcttttttttctgtacagacgcgtgtacgcatgtaacattatactgaaaaccttgcttgagaaggttttgggacgctcgaaggctttaatttgcaagctggagaccaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagatc

SEQ ID NO: 64 - DNA, 7046 bp, artificial sequence
  final expression vector containg TLR9 and CD32 binding regions and epitope sequence
agatctaacatccaaagacgaaaggttgaatgaaacctttttgccatccgacatccacaggtccattctcacacataagtgccaaacgcaacaggaggggatacactagcagcagaccgttgcaaacgcaggacctccactcctcttctcctcaacacccacttttgccatcgaaaaaccagcccagttattgggcttgattggagctcgctcattccaattccttctattaggctactaacaccatgactttattagcctgtctatcctggcccccctggcgaggttcatgtttgtttatttccgaatgcaacaagctccgcattacacccgaacatcactccagatgagggctttctgagtgtggggtcaaatagtttcatgttccccaaatggcccaaaactgacagtttaaacgctgtcttggaacctaatatgacaaaagcgtgatctcatccaagatgaactaagtttggttcgttgaaatgctaacggccagttggtcaaaaagaaacttccaaaagtcggcataccgtttgtcttgtttggtattgattgacgaatgctcaaaaataatctcattaatgcttagcgcagtctctctatcgcttctgaaccccggtgcacctgtgccgaaacgcaaatggggaaacacccgctttttggatgattatgcattgtctccacattgtatgcttccaagattctggtgggaatactgctgatagcctaacgttcatgatcaaaatttaactgttctaacccctacttgacagcaatatataaacagaaggaagctgccctgtcttaaacctttttttttatcatcattattagcttactttcataattgcgactggttccaattgacaagcttttgattttaacgacttttaacgacaacttgagaagatcaaaaaacaactaattattcgaaacgatgagatttccttcaatttttactgctgttttattcgcagcatcctccgcattagctgctccagtcaacactacaacagaagatgaaacggcacaaattccggctgaagctgtcatcggttactcagatttagaaggggatttcgatgttgctgttttgccattttccaacagcacaaataacgggttattgtttataaatactactattgccagcattgctgctaaagaagaaggggtatctctcgagaaaagagaggctgaagctgaattcgaggttcagcttcagcagtctggacctgagctgaagaagcctggagagacagtcaagatctcctgcaaggcttctgggtataccttcacaaactatggaatgaactgggtgaagcaggctccaggaaagggtttaaagtggatgggctggttaaacacctacactggagagtcaatatatcctgatgacttcaagggacggtttgccttctcttcggaaacctctgccagcactgcctatttgcagatcaacaacctcaaaaatgaggacatggctacatatttctgtgcaagaggggactatggttacgacgaccctttggactactggggtcaaggaacctcagtcaccgtctcctcagccaaaactcgagaaccacaggtgtacaccctgcccccatcccgggatgagctgggcatcgcgcaagtcagcctgacctgcctggtcaaaggcttctatcccagcgacatcgccgtggagtgggagagcaacgggcagccggagaacaactacaagaccacgcctcccgtgctggactccgacggctctttcttcctctacagcaagcttaccgtgttgggccgcaggtggaccctggggaacgtcttctcatgctccgtgatgcatgaggctctgcacaaccactacacgcagaagagcctctccctgtctccgggtaaatctctagaacaaaaactcatctcagaagaggatctgaatagcgccgtcgaccatcatcatcatcatcattgagtttgtagccttagacatgactgttcctcagttcaagttgggcacttacgagaagaccggtcttgctagattctaatcaagaggatgtcagaatgccatttgcctgagagatgcaggcttcatttttgatacttttttatttgtaacctatatagtataggattttttttgtcattttgtttcttctcgtacgagcttgctcctgatcagcctatctcgcagctgatgaatatcttgtggtaggggtttgggaaaatcattcgagtttgatgtttttcttggtatttcccactcctcttcagagtacagaagattaagtgagaccttcgtttgtgcagatccaacatccaaagacgaaaggttgaatgaaacctttttgccatccgacatccacaggtccattctcacacataagtgccaaacgcaacaggaggggatacactagcagcagaccgttgcaaacgcaggacctccactcctcttctcctcaacacccacttttgccatcgaaaaaccagcccagttattgggcttgattggagctcgctcattccaattccttctattaggctactaacaccatgactttattagcctgtctatcctggcccccctggcgaggttcatgtttgtttatttccgaatgcaacaagctccgcattacacccgaacatcactccagatgagggctttctgagtgtggggtcaaatagtttcatgttccccaaatggcccaaaactgacagtttaaacgctgtcttggaacctaatatgacaaaagcgtgatctcatccaagatgaactaagtttggttcgttgaaatgctaacggccagttggtcaaaaagaaacttccaaaagtcggcataccgtttgtcttgtttggtattgattgacgaatgctcaaaaataatctcattaatgcttagcgcagtctctctatcgcttctgaaccccggtgcacctgtgccgaaacgcaaatggggaaacacccgctttttggatgattatgcattgtctccacattgtatgcttccaagattctggtgggaatactgctgatagcctaacgttcatgatcaaaatttaactgttctaacccctacttgacagcaatatataaacagaaggaagctgccctgtcttaaacctttttttttatcatcattattagcttactttcataattgcgactggttccaattgacaagcttttgattttaacgacttttaacgacaacttgagaagatcaaaaaacaactaattattcgaaacgatgagatttccttcaatttttactgctgttttattcgcagcatcctccgcattagctgctccagtcaacactacaacagaagatgaaacggcacaaattccggctgaagctgtcatcggttactcagatttagaaggggatttcgatgttgctgttttgccattttccaacagcacaaataacgggttattgtttataaatactactattgccagcattgctgctaaagaagaaggggtatctctcgagaaaagagaggctgaagctgaattcgacattgtgatgacccaggctgcaccctctgtacctgtcactcctggagagtcagtatccatctcctgcaggtctagtaagagtctcctgcatactaatggcaacacttacttgcattggttcctacagaggccaggccagtctcctcagctcctgatatatcggatgtccgtccttgcctcaggagtcccagacaggttcagtggcagtgggtcaggaactgctttcacactgagcatcagtagagtggaggctgaggatgtgggtgttttttactgtatgcaacatctagaatatccgctcacgttcggtgctgggaccaagctggaactgaaacgggctcctcgagaaccacaggtgtacaccctgcccccatcccgggatgagctgggcatcgcgcaagtcagcctgacctgcctggtcaaaggcttctatcccagcgacatcgccgtggagtgggagagcaacgggcagccggagaacaactacaagaccacgcctcccgtgctggactccgacggctctttcttcctctacagcaagcttaccgtgttgggccgcaggtggaccctggggaacgtcttctcatgctccgtgatgcatgaggctctgcacaaccactacacgcagaagagcctctccctgtctccgggtaaagggcgcgcctccggatgccaaatttacccgccaaacgcgaacaagatcagagaggctttgcaatcttgcaggaggcccaatgcgcagagattcggcatatccaactactgccagatctaccccccatacgatgggcgtacaatcatacagcgtgataacggctatcagcctaactaccacgccgtgaacatcgtcggctacgagaatgtcgtggttactgtgaaggtaatgggcgatgacggggttctagcttgcgccatagctaccaagtacacttggaacgtacccaaaattgcgccgaaaagtgaaaacgtcgtagtgaccataagggaggcattggctcaacctcaaagatactgcagacactactggacgccctgcataatccaccgtggtaaaccctttcaacttgaggcagtgttcgaagctaacaggacggtaacgccaattcgtatgcaaggtgggtgcgggtcttgttgggctttttctggtgtggctgctactgaataaggcgcgcctgaggtacctcgagccgcggcggccgccagctttctagaacaaaaactcatctcagaagaggatctgaatagcgccgtcgaccatcatcatcatcatcattgagtttgtagccttagacatgactgttcctcagttcaagttgggcacttacgagaagaccggtcttgctagattctaatcaagaggatgtcagaatgccatttgcctgagagatgcaggcttcatttttgatacttttttatttgtaacctatatagtataggattttttttgtcattttgtttcttctcgtacgagcttgctcctgatcagcctatctcgcagctgatgaatatcttgtggtaggggtttgggaaaatcattcgagtttgatgtttttcttggtatttcccactcctcttcagagtacagaagattaagtgagaccttcgtttgtgcggatcccccacacaccatagcttcaaaatgtttctactccttttttactcttccagattttctcggactccgcgcatcgccgtaccacttcaaaacacccaagcacagcatactaaattttccctctttcttcctctagggtgtcgttaattacccgtactaaaggtttggaaaagaaaaaagagaccgcctcgtttctttttcttcgtcgaaaaaggcaataaaaatttttatcacgtttctttttcttgaaatttttttttttagtttttttctctttcagtgacctccattgatatttaagttaataaacggtcttcaatttctcaagtttcagtttcatttttcttgttctattacaactttttttacttcttgttcattagaaagaaagcatagcaatctaatctaaggggcggtgttgacaattaatcatcggcatagtatatcggcatagtataatacgacaaggtgaggaactaaaccatggccaagttgaccagtgccgttccggtgctcaccgcgcgcgacgtcgccggagcggtcgagttctggaccgaccggctcgggttctcccgggacttcgtggaggacgacttcgccggtgtggtccgggacgacgtgaccctgttcatcagcgcggtccaggaccaggtggtgccggacaacaccctggcctgggtgtgggtgcgcggcctggacgagctgtacgccgagtggtcggaggtcgtgtccacgaacttccgggacgcctccgggccggccatgaccgagatcggcgagcagccgtgggggcgggagttcgccctgcgcgacccggccggcaactgcgtgcacttcgtggccgaggagcaggactgacacgtccgacggcggcccacgggtcccaggcctcggagatccgtcccccttttcctttgtcgatatcatgtaattagttatgtcacgcttacattcacgccctccccccacatccgctctaaccgaaaaggaaggagttagacaacctgaagtctaggtccctatttatttttttatagttatgttagtattaagaacgttatttatatttcaaatttttcttttttttctgtacagacgcgtgtacgcatgtaacattatactgaaaaccttgcttgagaaggttttgggacgctcgaaggctttaatttgcaagctggagaccaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagatc

SEQ ID NO: 65 - protein, 12 aa, human
KRADAAPTVSIF

SEQ ID NO: 66 - DNA, 36 bp, human
aaacgggctgatgctgcaccaactgtatccatcttc

SEQ ID NO: 67 - protein, 250 aa, artificial
  ScFV
EVQLQQSGPELKKPGETVKISCKASGYTFTNYGMNWVKQAPGKGLKWMGWLNTYTGESIYPDDFKGRFAFSSETSASTAYLQINNLKNEDMATYFCARGDYGYDDPLDYWGQGTSVTVSSGGGGSGGGGSGGGGSDIVMTQAAPSVPVTPGESVSISCRSSKSLLHTNGNTYLHWFLQRPGQSPQLLIYRMSVLASGVPDRFSGSGSGTAFTLSISRVEAEDVGVFYCMQHLEYPLTFGAGTKLELKGSI

SEQ ID NO: 68 - protein, 19 aa, artificial
  Peptide
ADGAWAWVWLTETAVGAAK

SEQ ID NO: 69 - protein, 249 aa, artificial
  ScFV
EVKLEESGGGLVQPGGSMKLSCVASGFTFSYYWMNWVRQSPEKGLEWVAEIRLKSNNYATHYAESVKGRFTISRDDSKNNVYLQMNNLRAEDTGIYYCNRRDEYYAMDYWGQGTSVSVSSGGGGSGGGGSGGGGSDIVLTQSPGSLAVSLGQRATISCRASESVDNFGISFMNWFQQKPGQPPRLLIYGASNQGSGVPARFSGSGSGTDFSLNIHPVEEDDAAMYFCQQSKEVPWTFGGGTKLEIKGSI

SEQ ID NO: 70 - protein, 230 aa, artificial
  Fc fragment
PKSCDKTHTCPPCPPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 71 - DNA, 20 bp, artificial
  CpG
gggggacgatcgtcgggggg

SEQ ID NO: 72 - DNA, 24 bp, artificial
  CpG
tcgtcgttttgtcgttttgtcgtt

SEQ ID NO: 73 - protein, 12 aa, artificial
  Peptide
ESWDKFLSHYLP

SEQ ID NO: 74 - protein, 7 aa, artificial
  Peptide
TDWSWFY

SEQ ID NO: 75 - protein, 7 aa, artificial
  Peptide
YPVYWPW

SEQ ID NO: 76 - protein, 7 aa, artificial
  Peptide
EWWFYWP

SEQ ID NO: 77 - protein, 7 aa, artificial
  Peptide
WFPIEWW

SEQ ID NO: 78 - protein, 7 aa, artificial
  Peptide
DQVDIGY

SEQ ID NO: 79 - protein, 7 aa, artificial
  Peptide
THQVYIS

SEQ ID NO: 80 - protein, 11 aa, artificial
  Peptide
WFPIEWWFYWP

SEQ ID NO: 81 - protein, 12 aa, artificial
  Peptide
DSWQAFLTKFVL

SEQ ID NO: 82 - protein, 12 aa, artificial
  Peptide
HDIQWFWQHWNS

SEQ ID NO: 83 - protein, 12 aa, artificial
  Peptide
WSWWDHTFNYML

SEQ ID NO: 84 - protein, 12 aa, artificial
  Peptide
TTQQTWNVRYPY

SEQ ID NO: 85 - protein, 12 aa, artificial
  Peptide
DHTMPWTRNAKN

SEQ ID NO: 86 - protein, 12 aa, artificial
  Peptide
SWDPYWPFPWFS

SEQ ID NO: 87 - protein, 12 aa, artificial
  Peptide
AIYYVPSPMFTV

SEQ ID NO: 88 - protein, 12 aa, artificial
  Peptide
ETTLLKMWLAQM

SEQ ID NO: 89 - protein, 12 aa, artificial
  Peptide
YPWLDVAVVSLY

SEQ ID NO: 90 - protein, 12 aa, artificial
  Peptide
VPGWHYLATLRA

SEQ ID NO: 91 - protein, 12 aa, artificial
  Peptide
FDPLGSRDIKGS

SEQ ID NO: 92 - DNA, 25 bp, artificial
  CpG
tcgtcgtcgttcgaacgacgttgat

SEQ ID NO: 93 - protein, 297 aa, artificial
  ScFV
EVQLQQSGPELKKPGETVKISCKASGYTFTNYGMNWVKQAPGKGLKWMGWLNTYTGESIYPDDFKGRFAFSSETSASTAYLQINNLKNEDMATYFCARGDYGYDDPLDYWGQGTSVTVSSGGGGSGGGGSGGGGSDIVMTQAAPSVPVTPGESVSISCRSSKSLLHTNGNTYLHWFLQRPGQSPQLLIYRMSVLASGVPDRFSGSGSGTAFTLSISRVEAEDVGVFYCMQHLEYPLTFGAGTKLELKGSISAWSHPQFEKGPEVSALEKEVSALEKEVSALEKEVSALEKEVSALEK

SEQ ID NO: 94 - protein, 296 aa, artificial
  ScFV
EVKLEESGGGLVQPGGSMKLSCVASGFTFSYYWMNWVRQSPEKGLEWVAEIRLKSNNYATHYAESVKGRFTISRDDSKNNVYLQMNNLRAEDTGIYYCNRRDEYYAMDYWGQGTSVSVSSGGGGSGGGGSGGGGSDIVLTQSPGSLAVSLGQRATISCRASESVDNFGISFMNWFQQKPGQPPRLLIYGASNQGSGVPARFSGSGSGTDFSLNIHPVEEDDAAMYFCQQSKEVPWTFGGGTKLEIKGSISAWSHPQFEKGPEVSALEKEVSALEKEVSALEKEVSALEKEVSALEK

SEQ ID NO: 95 - protein, 56 aa, artificial
  Peptide
ADGAWAWVWLTETAVGAAKGPEVSALEKEVSALEKEVSALEKEVSALEKEVSALEK

SEQ ID NO: 96 - protein, 267 aa, artificial
  Fc fragment-coil
PKSCDKTHTCPPCPPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGPEVSALEKEVSALEKEVSALEKEVSALEKEVSALEK

SEQ ID NO: 97 - protein, 253 aa, artificial
  Immunogen-coil
HHHHHHYYRYVAREQSCRRPNAQRFGISNYCQIYPPNVNKIREALAQTHSAIAVDLRQMRTVTPIRMQGGCGSCWAFSGVAATESAYLQQYDIKYTWNVPKIAPKSENVVVTVKVMGDDGVLACAIATHAKIRDDAFRHYDGRTIIQRDNGYQPNYHAVNIVGYSNAQGVDYWIVRNSWDTNWHEIKKVLVPGCHGSEPCIIHRGKPFGGGSGGGSGGKVSALKEKVSALKEKVSALKEKVSALKEKVSALKE

SEQ ID NO: 98 - protein, 415 aa, artificial
  Immunogen-coil
GVLACAIATHAKIREQERLVKLETVKKSLEQEVRTLHVRIEEVEANALAGGDLRQMRTVTPIRMQGGCGSCWEAHEQQIRIMTTKLKEAEARQQYDIKYTWNVPKIAVNIVGYSNAQGVDYWIVRNSWDTNWYHNPHFIGNRSVITHLMEDLKGELDMRNIQVRGLKQMKRVGDANVKSEDDAFRHYDGRTIIQRDNGYQPNYLDEYWILTAAHCVDGQTVSKLIRSKVLGEKISYYRYVAREQSCRRPNAQRFGISNYCVVVTVKVMGDDELHTYFNVNYTMHYYLNNGATRDILDEYWILTAAHCVAGQTASKLSIRYNSLKHSLFKYRPFKVNELNLEGEFGRELQHKFRLMRNSQMEVEEGGGSHHHHHHGGGSGGKVSALKEKVSALKEKVSALKEKVSALKEKVSALKE

SEQ ID NO: 99 - protein, 10 aa, artificial
  Peptide
SAWSHPQFEK

SEQ ID NO: 100 - protein, 35 aa, artificial
  Peptide
EVSALEKEVSALEKEVSALEKEVSALEKEVSALEK

SEQ ID NO: 101 - protein, 7 aa, artificial
  Peptide
EWWFYWP

SEQ ID NO: 102 - protein, 3 aa, artificial
  Peptide
EWW

SEQ ID NO: 103 - protein, 3 aa, artificial
  Peptide
WFY

SEQ ID NO: 104 - protein, 3 aa, artificial
  Peptide
YWP

SEQ ID NO: 105 - protein, 4 aa, artificial
  Peptide
  Peptide (3)..(3): X can be any amino acid
QVXI